(19) United States
(12) Patent Application Publication
(10) Pub. No.: US 2009/0004185 A1
(43) Pub. Date: Jan. 1, 2009

(54) AMINO-SUBSTITUTED QUINAZOLINE DERIVATIVES AS INHIBITORS OF BETA-CATENIN/TCF-4 PATHWAY AND CANCER TREATMENT AGENTS

(75) Inventors: Aranapakam M. VENKATESAN, Rego Park, NY (US); Christoph DEHNHARDT, New York, NY (US); Zecheng CHEN, New City, NY (US); Osvaldo Dos SANTOS, Astoria, NY (US); Efren Delos SANTOS, Nanuet, NY (US); Kevin CURRAN, Congers, NY (US); Semiramis AYRAL-KALOUSTIAN, Tarrytown, NY (US); Lei CHEN, Hartsdale, NY (US)

Correspondence Address: WilmerHale/Wyeth, 60 STATE STREET, BOSTON, MA, 02109, US

(73) Assignee: WYETH, Madison, NJ (US)

(21) Appl. No.: 11/972,237

(22) Filed: Jan. 10, 2008

Related U.S. Application Data
(60) Provisional application No. 60/879,837, filed on Jan. 11, 2007.

(51) Int. Cl. C07D 239/95; A61K 31/498; C07D 401/02; C07D 403/02; C07D 413/02; A61K 31/5377; A61K 31/55; A61K 39/395; A61P 35/00

(52) U.S. Cl. 424/133.1; 544/291; 514/266.4; 514/266.22; 514/252.17; 544/284; 514/210.21; 514/234.5; 544/119; 540/600; 514/217.06

(57) ABSTRACT

The present invention relates to amino-substituted quinazoline derivatives as inhibitors of β-catenin/tcf-4 pathway, which can be useful in the treatment of cancer; to processes for their preparation; to pharmaceutical compositions comprising them; and to methods of using them.

### CROSS REFERENCE TO RELATED APPLICATIONS

This application claims the benefit of priority under 35 U.S.C. §119(e) to U.S. Patent Application Ser. No. 60/879,837 filed on Jan. 11, 2007, which is hereby incorporated by reference in its entirety.

### FIELD OF THE INVENTION

The present invention relates to amino-substituted quinazoline derivatives as inhibitors of β-catenin/tcf-4 pathway, which can be useful in the treatment of cancer; to processes for their preparation; to pharmaceutical compositions comprising them; and to methods of using them.

### BACKGROUND OF THE INVENTION

Colorectal cancer is the second leading cause of cancer deaths in the United States. Most (85%) colorectal cancers have loss or mutation of tumor suppressor gene Adenomatous Polyposis Coli (APC) which initiates a neoplastic process towards carcinoma formation. APC, along with β-catenin, is a central component of Wnt signaling pathway. The name Wnt was coined as a combination of Wg (wingless) and Int. The wingless gene had originally been identified as a segment polarity gene in Drosophila melanogaster that functions during embryogenesis and also during adult limb formation during metamorphosis. The Int genes were originally identified as vertebrate genes near several integration sites of mouse mammary tumor virus (MMTV). The Int-1 gene and the wingless gene were found to be homologous, with a common evolutionary origin evidenced by similar amino acid sequences of their encoded proteins. Mutations of the wingless gene in the fruit fly were found in wingless flies, while tumors caused by MMTV were found to have copies of the virus integrated into the genome forcing overproduction of one of several Wnt genes. Wnts are a major class of secreted morphogenic ligands of profound importance in establishing the pattern of development in the bodies of all multicellular organisms studied.

Wnt signaling pathway is evolutionally conserved in mammalians, Xenopus, Drosophila and C. elegans. It controls many events during embryonic development and regulates proliferation, morphology, motility and cell fate at a cellular level. Within the pathway, APC in complex with Axin is required to regulate the stability of β-catenin. In the absence of secreted Wnt factors, cytoplasmic β-catenin is phosphorylated by GSK3β kinase in the APC complex and is later subject to rapid protein degradation. In the presence of Wnt factors, the Wnt signaling cascade is activated so that the intrinsic kinase activity of the APC complex is inhibited. As a consequence, stable non-phosphorylated β-catenin accumulates in the cytoplasm and subsequently translocates into nucleus, where it binds to Tcf-4 (T-cell transcriptional factor-4) protein and activates the transcription of a variety of Wnt target genes. Numerous candidate genes have been proposed as critical downstream effectors of Wnt signaling in cancer, including c-myc, cyclin D1, BMP4, KLF4, DHRS9/DHRL, MDR-1, Axin2, GPR49, ROR1, TIMP2, ID2, MSX1, and CSF2.

Therefore, loss of functional APC leads to inappropriate stabilization of β-catenin. In addition, β-catenin can be activated by intragenic mutations that abolish inhibitory phosphorylation sites so that β-catenin is no longer degraded. Activating mutations in β-catenin can occasionally replace inactivating mutations of APC in the initiation of sporadic colorectal cancer (2 to 5% of all colon tumors). Both mutations result in accumulation of non-phosphorylated β-catenin thereby constitutively activating gene transcription and probably promoting carcinogenesis. Introduction of wild type APC into cells which have lost APC function has been shown to result in either growth suppression or apoptosis, suggesting that these cells have become dependent on elevated β-catenin/Tcf-4 signaling.

Accordingly, inhibitors of β-catenin/Tcf-4 pathway can be useful for the treatment of cancer, especially, for the treatment of colorectal cancer.

### SUMMARY OF THE INVENTION

In one aspect, the present invention provides a compound of formula I,

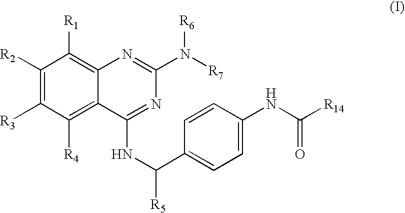

or an enantiomer, diastereomer, tautomer, or pharmaceutically acceptable salt or solvate thereof, wherein the symbols have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
   wherein: R₂ and R₃ together with the two contiguous carbon atoms to which R₂ and R₃ are bonded may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
each occurrence of Rₐ is independently hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
each occurrence of R_{b}, R_{c} and R_{d} is independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
each occurrence of Rₑ is independently alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In certain embodiments, R₁, R₂, R₃, and R₄ of the compound of formula I are each independently hydrogen, halogen, cyano, nitro, CF₃, OCF₃, C₁-C₄ alkyl or substituted C₁-C₄ alkyl, C₂-C₆ alkenyl or substituted C₂-C₆ alkenyl, C₂-C₆ alkynyl or substituted C₂-C₆ alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, S(═O)₂ORₑ, NR_{b}R_{c}, NR_{b}S(═O)₂Rₑ, S(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, or NR_{b}C(═O)Rₐ.

In certain other embodiments, R₂ and R₃ of the compound of formula I are each independently hydrogen, halogen, cyano, nitro, CF₃, OCF₃, C₁-C₄ alkyl or substituted C₁-C₄ alkyl, C₂-C₆ alkenyl or substituted C₂-C₆ alkenyl, C₂-C₆ alkynyl or substituted C₂-C₆ alkynyl, C₃-C₇ cycloalkyl or substituted C₃-C₇ cycloalkyl, heterocyclyl or substituted heterocyclyl, aryl or substituted aryl, ORₐ, C(═O)ORₑ, or C(═O)Rₐ.

In certain embodiments, R₆ and R₇ of the compound of formula I are each independently hydrogen, C₁-C₄ alkyl or substituted C₁-C₄ alkyl, C₃-C₇ cycloalkyl or substituted C₃-C₇ cycloalkyl, or said R₆ and R₇ together with the N to which they are bonded optionally form a heterocycle or substituted heterocycle, in which said heterocycle is fully saturated or partially unsaturated.

In certain other embodiments, R₅ of the compound of formula I is hydrogen or methyl. In some embodiments, R₁₄ of the compound of formula I is heteroaryl or substituted heteroaryl. In some other embodiments, R₁₄ of the compound of formula I is heterocycle or substituted heterocycle, in which said heterocycle is fully saturated. In certain embodiments, R₁₄ of the compound of formula I is phenyl or substituted phenyl. In certain other embodiments, R₁₄ of the compound of formula I is pyridinyl or substituted pyridinyl.

In certain embodiments, R₁₄ of the compound of formula I is piperidinyl or substituted piperidinyl. In certain other embodiments, R₁₄ of the compound of formula I is:

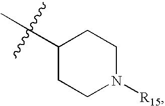

wherein R₁₅ is hydrogen, C₁-C₄ alkyl, C₃-C₇ cycloalkyl, —CH₂-phenyl or —CH₂-substituted phenyl, or —CH₂-heteroaryl or —CH₂-substituted heteroaryl. In some embodiments, R₁₄ of the compound of formula I is —NH-aryl or —NH-substituted aryl. In some other embodiments, R₁₄ of the compound of formula I is —NH-phenyl or —NH-substituted phenyl.

In another aspect, the present invention provides a compound of formula I, or an enantiomer, diastereomer, tautomer, or pharmaceutically acceptable salt or solvate thereof, wherein the compound of formula I is selected from the group consisting of:
5-fluoro-2-methyl-N-[4-({[2-(methylamino)quinazolin-4-yl]amino}methyl)phenyl]benzamide;
2-(benzyloxy)-N-[4-({[2-(methylamino)quinazolin-4-yl]amino}methyl)phenyl]acetamide;
6-chloro-N-[4-({[2-(methylamino)quinazolin-4-yl]amino}methyl)phenyl]nicotinamide;
2-chloro-N-[4-({[2-(methylamino)quinazolin-4-yl]amino}methyl)phenyl]isonicotinamide;
N-[4-({[2-(methylamino)quinazolin-4-yl]amino}methyl)phenyl]quinoline-2-carboxamide;
2-chloro-5-fluoro-N-[4-({[2-(methylamino)quinazolin-4-yl]amino}methyl)phenyl]benzamide;
2-chloro-N-[4-({[2-(methylamino)quinazolin-4-yl]amino}methyl)phenyl]nicotinamide;
2,6-dichloro-5-fluoro-N-[4-({[2-(methylamino)quinazolin-4-yl]amino}methyl)phenyl]nicotinamide;
6-chloro-N-[4-({[6,7-dimethoxy-2-(methylamino)quinazolin-4-yl]amino}methyl)phenyl]nicotinamide;
4-chloro-N-[4-({[2-(methylamino)quinazolin-4-yl]amino}methyl)phenyl]benzamide;
3-chloro-N-[4-({[2-(methylamino)quinazolin-4-yl]amino}methyl)phenyl]benzamide;
4-methyl-N-[4-({[2-(methylamino)quinazolin-4-yl]amino}methyl)phenyl]benzamide;
4-fluoro-N-[4-({[2-(methylamino)quinazolin-4-yl]amino}methyl)phenyl]benzamide;
4-chloro-2-fluoro-N-[4-({[2-(methylamino)quinazolin-4-yl]amino}methyl)phenyl]benzamide;
N-[4-({[2-(methylamino)quinazolin-4-yl]amino}methyl)phenyl]biphenyl-4-carboxamide;
2,4-dichloro-N-[4-({[2-(methylamino)quinazolin-4-yl]amino}methyl)phenyl]benzamide;
4-fluoro-3-methyl-N-[4-({[2-(methylamino)quinazolin-4-yl]amino}methyl)phenyl]benzamide;
4-chloro-N-[4-({[6-methyl-2-(methylamino)quinazolin-4-yl]amino}methyl)phenyl]benzamide;
2,4-dichloro-N-[4-({[6-methyl-2-(methylamino)quinazolin-4-yl]amino}methyl)phenyl]benzamide;
N-[4-({[2-(methylamino)quinazolin-4-yl]amino}methyl)phenyl]-4-(trifluoromethyl)benzamide;
4-cyano-N-[4-({[2-(methylamino)quinazolin-4-yl]amino}methyl)phenyl]benzamide;
N-[4-({[2-(methylamino)quinazolin-4-yl]amino}methyl)phenyl]-4-(trifluoromethoxy)benzamide;
6-chloro-N-[4-({[6,8-dimethyl-2-(methylamino)quinazolin-4-yl]amino}methyl)phenyl]nicotinamide;
4-fluoro-N-[4-({[6-methyl-2-(methylamino)quinazolin-4-yl]amino}methyl)phenyl]benzamide;
4-cyano-N-[4-({[6-methyl-2-(methylamino)quinazolin-4-yl]amino}methyl)phenyl]benzamide;
4-chloro-2-fluoro-N-[4-({[6-methyl-2-(methylamino)quinazolin-4-yl]amino}methyl)phenyl]benzamide;
N-[4-({[6-methyl-2-(methylamino)quinazolin-4-yl]amino}methyl)phenyl]-4-(trifluoromethyl)benzamide;
2-chloro-4-fluoro-N-[4-({[6-methyl-2-(methylamino)quinazolin-4-yl]amino}methyl)phenyl]benzamide;
4-chloro-N-[4-({[6,8-dimethyl-2-(methylamino)quinazolin-4-yl]amino}methyl)phenyl]benzamide;
N-[4-({[6,8-dimethyl-2-(methylamino)quinazolin-4-yl]amino}methyl)phenyl]-4-fluorobenzamide;
6-chloro-N-[4-({[6-methoxy-2-(methylamino)quinazolin-4-yl]amino}methyl)phenyl]nicotinamide;
3,4-difluoro-N-[4-({[6-methyl-2-(methylamino)quinazolin-4-yl]amino}methyl)phenyl]benzamide;
4-chloro-N-[4-({[6-methoxy-2-(methylamino)quinazolin-4-yl]amino}methyl)phenyl]benzamide;
2,4-difluoro-N-[4-({[6-methyl-2-(methylamino)quinazolin-4-yl]amino}methyl)phenyl]benzamide;
4-chloro-N-[4-({[6,8-dimethyl-2-(methylamino)quinazolin-4-yl]amino}methyl)phenyl]-2-fluorobenzamide;
N-[4-({[2-(dimethylamino)-6-methylquinazolin-4-yl]amino}methyl)phenyl]-3,4-difluorobenzamide;
3,4-dichloro-N-[4-({[6-methyl-2-(methylamino)quinazolin-4-yl]amino}methyl)phenyl]benzamide;
N-[4-({[2-(dimethylamino)-6-methylquinazolin-4-yl]amino}methyl)phenyl]-4-fluorobenzamide;
3,5-difluoro-N-[4-({[6-methyl-2-(methylamino)quinazolin-4-yl]amino}methyl)phenyl]benzamide;
4-fluoro-N-(4-{[(6-methyl-2-piperidin-1-ylquinazolin-4-yl)amino]methyl}phenyl)benzamide;
N-[4-((2-(diethylamino)-6-methylquinazolin-4-ylamino)methyl)phenyl)-4-fluorobenzamide;
N-(4-((2-(1-azacyclopentyl)-6-methylquinazolin-4-ylamino)methyl)phenyl)-4-fluorobenzamide;
4-fluoro-N-(4((6-methyl-2-piperazin-1-yl)quinazolin-4-ylamino)methyl)phenyl)benzamide;
4-fluoro-N-((6-methyl-2-(4-methylpiperazin-1-yl)quinazolin-4-ylamino)methyl-phenyl)benzamide;
2-fluoro-N-[4-({[6-methyl-2-(methylamino)quinazolin-4-yl]amino}methyl)phenyl]benzamide;
4-fluoro-N-[4-({[6-methyl-2-(4-methylpiperazin-1-yl)quinazolin-4-yl]amino}methyl)phenyl]benzamide;
N-[4-({[6,8-dimethyl-2-(4-methylpiperazin-1-yl)quinazolin-4-yl]amino}methyl)phenyl]-4-fluorobenzamide;
N-{4-[({6,8-dimethyl-2-[(3S)-3-methylpiperazin-1-yl]quinazolin-4-yl}amino)methyl]phenyl}-4-fluorobenzamide;
4-fluoro-N-{4-[({6-methyl-2-[(3S)-3-methylpiperazin-1-yl]quinazolin-4-yl}amino)methyl]phenyl}benzamide;
N-[4-({[2-(dimethylamino)-6,8-dimethylquinazolin-4-yl]amino}methyl)phenyl]-4-fluorobenzamide;
4-chloro-N-[4-({[2-(dimethylamino)-6-methylquinazolin-4-yl]amino}methyl)phenyl]benzamide;
Ethyl 4-[4-({4-[(4-fluorobenzoyl)amino]benzyl}amino)-6-methylquinazolin-2-yl]piperazine-1-carboxylate;
4-fluoro-N-[4-({[6-methyl-2-(4-pyridin-2-ylpiperazin-1-yl)quinazolin-4-yl]amino}methyl)phenyl]benzamide;
N-(4-{[(2-azepan-1-yl-6-methylquinazolin-4-yl)amino]methyl}phenyl)-4-fluorobenzamide;
N-[4-({[2-(4-ethylpiperazin-1-yl)-6-methylquinazolin-4-yl]amino}methyl)phenyl]-4-fluorobenzamide; and
4-fluoro-N-{4-[({6-methyl-2-[methyl(pyridin-2-ylmethyl)amino]quinazolin-4-yl}amino)methyl]phenyl}benzamide.

In yet another aspect, the present invention provides a compound of formula I, or an enantiomer, diastereomer, tautomer, or pharmaceutically acceptable salt or solvate thereof, wherein the compound of formula I is selected from the group consisting of:
N-{4-[({2-(dimethylamino)-6-[6-(dimethylamino)pyridin-3-yl]quinazolin-4-yl}amino)methyl]phenyl}-4-fluorobenzamide;
N-[4-({[2-(dimethylamino)-6-fluoroquinazolin-4-yl]amino}methyl)phenyl]-4-fluorobenzamide;
N-[4-({[2-(dimethylamino)-7-isopropylquinazolin-4-yl]amino}methyl)phenyl]-4-fluorobenzamide;
6-chloro-N-[4-({[2-(dimethylamino)-7-isopropylquinazolin-4-yl]amino}methyl)phenyl]nicotinamide;
1-benzyl-N-[4-({[2-(dimethylamino)-7-isopropylquinazolin-4-yl]amino}methyl)phenyl]piperidine-4-carboxamide;
6-chloro-N-[4-({[2-(dimethylamino)-7-fluoro-8-methylquinazolin-4-yl]amino}methyl)phenyl]nicotinamide;
6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide;
N-[4-({[6-bromo-8-methyl-2-(methylamino)quinazolin-4-yl]amino}methyl)phenyl]-4-chlorobenzamide;
4-chloro-N-[4-({[6-(2-furyl)-8-methyl-2-(methylamino)quinazolin-4-yl]amino}methyl)phenyl]benzamide;
N-[4-({[2-(dimethylamino)-6-iodoquinazolin-4-yl]amino}methyl)phenyl]-4-fluorobenzamide;
N-{4-[({2-(dimethylamino)-6-[3-(dimethylamino)prop-1-yn-1-yl]quinazolin-4-yl}amino)methyl]phenyl}-4-fluorobenzamide;
Methyl 2-(dimethylamino)-4-({4-[(4-fluorobenzoyl)amino]benzyl}amino)quinazoline-6-carboxylate;
N-[4-({[2-(dimethylamino)-6-(hydroxymethyl)quinazolin-4-yl]amino}methyl)phenyl]-4-fluorobenzamide;
6-chloro-N-[4-({[2-(dimethylamino)-6-iodoquinazolin-4-yl]amino}methyl)phenyl]nicotinamide;
6-chloro-N-[4-({[2-(dimethylamino)-6-vinylquinazolin-4-yl]amino}methyl)phenyl]nicotinamide;
1-benzyl-N-[4-({[2-(dimethylamino)-6-iodoquinazolin-4-yl]amino}methyl)phenyl]piperidine-4-carboxamide;
1-benzyl-N-[4-({[2-(dimethylamino)-6-vinylquinazolin-4-yl]amino}methyl)phenyl]piperidine-4-carboxamide;
1-benzyl-N-[4-({[2-(dimethylamino)-8-methylquinazolin-4-yl]amino}methyl)phenyl]piperidine-4-carboxamide;
1-benzyl-N-(4-{[(6-methyl-2-pyrrolidin-1-ylquinazolin-4-yl)amino]methyl}phenyl)piperidine-4-carboxamide;
1-benzyl-N-{4-[({2-[(3R,5S)-3,5-dimethylpiperazin-1-yl]-6-methylquinazolin-4-yl}amino)methyl]phenyl}piperidine-4-carboxamide;
1-benzyl-N-[4-({[6-methyl-2-(4-pyridin-2-ylpiperazin-1-yl)quinazolin-4-yl]amino}methyl)phenyl]piperidine-4-carboxamide;
1-benzyl-N-[4-({[2-(2,5-dihydro-1H-pyrrol-1-yl)-6-methylquinazolin-4-yl]amino}methyl)phenyl]piperidine-4-carboxamide;
1-benzyl-N-{4-[({2-[(2-furylmethyl)amino]-6-methylquinazolin-4-yl}amino)methyl]phenyl}piperidine-4-carboxamide;
1-benzyl-N-[4-({[6-methyl-2-(4-pyrimidin-2-ylpiperazin-1-yl)quinazolin-4-yl]amino}methyl)phenyl]piperidine-4-carboxamide;
1-benzyl-N-[4-({[6-methyl-2-(4-pyrrolidin-1-ylpiperidin-1-yl)quinazolin-4-yl]amino}methyl)phenyl]piperidine-4-carboxamide;
N-(4-{[(2-azetidin-1-yl-6-methylquinazolin-4-yl)amino]methyl}phenyl)-1-benzylpiperidine-4-carboxamide;
1-benzyl-N-{4-[({6-methyl-2-[(3R)-3-methylpiperazin-1-yl]quinazolin-4-yl}amino)methyl]phenyl}piperidine-4-carboxamide;
N-[4-({[2-(dimethylamino)-7-iodoquinazolin-4-yl]amino}methyl)phenyl]-4-fluorobenzamide;
N-[4-({[2-(dimethylamino)-7-vinylquinazolin-4-yl]amino}methyl)phenyl]-4-fluorobenzamide;
N-[4-({[2-(dimethylamino)-7-ethylquinazolin-4-yl]amino}methyl)phenyl]-4-fluorobenzamide;
N-[4-({[7-cyano-2-(dimethylamino)quinazolin-4-yl]amino}methyl)phenyl]-4-fluorobenzamide;
N-[4-({[7-(aminomethyl)-2-(dimethylamino)quinazolin-4-yl]amino}methyl)phenyl]-4-fluorobenzamide;
N-{4-[({2-(dimethylamino)-7-[(dimethylamino)methyl]quinazolin-4-yl}amino)methyl]phenyl}-4-fluorobenzamide;
N-[4-({[2-(dimethylamino)-7-formylquinazolin-4-yl]amino}methyl)phenyl]-4-fluorobenzamide;
N-[4-({[2-(dimethylamino)-7-(hydroxymethyl)quinazolin-4-yl]amino}methyl)phenyl]-4-fluorobenzamide;
N-[4-({[7-acetyl-2-(dimethylamino)quinazolin-4-yl]amino}methyl)phenyl]-4-fluorobenzamide;
N-[4-({[2-(dimethylamino)-7-(1-hydroxyethyl)quinazolin-4-yl]amino}methyl)phenyl]-4-fluorobenzamide;
N-{4-[({2-(dimethylamino)-7-[(1E)-prop-1-en-1-yl]quinazolin-4-yl}amino)methyl]phenyl}-4-fluorobenzamide;
N-{4-[({2-(dimethylamino)-7-[(1Z)-prop-1-en-1-yl]quinazolin-4-yl}amino)methyl]phenyl}-4-fluorobenzamide;
N-[4-({[2-(dimethylamino)-7-(2-formylphenyl)quinazolin-4-yl]amino}methyl)phenyl]-4-fluorobenzamide;
N-[4-({[2-(dimethylamino)-7-(4-formylphenyl)quinazolin-4-yl]amino}methyl)phenyl]-4-fluorobenzamide;
N-[4-({[7-(2-chloropyridin-3-yl)-2-(dimethylamino)quinazolin-4-yl]amino}methyl)phenyl]-4-fluorobenzamide;
N-[4-({[7-(1-benzofuran-2-yl)-2-(dimethylamino)quinazolin-4-yl]amino}methyl)phenyl]-4-fluorobenzamide;
N-{4-[({2-(dimethylamino)-7-[(1E)-3,3-dimethylbut-1-en-1-yl]quinazolin-4-yl}amino)methyl]phenyl}-4-fluorobenzamide;
N-{4-[({2-(dimethylamino)-7-[(1E)-hex-1-en-1-yl]quinazolin-4-yl}amino)methyl]phenyl}-4-fluorobenzamide;
N-[4-({[7-cyclopropyl-2-(dimethylamino)quinazolin-4-yl]amino}methyl)phenyl]-4-fluorobenzamide;
6-chloro-N-[4-((1S)-1-{[2-(dimethylamino)-7-iodoquinazolin-4-yl]amino}ethyl)phenyl]nicotinamide;
6-chloro-N-[4-((1S)-1-{[2-(dimethylamino)-7-vinylquinazolin-4-yl]amino}ethyl)phenyl]nicotinamide;
6-chloro-N-{4-[(1S)-1-({2-(dimethylamino)-7-[(1E)-prop-1-en-1-yl]quinazolin-4-yl}amino)ethyl]phenyl}nicotinamide;
N-[4-({[2-(dimethylamino)-7-ethynylquinazolin-4-yl]amino}methyl)phenyl]-4-fluorobenzamide;
6-chloro-N-(4-{[(2-chloro-7-iodoquinazolin-4-yl)amino]methyl}phenyl)nicotinamide;
6-chloro-N-[4-({[2-(dimethylamino)-7-iodoquinazolin-4-yl]amino}methyl)phenyl]nicotinamide;
6-chloro-N-[4-({[2-(dimethylamino)-7-vinylquinazolin-4-yl]amino}methyl)phenyl]nicotinamide;
6-chloro-N-{4-[({2-(dimethylamino)-7-[(1E)-prop-1-en-1-yl]quinazolin-4-yl}amino)methyl]phenyl}nicotinamide;
N-[4-({[2-(dimethylamino)quinazolin-4-yl]amino}methyl)phenyl]-4-fluorobenzamide;
N-(4-{[(2-azetidin-1-ylquinazolin-4-yl)amino]methyl}phenyl)-4-fluorobenzamide;
N-[4-({[2-(cyclobutylamino)quinazolin-4-yl]amino}methyl)phenyl]-4-fluorobenzamide;
4-fluoro-N-[4-({[2-(4-methylpiperazin-1-yl)quinazolin-4-yl]amino}methyl)phenyl]benzamide;
4-fluoro-N-(4-{[(2-morpholin-4-ylquinazolin-4-yl)amino]methyl}phenyl)benzamide; and
N-[4-({[2-(ethylamino)quinazolin-4-yl]amino}methyl)phenyl]-4-fluorobenzamide.

In yet another aspect, the present invention provides a compound of formula I, or an enantiomer, diastereomer, tautomer, or pharmaceutically acceptable salt or solvate thereof, wherein the compound of formula I is selected from the group consisting of:
4-fluoro-N-(4-{[(2-pyrrolidin-1-ylquinazolin-4-yl)amino]methyl}phenyl)benzamide;
N-[4-({[2-(cyclopentylamino)quinazolin-4-yl]amino}methyl)phenyl]-4-fluorobenzamide;
N-[4-({[2-(cyclopropylamino)quinazolin-4-yl]amino}methyl)phenyl]-4-fluorobenzamide;
N-[4-({[2-(diethylamino)quinazolin-4-yl]amino}methyl)phenyl]-4-fluorobenzamide;
4-fluoro-N-(4-{[(2-piperidin-1-ylquinazolin-4-yl)amino]methyl}phenyl)benzamide;
4-fluoro-N-{4-[({2-[(2-furylmethyl)amino]quinazolin-4-yl}amino)methyl]phenyl}benzamide;
N-[4-({[2-(cyclohexylamino)quinazolin-4-yl]amino}methyl)phenyl]-4-fluorobenzamide;
tert-butyl N-[4-({4-[(4-fluorobenzoyl)amino]benzyl}amino)quinazolin-2-yl]glycinate;
N-[4-({4-[(4-fluorobenzoyl)amino]benzyl}amino)quinazolin-2-yl]glycine;
6-chloro-N-[4-({[2-(dimethylamino)quinazolin-4-yl]amino}methyl)phenyl]nicotinamide;
1-benzyl-N-[4-({[2-(dimethylamino)-7-methylquinazolin-4-yl]amino}methyl)phenyl]piperidine-4-carboxamide;
1-benzyl-N-[4-({[7-methyl-2-(methylamino)quinazolin-4-yl]amino}methyl)phenyl]piperidine-4-carboxamide;
N-(4-{[(2-azepan-1-yl-7-methylquinazolin-4-yl)amino]methyl}phenyl)-1-benzylpiperidine-4-carboxamide;
1-benzyl-N-[4-({[2-(ethylamino)-7-methylquinazolin-4-yl]amino}methyl)phenyl]piperidine-4-carboxamide;
1-benzyl-N-(4-{[(7-methyl-2-pyrrolidin-1-ylquinazolin-4-yl)amino]methyl}phenyl)piperidine-4-carboxamide;
N-(4-{[(2-azetidin-1-yl-7-methylquinazolin-4-yl)amino]methyl}phenyl)-1-benzylpiperidine-4-carboxamide;
1-benzyl-N-[4-({[7-methyl-2-(4-pyrrolidin-1-ylpiperidin-1-yl)quinazolin-4-yl]amino}methyl)phenyl]piperidine-4-carboxamide;
1-benzyl-N-[4-({[7-methyl-2-(4-pyrimidin-2-ylpiperazin-1-yl)quinazolin-4-yl]aminol}methyl)phenyl]piperidine-4-carboxamide;
N-(4-{[(2-azetidin-1-yl-7-methylquinazolin-4-yl)amino]methyl}phenyl)-1-benzylpiperidine-4-carboxamide;
1-benzyl-N-{4-[({2-[3-(2-hydroxyethyl)piperazin-1-yl]-7-methylquinazolin-4-yl}amino)methyl]phenyl}piperidine-4-carboxamide;
1-benzyl-N-{4-[({2-[(2-hydroxyethyl)(methyl)amino]-7-methylquinazolin-4-yl}amino)methyl]phenyl}piperidine-4-carboxamide;
1-benzyl-N-{4-[({2-[[3-(dimethylamino)propyl](methyl)amino]-7-methylquinazolin-4-yl}amino)methyl]phenyl}piperidine-4-carboxamide;
1-benzyl-N-[4-({[7-methyl-2-(4-methylpiperazin-1-yl)quinazolin-4-yl]amino}methyl)phenyl]piperidine-4-carboxamide;
1-benzyl-N-{4-[({2-[benzyl(methyl)amino]-7-methylquinazolin-4-yl}amino)methyl]phenyl}piperidine-4-carboxamide;
1-benzyl-N-{4-[({7-methyl-2-[(3R)-3-methylpiperazin-1-yl]quinazolin-4-yl}amino)methyl]phenyl}piperidine-4-carboxamide;
1-benzyl-N-{4-[({2-[[2-(dimethylamino)ethyl](methyl)amino]-7-methylquinazolin-4-yl}amino)methyl]phenyl}piperidine-4-carboxamide;
1-benzyl-N-{4-[({7-methyl-2-[methyl(2-pyridin-2-ylethyl)amino]quinazolin-4-yl}amino)methyl]phenyl}piperidine-4-carboxamide;
1-benzyl-N-[4-({[2-(dimethylamino)quinazolin-4-yl]amino}methyl)phenyl]piperidine-4-carboxamide;
1-benzyl-N-[4-({[2-(methylamino)quinazolin-4-yl]amino}methyl)phenyl]piperidine-4-carboxamide;
1-benzyl-N-[4-({[2-(dimethylamino)-6-methylquinazolin-4-yl]amino}methyl)phenyl]piperidine-4-carboxamide;
1-benzyl-N-[4-({[6-methyl-2-(methylamino)quinazolin-4-yl]amino}methyl)phenyl]piperidine-4-carboxamide;
1-benzyl-N-[4-({[2-(dimethylamino)-7-vinylquinazolin-4-yl]amino}methyl)phenyl]piperidine-4-carboxamide;
1-benzyl-N-{4-[({2-(dimethylamino)-7-[(1E)-prop-1-en-1-yl]quinazolin-4-yl}amino)methyl]phenyl}piperidine-4-carboxamide;
1-benzyl-N-{4-[({2-(dimethylamino)-7-[(1E)-3,3-dimethylbut-1-en-1-yl]quinazolin-4-yl}amino)methyl]phenyl}piperidine-4-carboxamide;
1-benzyl-N-{4-[({2-(dimethylamino)-7-[(1Z)-prop-1-en-1-yl]quinazolin-4-yl}amino)methyl]phenyl}piperidine-4-carboxamide;
N-[4-({[2-(dimethylamino)-6-(trifluoromethyl)quinazolin-4-yl]amino}methyl)phenyl]-1-(4-fluorobenzyl)piperidine-4-carboxamide;
N-[4-({[2-azetidin-1-yl-6-(trifluoromethyl)quinazolin-4-yl]amino}methyl)phenyl]-1-(4-fluorobenzyl)piperidine-4-carboxamide;
1-(4-fluorobenzyl)-N-[4-({[2-pyrrolidin-1-yl-6-(trifluoromethyl)quinazolin-4-yl]amino}methyl)phenyl]piperidine-4-carboxamide;
1-(4-fluorobenzyl)-N-[4-({[2-(4-pyrimidin-2-ylpiperazin-1-yl)-6-(trifluoromethyl)quinazolin-4-yl]amino}methyl)phenyl]piperidine-4-carboxamide;
N-[4-({[2-diethylamino)-6-(trifluoromethyl)quinazolin-4-yl]amino}methyl)phenyl]-1-(4-fluorobenzyl)piperidine-4-carboxamide;
N-[4-({[2-(cyclobutylamino)-6-(trifluoromethyl)quinazolin-4-yl]amino}methyl)phenyl]-1-(4-fluorobenzyl)piperidine-4-carboxamide;
1-(4-fluorobenzyl)-N-[4-({[2-(methylamino)-6-(trifluoromethyl)quinazolin-4-yl]amino}methyl)phenyl]piperidine-4-carboxamide;
4-chloro-N-[4-({[2-(dimethylamino)-6-(trifluoromethyl)quinazolin-4-yl]amino}methyl)phenyl]benzamide;
N-[4-({[2-azetidin-1-yl-6-(trifluoromethyl)quinazolin-4-yl]amino}methyl)phenyl]-4-chlorobenzamide;
4-chloro-N-[4-({[2-pyrrolidin-1-yl-6-(trifluoromethyl)quinazolin-4-yl]amino}methyl)phenyl]benzamide;
4-chloro-N-[4-({[2-(4-pyrimidin-2-ylpiperazin-1-yl)-6-(trifluoromethyl)quinazolin-4-yl]amino}methyl)phenyl]benzamide;
4-chloro-N-[4-({[2-(diethylamino)-6-(trifluoromethyl)quinazolin-4-yl]amino}methyl)phenyl]benzamide;
4-chloro-N-[4-({[2-(cyclobutylamino)-6-(trifluoromethyl)quinazolin-4-yl]amino}methyl)phenyl]benzamide;
4-chloro-N-[4-({[2-(methylamino)-6-(trifluoromethyl)quinazolin-4-yl]amino}methyl)phenyl]benzamide;
4-chloro-N-[4-({[2-[(2-furylmethyl)amino]-6-(trifluoromethyl)quinazolin-4-yl]amino}methyl)phenyl]benzamide;
N-(4-((2-dimethylamino)-6-(5-(dimethylamino)pyridine-2-yl)quinazolin-4-ylamino)methyl)phenyl)-4-fluorobenzamide;
4-((2-dimethylamino)-6-(3-(dimethylamino)phenyl)quinazolin-4-ylamino)methyl)N-4-fluorophenyl)benzamide;
Z-(4-((2-dimethylamino)-6-(styrylquinazolin-4-ylamino)-N-(4-fluorophenyl)benzamide;
4-((2-dimethylamino)-6-(3-vinylphenyl)quinazolin-4-ylamino)methyl)-N-(4-fluorophenyl)benzamide; and
E-(4-((2-dimethylamino)-6-(4-styrylphenyl)quinazolin-4-ylamino)methyl-N-(4-fluorophenyl)benzamide.

In yet another aspect, the present invention provides a compound of formula I, or an enantiomer, diastereomer, tautomer, or pharmaceutically acceptable salt or solvate thereof, wherein the compound of formula I is selected from the group consisting of:
E-4-((2-dimethylamino)-6-(prop-1-enyl)quinazolin-4-ylamino)methyl-N-(4-fluorophenyl)benzamide;
E-(4-((2-dimethylamino)-6-(hex-1-enyl)quinazolin-4-ylamino)methyl)-N-(4-fluorophenyl)benzamide;
(E)-N-{4-[({2-(dimethylamino)-6-(3,3-dimethylbut-1-enyl)quinazolin-4-ylamino)methyl)-N-(4-fluorophenyl)benzamide;
N-(4-chlorophenyl)-N′-[4-({[2-(dimethylamino)-7-methylquinazolin-4-yl]amino}methyl)phenyl]urea;
N-(4-chlorophenyl)-N′-[4-({[2-(dimethylamino)-6-methylquinazolin-4-yl]amino}methyl)phenyl]urea);
(N-(4-bromophenyl)-N′-[4-({[2-(dimethylamino)-7-methylquinazolin-4-yl]amino}methyl)phenyl]urea);
N-[4-({[2-(dimethylamino)-7-methylquinazolin-4-yl]amino}methyl)phenyl]-N′-(4-fluorophenyl)urea);
N-[4-({[2-(dimethylamino)-6-methylquinazolin-4-yl]amino}methyl)phenyl]-N′-(4-fluorophenyl)urea);
N-[4-({[2-(dimethylamino)quinazolin-4-yl]amino}methyl)phenyl]-N′-(4-fluorophenyl)urea;
6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide;
6-chloro-N-[4-({[2-(methylamino)-6-nitroquinazolin-4-yl]amino}methyl)phenyl]nicotinamide;
6-chloro-N-[4-({[2-(methylamino)-8-nitroquinazolin-4-yl]amino}methyl)phenyl]nicotinamide;
4-fluoro-N-[4-({[2-(methylamino)-6-nitroquinazolin-4-yl]amino}methyl)phenyl]benzamide;
N-[4-({[2-(dimethylamino)-6-nitroquinazolin-4-yl]amino}methyl)phenyl]-4-fluorobenzamide;
N-[4-({[6-nitro-2-(methylamino)quinazolin-4-yl]amino}methyl)phenyl]nicotinamide;
3,4-difluoro-N-[4-({[5-methyl-2-(methylamino)quinazolin-4-yl]amino}methyl)phenyl]benzamide;
4-fluoro-N-[4-({[8-methyl-2-(methylamino)quinazolin-4-yl]amino}methyl)phenyl]benzamide;
4-chloro-N-[4-({[8-methyl-2-(methylamino)quinazolin-4-yl]amino}methyl)phenyl]benzamide;
4-fluoro-N-[4-({[5-methyl-2-(methylamino)quinazolin-4-yl]amino}methyl)phenyl]benzamide;
4-chloro-N-[4-({[5-methyl-2-(methylamino)quinazolin-4-yl]amino}methyl)phenyl]benzamide;
6-chloro-N-[4-({[5-methyl-2-(methylamino)quinazolin-4-yl]amino}methyl)phenyl]nicotinamide;
4-chloro-N-[4-({[7-methyl-2-(methylamino)quinazolin-4-yl]amino}methyl)phenyl]benzamide;
4-chloro-N-[4-({[2-(dimethylamino)-7-methyl-quinazolin-4-yl]amino}methyl)phenyl]benzamide;
4-chloro-N-{4-[({7-methyl-2-[(2-pyridin-2-ylethyl)amino]quinazolin-4-yl}amino)methyl]phenyl}benzamide;
N-(4-{[(2-azepan-1-yl-7-methylquinazolin-4-yl)amino]methyl}phenyl)-4-chlorobenzamide;
N-[4-({[2-(dimethylamino)-7-methylquinazolin-4-yl]amino}methyl)phenyl]-4-fluorobenzamide;
4-fluoro-N-[4-({[7-methyl-2-(4-pyridin-2-ylpiperazin-1-yl)quinazolin-4-yl]amino}methyl)phenyl]benzamide;
N-{4-[({2-[[3-(dimethylamino)propyl](methyl)amino]-7-methylquinazolin-4-yl}amino)methyl]phenyl}-4-fluorobenzamide;
N-(4-{[(2-azetidin-1-yl-7-methylquinazolin-4-yl)amino]methyl}phenyl)-4-fluorobenzamide;
N-{4-[({2-[benzyl(methyl)amino]-7-methylquinazolin-4-yl}amino)methyl]phenyl}-4-fluorobenzamide;
4-fluoro-N-[4-({[7-methyl-2-(4-methylpiperazin-1-yl)quinazolin-4-yl]amino}methyl)phenyl]benzamide;
4-fluoro-N-{4-[({2-[(2S)-2-(methoxy ethyl)pyrrolidin-1-yl]-7-methylquinazolin-4-yl}amino)methyl]phenyl}benzamide;
4-fluoro-N-(4-{[(7-methyl-2-piperidin-1-ylquinazolin-4-yl)amino]methyl}phenyl)benzamide;
4-fluoro-N-(4-{[(7-methyl-2-morpholin-4-ylquinazolin-4-yl)amino]methyl}phenyl)benzamide;
4-fluoro-N-(4-{[(7-methyl-2-piperazin-1-ylquinazolin-4-yl)amino]methyl}phenyl)benzamide;
4-fluoro-N-(4-{[(7-methyl-2-pyrrolidin-1-ylquinazolin-4-yl)amino]methyl}phenyl)benzamide;
N-{4-[({2-[ethyl(methyl)amino]-7-methylquinazolin-4-yl}amino)methyl]phenyl}-4-fluorobenzamide;
N-[4-({[2-(diethylamino)-7-methylquinazolin-4-yl]amino}methyl)phenyl]-4-fluorobenzamide;
4-fluoro-N-[4-({[7-methyl-2-(4-phenylpiperazin-1-yl)quinazolin-4-yl]amino}methyl)phenyl]benzamide;
4-fluoro-N-{4-[({7-methyl-2-[4-(2-oxo-2-pyrrolidin-1-ylethyl)piperazin-1-yl]quinazolin-4-yl}amino)methyl]phenyl}benzamide;
4-fluoro-N-{4-[({2-[(2-hydroxyethyl)(methyl)amino]-7-methylquinazolin-4-yl}amino)methyl]phenyl}benzamide;
N-{4-[({2-[4-(1,3-benzodioxol-5-ylmethyl)piperazin-1-yl]-7-methylquinazolin-4-yl}amino)methyl]phenyl}-4-fluorobenzamide;
4-fluoro-N-[4-({[7-methyl-2-(4-pyrimidin-2-ylpiperazin-1-yl)quinazolin-4-yl]amino}methyl)phenyl]benzamide;
4-fluoro-N-[4-({[2-(4-formylpiperazin-1-yl)-7-methylquinazolin-4-yl]amino}methyl)phenyl]benzamide;
Ethyl 4-[4-({4-[(4-fluorobenzoyl)amino]benzyl}amino)-7-methylquinazolin-2-yl]piperazine-1-carboxylate;
4-fluoro-N-(4-{[(2-{4-[2-(isopropylamino)-2-oxoethyl]piperazin-1-yl}-7-methylquinazolin-4-yl)amino]methyl}phenyl)benzamide;
4-fluoro-N-{4-[({2-[(2-methoxyethyl)(methyl)amino]-7-methylquinazolin-4-yl}amino)methyl]phenyl}benzamide;
4-fluoro-N-{4-[({2-[(2-furylmethyl)(methyl)amino]-7-methylquinazolin-4-yl}amino)methyl]phenyl}benzamide;
4-fluoro-N-{4-[({7-methyl-2-[methyl(2-pyridin-2-ylethyl)amino]quinazolin-4-yl}amino)methyl]phenyl}benzamide;
N-[4-({[2-(4-acetyl-1,4-diazepan-1-yl)-7-methylquinazolin-4-yl]amino}methyl)phenyl]-4-fluorobenzamide;
4-fluoro-N-{4-[({2-[2-(2-hydroxyethyl)piperidin-1-yl]-7-methylquinazolin-4-yl}amino)methyl]phenyl}benzamide;
4-fluoro-N-{4-[({7-methyl-2-[(3R)-3-methylpiperazin-1-yl]quinazolin-4-yl}amino)methyl]phenyl}benzamide;
4-fluoro-N-[4-({[7-methyl-2-(4-pyrrolidin-1-ylpiperidin-1-yl)quinazolin-4-yl]amino}methyl)phenyl]benzamide;
N-[4-({[2-(4-ethylpiperazin-1-yl)-7-methylquinazolin-4-yl]amino}methyl)phenyl]-4-fluorobenzamide;
4-fluoro-N-{4-[({7-methyl-2-[(2S)-2-(pyrrolidin-1-ylmethyl)pyrrolidin-1-yl]quinazolin-4-yl}amino)methyl]phenyl}benzamide;
4-fluoro-N-(4-{[(7-methyl-2-{[3-(4-methylpiperazin-1-yl)propyl]amino}quinazolin-4-yl)amino]methyl}phenyl)benzamide;
4-fluoro-N-[4-({[7-methyl-2-(methylamino)quinazolin-4-yl]amino}methyl)phenyl]benzamide;
4-fluoro-N-[4-({[7-methyl-2-(propylamino)quinazolin-4-yl]amino}methyl)phenyl]benzamide;
4-fluoro-N-{4-[({7-methyl-2-[(2-pyridin-2-ylethyl)amino]quinazolin-4-yl}amino)methyl]phenyl}benzamide;
N-{4-[({2-[(1-benzylpiperidin-4-yl)amino]-7-methylquinazolin-4-yl}amino)methyl]phenyl}-4-fluorobenzamide; and
4-fluoro-N-{4-[({7-methyl-2-[(3S)-3-methylpiperazin-1-yl]quinazolin-4-yl}amino)methyl]phenyl}benzamide.

In yet another aspect, the present invention provides a compound of formula I, or an enantiomer, diastereomer, tautomer, or pharmaceutically acceptable salt or solvate thereof, wherein the compound of formula I is selected from the group consisting of:
N-(4-{[(2-azepan-1-yl-7-methylquinazolin-4-yl)amino]methyl}phenyl)-4-fluorobenzamide;
N-[4-({[2-(3,3-dimethylpiperazin-1-yl)-7-methylquinazolin-4-yl]amino}methyl)phenyl]-4-fluorobenzamide;
4-fluoro-N-{4-[({7-methyl-2-[(2-pyrrolidin-1-ylethyl)amino]quinazolin-4-yl}amino)methyl]phenyl}benzamide;
4-bromo-N-[4-({[7-methyl-2-(methylamino)quinazolin-4-yl]amino}methyl)phenyl]benzamide;
4-bromo-N-[4-({[2-(dimethylamino)-7-methylquinazolin-4-yl]amino}methyl)phenyl]benzamide;
6-methyl-N-[4-({[7-methyl-2-(methylamino)quinazolin-4-yl]amino}methyl)phenyl]nicotinamide;
N-[4-({[2-(dimethylamino)-7-methylquinazolin-4-yl]amino}methyl)phenyl]-6-methylnicotinamide;
6-chloro-N-[4-({[2-(dimethylamino)-7-methylquinazolin-4-yl]amino}methyl)phenyl]nicotinamide;
N-[4-({[2-(dimethylamino)-7-methylquinazolin-4-yl]amino}methyl)phenyl]-1-methylpiperidine-4-carboxamide;
N-[4-({[2-(dimethylamino)-7-methylquinazolin-4-yl]amino}methyl)phenyl]-1-isobutylpiperidine-4-carboxamide;
1-cyclohexyl-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(2-furylmethyl)piperidine-4-carboxamide;
N-[4-({[2-(dimethylamino)-7-methylquinazolin-4-yl]amino}methyl)phenyl]-1-(4-methylbenzyl)piperidine-4-carboxamide;
N-[4-({[2-(dimethylamino)-7-methylquinazolin-4-yl]amino}methyl)phenyl]-1-(1H-imidazol-2-ylmethyl)piperidine-4-carboxamide;
1-butyl-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(4-methoxybenzyl)piperidine-4-carboxamide;
N-[4-({[2-(dimethylamino)-7-methylquinazolin-4-yl]amino}methyl)phenyl]-1-(4-fluorobenzyl)piperidine-4-carboxamide;
N-[4-({[2-(dimethylamino)-7-methylquinazolin-4-yl]amino}methyl)phenyl]-1-(2-fluorobenzyl)piperidine-4-carboxamide;
1-(4-chlorobenzyl)-N-[4-({[2-(dimethylamino)-7-methylquinazolin-4-yl]amino}methyl)phenyl]piperidine-4-carboxamide;
1-(2,4-difluorobenzyl)-N-[4-({[2-(dimethylamino)-7-methylquinazolin-4-yl]amino}methyl)phenyl]piperidine-4-carboxamide;
1-(3,4-difluorobenzyl)-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-[4-(trifluoromethyl)benzyl]piperidine-4-carboxamide;
N-[4-({[2-(dimethylamino)-7-methylquinazolin-4-yl]amino}methyl)phenyl]-1-(pyridin-4-ylmethyl)piperidine-4-carboxamide;
1-(2-chloro-4-fluorobenzyl)-N-[4-({[2-(dimethylamino)-7-methylquinazolin-4-yl]amino}methyl)phenyl]piperidine-4-carboxamide;
1-[(6-chloropyridin-3-yl)methyl]-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(2,4,6-trifluorobenzyl)piperidine-4-carboxamide;
N-[4-({[2-(dimethylamino)-7-methylquinazolin-4-yl]amino}methyl)phenyl]-1-(3-fluorobenzyl)piperidine-4-carboxamide;
1-(2,5-difluorobenzyl)-N-[4-({[2-(dimethylamino)-7-methylquinazolin-4-yl]amino}methyl)phenyl]piperidine-4-carboxamide;
1-(4-chloro-3-fluorobenzyl)-N-[4-({[2-(dimethylamino)-7-methylquinazolin-4-yl]amino}methyl)phenyl]piperidine-4-carboxamide;
6-chloro-N-[4-({[7-methyl-2-(methylamino)quinazolin-4-yl]amino}methyl)phenyl]nicotinamide;
4-bromo-N-[4-({[8-methyl-2-(methylamino)quinazolin-4-yl]amino}methyl)phenyl]benzamide-(preferably TFA salt);
4-cyano-N-[4-({[8-methyl-2-(methylamino)quinazolin-4-yl]amino}methyl)phenyl]benzamide-(preferably TFA salt);
N-[4-((1S)-1-{[2-(dimethylamino)-7-methylquinazolin-4-yl]amino}ethyl)phenyl]piperidine-4-carboxamide;
1-(3,4-difluorobenzyl)-N-[4-((1S)-1-{[2-(dimethylamino)-7-methylquinazolin-4-yl]amino}ethyl)phenyl]piperidine-4-carboxamide;
(S)-6-chloro-N-(4-(1-(2-(dimethylamino)-7-methylquinazolin-4-ylamino)ethyl)phenyl)nicotinamide;
(R)-6-chloro-N-(4-(1-(2-(dimethylamino)-7-methylquinazolin-4-ylamino)ethyl)phenyl)nicotinamide;
(S)—N-(4-(1-(2-(dimethylamino)-7-methylquinazolin-4-ylamino)ethyl)phenyl)-4-fluorobenzamide;
(R)—N-(4-(1-(2-(dimethylamino)-7-methylquinazolin-4-ylamino)ethyl)phenyl)-4-fluorobenzamide;
(S)-6-chloro-N-(4-(1-(2-(dimethylamino)-8-methylquinazolin-4-ylamino)ethyl)phenyl)nicotinamide;
(S)-6-chloro-N-(4-(1-(2-(dimethylamino)-6-methylquinazolin-4-ylamino)ethyl)phenyl)nicotinamide;
(S)-4-fluoro-N-(4-(1-(8-methyl-2-(methylamino)quinazolin-4-ylamino)ethyl)phenyl)benzamide; and
(R)-4-fluoro-N-(4-(1-(8-methyl-2-(methylamino)quinazolin-4-ylamino)ethyl)phenyl)benzamide.

In a further aspect, the present invention provides a pharmaceutical composition comprising at least one compound as described hereinabove, and a pharmaceutically-acceptable carrier or diluent. In certain embodiments, the pharmaceutical composition of the present invention may further comprise at least one other anti-cancer agent or cytotoxic agent. In some embodiments, the other anti-cancer or cytotoxic agent is selected from the group consisting of 5-FU, leucovorin, irinotecan, bevacizumab, cetuximab, intraarterial floxuridine, oxaliplatin, gefitinib, and fluorouracil.

In another aspect, the present invention provides a method of inhibiting beta-catenin/Tcf-4 pathway comprising administering to a mammalian species in need thereof an effective amount of at least one compound as described hereinabove.

In yet another aspect, the present invention provides a method for treating a condition or disorder comprising administering to a mammalian species in need thereof a therapeutically effective amount of at least one compound as described hereinabove, wherein the condition or disorder is selected from the group consisting of proliferate diseases and cancers. In certain embodiments, the condition or disorder is colorectal cancer. The invention also includes use of a compound of the invention in the manufacture of a medicament for the treatment of a condition or disorder is selected from the group consisting of proliferate diseases and cancers, preferably colorectal cancer.

In yet another aspect, the present invention provides a method for treating a condition or disorder comprising administering to a mammalian species in need thereof a therapeutically effective amount of at least one compound as described hereinabove, in combination with at least one other anti-cancer or cytotoxic agent. In certain embodiments, said other anti-cancer or cytotoxic agent is selected from the group consisting of 5-FU, leucovorin, irinotecan, bevacizumab, cetuximab, intraarterial floxuridine, oxaliplatin, gefitinib, and fluorouracil.

In a further aspect, the present invention provides a method of inhibiting the transcription of a gene selected from the group consisting of c-myc, cyclin D1, BMP4, KLF4, DHRS9/DHRL, MDR-1, Axin2, GPR49, ROR1, TIMP2, ID2, MSX1, and CSF2, comprising administering to a mammalian species in need thereof an effective amount of at least one compound as described hereinabove.

In another aspect, the present invention provides a method for making a compound of formula I,

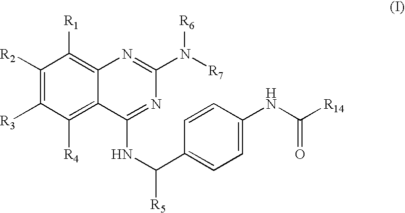

or an enantiomer, diastereomer, tautomer, or pharmaceutically acceptable salt or solvate thereof, comprising:
(a) reacting a compound of formula II,

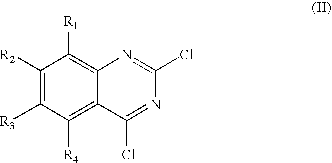

with an amine of formula III,

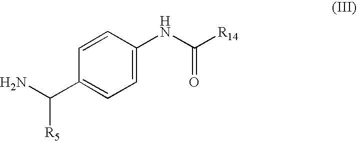

to provide a compound of formula IV; and

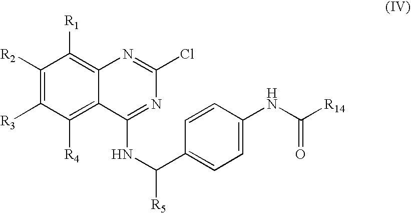

(b) further reacting the compound of formula IV with an amine of formula HNR₆R₇ to give the compound of formula I;
wherein the symbols of each of the above formulae have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
wherein: R₂ and R₃ together with the two contiguous carbon atoms to which R₂ and R₃ are bonded may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the present invention provides a method for making a compound of formula I,

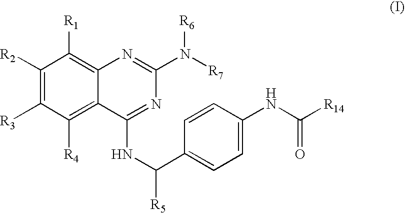

or an enantiomer, diastereomer, tautomer, or pharmaceutically acceptable salt or solvate thereof, comprising:
(a) reacting a compound of formula II,

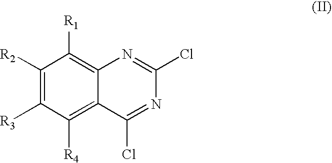

with an amine of formula V,

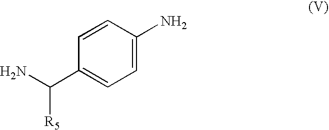

to provide a compound of formula VI;

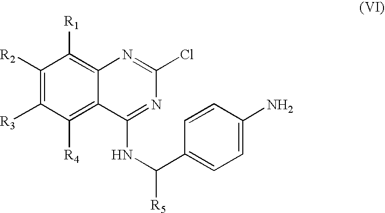

(b) reacting the compound of formula VI with an acid chloride of formula R₁₄(C═O)Cl, or an acid of formula of R₁₄C(═O)OH, to give a compound of formula IV; and

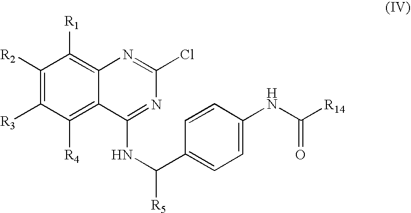

(c) further reacting the compound of formula IV with an amine of formula HNR₆R₇ to the compound of formula I;
wherein the symbols of each of the above formulae have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
wherein: R₂ and R₃ with the two contiguous carbon atoms to which R₂ and R₃ are bonded together may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
R_{b}, R_{c} and R_{d} are independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

In yet another aspect, the present invention provides a compound of formula I prepared according to the methods as described hereinabove.

### FURTHER DESCRIPTION OF THE INVENTION

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted.

The term “cycloalkyl” refers to a fully saturated cyclic hydrocarbon group containing from 1 to 4 rings and 3 to 8 carbons per ring. Exemplary such groups include cyclopropyl, cyclobutyl, cyclopentyl, cyclohexyl, cycloheptyl, etc. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The terms “halogen” or “halo” refer to chlorine, bromine, fluorine or iodine.

The term “carbocyclic” refers to aromatic or non-aromatic 3 to 7 membered monocyclic and 7 to 11 membered bicyclic groups, in which all atoms of the ring or rings are carbon atoms. “Substituted carbocyclic” refers to a carbocyclic group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurance of Rₐ is independently hydrogen, alkyl, cycloalkyl, alkenyl, cycloalkenyl, alkynyl, heterocycle, or aryl; each occurance of R_{b}, R_{c} and R_{d} is independently hydrogen, alkyl, cycloalkyl, heterocycle, aryl, or said R_{b} and R_{c} together with the N to which they are bonded optionally form a heterocycle; and each occurance of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, 3^{rd} edition, Wiley, N.Y. (1999).

Unless otherwise indicated, any heteroatom with unsatisfied valences is assumed to have hydrogen atoms sufficient to satisfy the valences.

The compounds of formulae I through VI form salts which are also within the scope of this invention. Reference to a compound of formulae I through VI herein is understood to include reference to salts thereof, unless otherwise indicated. The term “salt(s)”, as employed herein, denotes acidic and/or basic salts formed with inorganic and/or organic acids and bases. In addition, when a compound of formulae I through VI contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formulae I through VI may be formed, for example, by reacting a compound I with an amount of acid or base, such as an equivalent amount, in a medium such as one in which the salt precipitates or in an aqueous medium followed by lyophilization.

The compounds of formulae I through VI which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of formulae I through VI which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the formulae I through VI, or a salt and/or solvate thereof. Solvates of the compounds of formulae I through VI include, for example, hydrates.

Compounds of the formulae I through VI, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the formulae I through VI are, subsequent to their preparation, preferably isolated and purified to obtain a composition containing an amount by weight equal to or greater than 99% formulae I through VI compound (“substantially pure” compound I), which is then used or formulated as described herein. Such “substantially pure” compounds of the formulae I through VI are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

### Abbreviations

ACN acetonitrile
ArCHO aryl-aldehyde, such as benzaldehyde
Axin2 Axin-related protein
BMP4 bone morphogenetic protein 4
Boc t-butoxycarbonyl
CCD charge-coupled device
CH₂Cl₂ dichloromethane
CH₂O formaldehyde
CHCl₃ chloroform
c-myc v-myc myelocytomatosis viral oncogene homolog (avian)
CO carbon monoxide
Conc. concentrated
CSF2 colony stimulating factor 2 (granulocyte-macrophage)
CuI copper iodide
cyclin D1 a member of the cyclin family
DHRS9/DHRL dehydrogenase/reductase (SDR family) member 9

### DMEM Dulbecco's Modified Eagle Media

### DMF Dimethylformamide

EDCl 1-ethyl-3(3-dimethyl aminopropyl)carbodiimide hydrochloride
Et₃N or NEt₃ triethyl amine
EtI ethyl iodide
EtOAc ethyl acetate
EtOH ethyl alcohol
FBS fetal bovine serum
GPR49 leucine-rich repeat-containing G protein-coupled receptor 5 (a/k/a LGR5)
HCl hydrochloric acid
HCO₂Na sodium formate
HF hydrogen floride
HOBT 1-hydroxybenzotriazole
HPLC High performance liquid chromatography
ID2 inhibitor of DNA binding 2, dominant negative helix-loop-helix protein
K₂CO₃ potassium carbonate
KLF4 Kruppel-like factor 4 (gut)
MDR-1 ATP-binding cassette, sub-family B (MDR/TAP), member 1
Me₂CO propan-2-one

### MeOH Methanol

MgSO₄ magnesium sulfate
MSX1 msh homeo box homolog 1 (Drosophila)
NaBH₃CN sodium cyanotrihydroborate
NaBH₄ sodium borohydride
NaHCO₃ Sodium bicarbonate
NH₄OH ammonim hydroxide
PCl₅ phosphorus pentachloride
Pd(PPh₃)₄ tetrakis(triphenylphosphine)palladium(0)
Pd—C/H₂ palladium-carbon/hydrogen
PdCl₂(PPh₃)₂ dichlorobis(triphenylphosphine)palladium(II)
pGL3 Luciferase Reporter Vector

### Ph₂O Benzophenone

POCl₃ phosphorus oxytrichloride
ROR1 receptor tyrosine kinase-like orphan receptor 1
RT or rt room temperature
SnCl₂ tin chloride
SV-40 Simian vacuolating virus 40
SV40-Luc a luciferase reporter gene driven by SV40 promoter
SV40-R-Luc a renilla luciferase reporter gene driven by SV40 promoter
SW480 a human colon cancer cell line
TEA triethyl amine
TFA trifluoroacetic acid

### THF Tetrahydrofuran

TIMP2 tissue inhibitor of metalloproteinase 2
TLC thin layer chormoatograph
Zn(CN)₂ zinc cyanide
ZnCl₂ zinc chloride

### Methods of Preparation

The compounds of the present invention can be prepared using the methods described below, together with synthetic methods known one skilled in the art of organic synthesis, or variations thereon. The reactions are performed in solvents appropriate to the reagents and materials employed and are suitable for transformations being effected. The starting materials for the examples contained herein are either commercially available or are readily prepared by standard methods from known materials. For example, the following reactions are illustrations but not limitations of the preparation of some of the starting materials and examples used herein.

Compounds of formula I can be prepared starting from appropriately substituted 2-aminobenzoic acid derivatives as outlined in Scheme 1. The appropriately substituted 2-aminobenzoic acids 1 can react with urea, preferably at elevated temperatures such as 180-220° C. and in the presence of hydrochloric acid (HCl) to give compound 2. Compound 2 can further react with at least one chlorinating agent such as phosphorus oxytrichloride (POCl₃) and/or phosphorus pentachloride (PCl₅) to provide dichloro derivatives II. As shown in Path A, dichloro derivatives II can react with an amine of formula III in the presence of a base, such as triethyl amine (Et₃N), and in an organic solvent such as chloroform (CHCl₃) to afford compound IV. Compound IV can further react with an amine of formula HNR₆R₇ in the presence of a base, such as triethyl amine, to provide compound I.

Alternatively, as shown in Path B, dichloro derivatives II can react with an amine of formula V to afford compound VI, which in turn can react with an acid chloride of formula R₁₄(C═O)Cl in the presense of a base, or an acid of formula R₁₄(C═O)OH in the presence of an amide coupling agent and a base, to give compound IV. Compound IV can further react with an amine of formula HNR₆R₇ in the presence of a base, such as triethyl amine, to provide compound I.

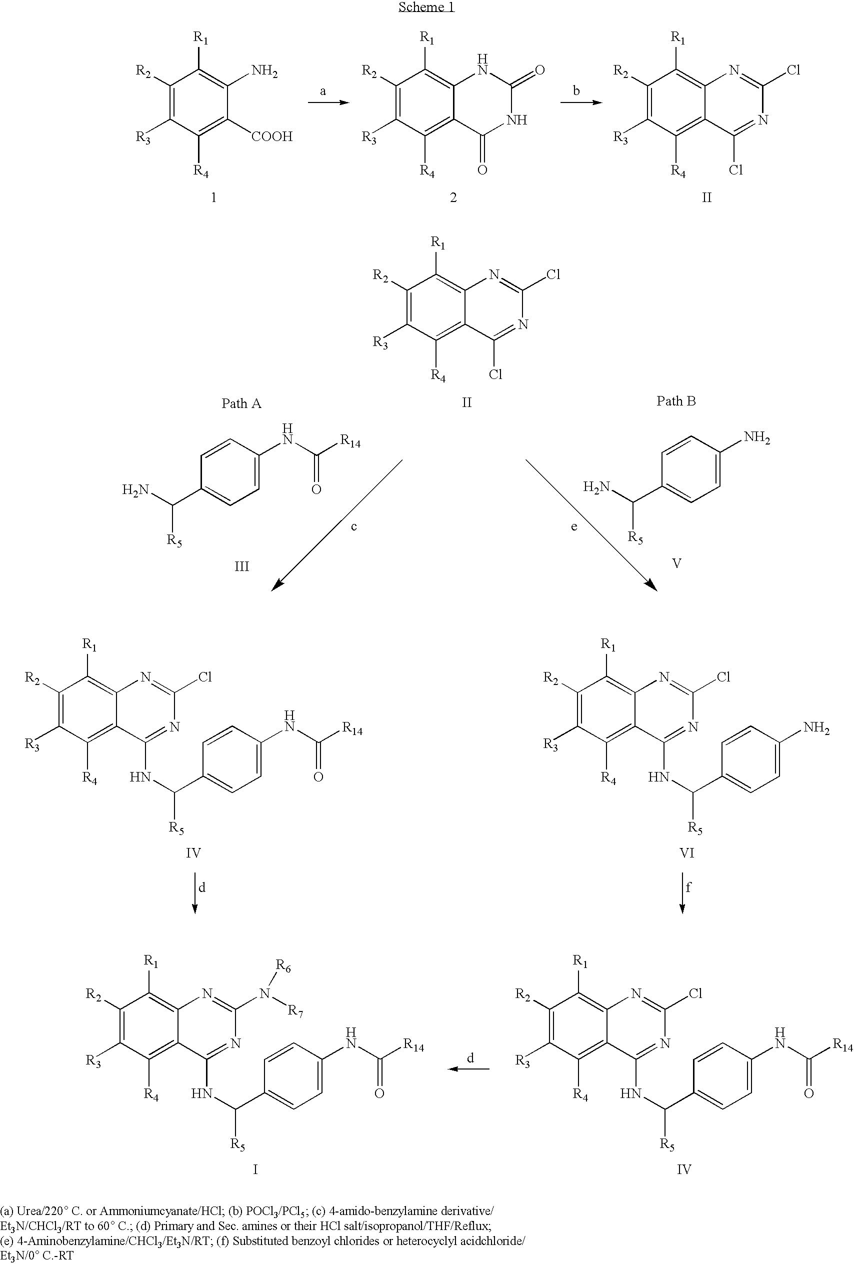

Dichloro derivatives II can also be prepared starting from aniline 8 as outlined in Scheme 2. Aniline 8 can react with ethyl isothioscyanatoformate to give intermediate 9, which can further react with ethyl iodide (EtI) in the presense of a base, such as potassium carbonate (K₂CO₃), to provide intermediate 10. Intermediate 10 can undergo cyclization at elevated temperature to afford compound 11, which can be further transformed to compound 2 under acidic condition. Finally, compound 2 can react with at least one chlorinating agent such as POCl₃/PCl₅ to provide dichloro derivatives II.

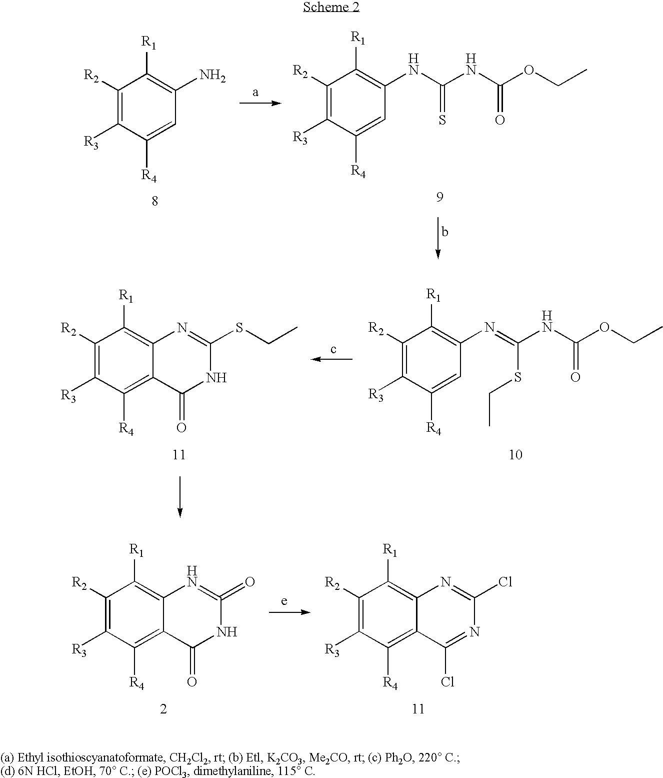

According to Scheme 3, the compound of formula I having amino-substituted structures (e.g., compounds 13, 14-1, 14-2, and 15) can be prepared starting from their respective nitro derivatives 12. The nitro derivatives 12 can be reduced to amino-substituted compounds 13 using palladium-carbon/hydrogen (Pd—C/H₂) or tin chloride (SnCl₂). The resulting compounds 13 can used to prepare N,N-dialkyl derivatives 14-1 or 14-2 via reductive amination. Compounds 13 can also react with acidchlorides to obtain compound 15.

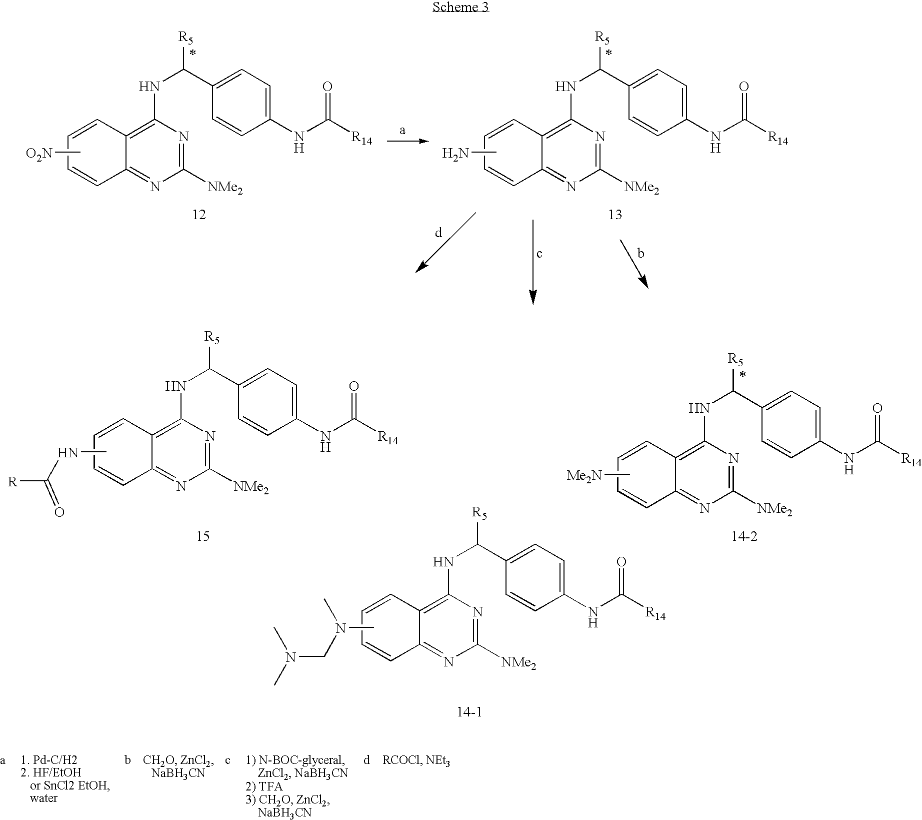

The compound of formula I having a piperidine moiety (e.g., compounds 18, 19, 20 and 21) can be prepared staring from appropriately substituted 2,4-dichloro derivatives 11 and t-Boc-protected compound 16 according to Scheme 4 and Scheme 5. Intermediate 17 can be aminated using primary or secondary amines. The Boc-group can be removed by using an acid, such as trifluoroacetic acid (TFA), and various R₁₃ groups can be introduced. As a non-limiting example, various alkyl and benzyl substituents can be introduced at the nitrogen of the piperidine moiety, by a reductive amination process.

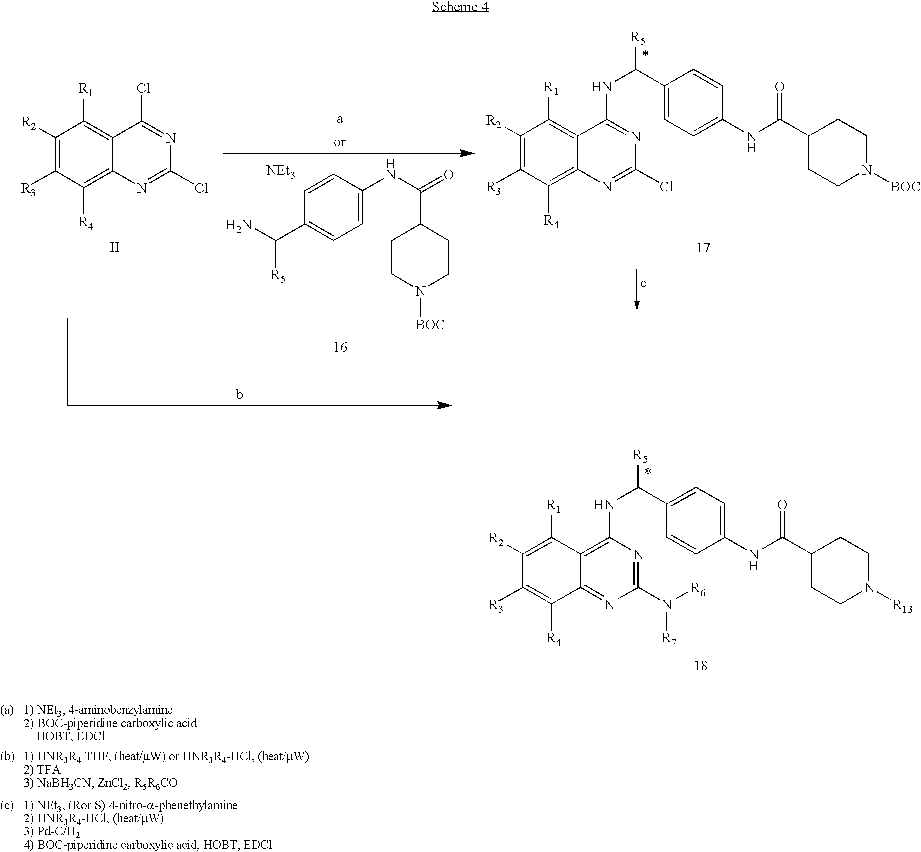

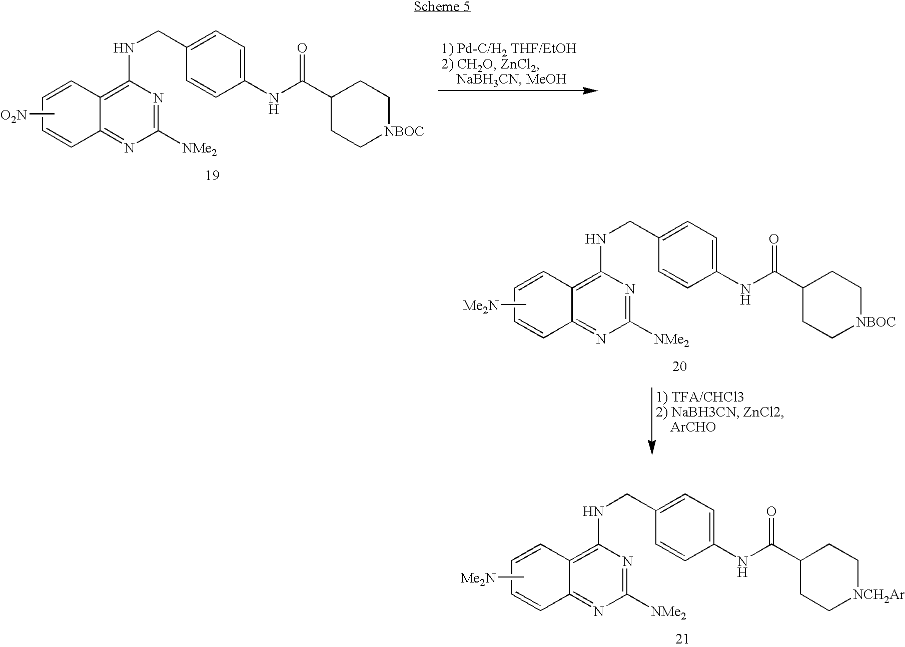

### EXAMPLES

### Procedure A

### Step 1

To a stirred solution of 122 mg (1 mmol) 4-aminobenzylamine and 202 mg (2 mmol) triethylamine in 5 mL CHCl₃ at rt was added 1 mmol of the appropriately substituted 2,4-dichloroquinazoline was added and stirred for a minimum of 3 hours. After thin layer chormoatograph (TLC) showed the complete disappearance of the 2,4-dichloroquinazoline, 1 mmol of aroylchloride in 1 mL CHCl₃ was added slowly. The reaction mixture was allowed to stir overnight. At the end the reaction mixture was quenched with 50 mL of CHCl₃ and 15 mL water. The reaction mixture was washed well with water and the chloroform layer was dried over magnesium sulfate (MgSO₄). After removal of MgSO₄ by filtration and evaporation of solvents the crude product was purified by column chromatography with hexane/CH₂Cl₂/triethyl amine (TEA) to give the 2-chloroquinazolines in yields between 50-95%.

### Procedure A

### Step 2

The appropriately substituted 2-chloro quinazoline derivatives (1 mmol) obtained by the Procedure A, step 1 was taken up either in a sealed tube or in round bottom flask and was suspended in 5 mL THF or dioxane. (If the reactant amine was mono methylamine or dimethylamine sealed tube was used and for other amines round bottom flask can be used.) The appropriate amine was added and the mixture was heated over 16 h to 100° C. or alternatively heated for 40 min to 120° C. using microwave. After reaction was completed the solvents were removed in vacuo and the crude compound purified by silica-gel column chromatography by using CH₂Cl₂/MeOH/NH₃-mixtures as eluent to give the diamino quinazolines in yields between 65-95%.

### Example 1

### Preparation of 5-fluoro-2-methyl-N-[4-({[2-(methylamino)quinazolin-4-yl]amino}methyl)phenyl]benzamide

5-fluoro-2-methyl-N-[4-({[2-(methylamino)quinazolin-4-yl]amino}methyl)phenyl]benzamide was prepared starting from 2,4-dichloroquinazoline, 4-aminobenzylamine and 2-methyl-4-fluorobenzoyl chloride by following the procedure A (step 1). The intermediate product from the step 1 was aminated using monomethylamine to yield the final product. Starting from 2,4-dichloroquinazoline (0.140 g, 0.7 mmol), 25 mg (Yield, 18%) of the final product was isolated. MS (ESI) m/z 416.2.

### Example 2

### Preparation of 2-(benzyloxy)-N-[4-({[2-(methylamino)quinazolin-4-yl]amino}methyl)phenyl]acetamide

2-(Benzyloxy)-N-[4-({[2-(methylamino)quinazolin-4-yl]amino}methyl)phenyl]acetamide was prepared starting from 2,4-dichloroquinazoline, 4-aminobenzylamine and benzyloxy-acetyl chloride by following the procedure A (step 1). The intermediate product from the step 1 was aminated using monomethylamine to yield the final product. Starting from 2,4-dichloroquinazoline, (0.20 g, 1.0 mmol), 50 mg (Yield, 25%) of the final product was isolated. MS (ESI) m/z 428.2.

### Example 3

### Preparation of 6-chloro-N-[4-({[2-(methylamino)quinazolin-4-yl]amino}methyl)phenyl]nicotinamide

6-chloro-N-[4-({[2-(methylamino)quinazolin-4-yl]amino}methyl)phenyl]nicotinamide was prepared starting from 2,4-dichloroquinazoline, 4-aminobenzylamine and 6-chloronicotinoyl chloride by following the procedure A (step 1). The intermediate product from the step 1 was aminated using monomethylamine to yield the final product. Starting from 2,4-dichloroquinazoline, (0.15 g, 0.75 mmol) 140 mg (Yield, 95%) of the final product was isolated. MS (ESI) m/z 419.1.

### Example 4

### Preparation of 2-chloro-N-[4-({[2-(methylamino)quinazolin-4-yl]amino}methyl)phenyl]isonicotinamide

2-chloro-N-[4-({[2-(methylamino)quinazolin-4-yl]amino}methyl)phenyl]isonicotinamide was prepared starting from 2,4-dichloroquinazoline, 4-aminobenzylamine and 2-chloro-isonicotinoyl chloride by following the procedure A (step 1). The intermediate product from the step 1 was aminated using monomethylamine to yield the final product. Starting from 2,4-dichloroquinazoline, (0.18 g, 0.9 mmol), 40 mg (Yield, 24%) of the final product was isolated. MS (ESI) m/z 419.8.

### Example 5

### Preparation of N-[4-({[2-(methylamino)quinazolin-4-yl]amino}methyl)phenyl]quinoline-2-carboxamide

N-[4-({[2-(methylamino)quinazolin-4-yl]amino}methyl)phenyl]quinoline-2-carboxamide was prepared starting from 2,4-dichloroquinazoline, 4-aminobenzylamine and 2-chloro-isoquinoline-3-carbonyl chloride by following the procedure A (step 1). The intermediate product from the step 1 was aminated using monomethylamine to yield the final product. Starting from 2,4-dichloroquinazoline, (0.20 g, 1.0 mmol), 16 mg (18%) of the final product was isolated. MS (ESI) 436.

### Example 6

### Preparation of 2-chloro-5-fluoro-N-[4-({[2-(methylamino)quinazolin-4-yl]amino}methyl)phenyl]benzamide

2-chloro-5-fluoro-N-[4-({[2-(methylamino)quinazolin-4-yl]amino}methyl)phenyl]benzamide was prepared starting from 2,4-dichloroquinazoline, 4-aminobenzylamine and 2-chloro-5-fluorobenzoylchloride by following the procedure A (step 1). The intermediate product from the step 1 was aminated using monomethylamine to yield the final product. Starting from 2,4-dichloroquinazoline, (0.20 g, 1.0 mmol), 18 mg (Yield, 9%) of the final product was isolated. MS (ESI) m/z 436.5.

### Example 7

### Preparation of 2-chloro-N-[4-({[2-(methylamino)quinazolin-4-yl]amino}methyl)phenyl]nicotinamide

2-chloro-N-[4-({[2-(methylamino)quinazolin-4-yl]amino}methyl)phenyl]nicotinamide was prepared starting from 2,4-dichloroquinazoline, 4-aminobenzylamine and 2-chloronicotinoyl chloride by following the procedure A (step 1). The intermediate product from the step 1 was aminated using monomethylamine to yield the final product. Starting from 2,4-dichloroquinazoline, (0.20 g, 1.0 mmol), 21 mg (Yield, 11% of the final product was isolated. MS (ESI) m/z 419.

### Example 8

### Preparation of 2,6-dichloro-5-fluoro-N-[4-({[2-(methylamino)quinazolin-4-yl]amino}methyl)phenyl]nicotinamide

2,6-dichloro-5-fluoro-N-[4-({[2-(methylamino)quinazolin-4-yl]amino}methyl)phenyl]nicotinamide was prepared starting from 2,4-dichloroquinazoline, 4-aminobenzylamine and 2,6-dichloro-5-fluoro-nicotinoylchloride following the procedure A (step 1). The intermediate product from the step 1 was aminated using monomethylamine to yield the final product. Starting from 2,4-dichloroquinazoline, (0.20 g, 1.0 mmol), 150 mg (Yield, 58%) of the final product was isolated. MS (ESI) m/z 471.

### Example 9

### Preparation of 6-chloro-N-[4-({[6,7-dimethoxy-2-(methylamino)quinazolin-4-yl]amino}methyl)phenyl]nicotinamide

6-chloro-N-[4-({[6,7-dimethoxy-2-(methylamino)quinazolin-4-yl]amino}methyl)phenyl]nicotinamide was prepared starting from 2,4-dichloro-6,7-dimethoxyquinazoline, 4-aminobenzylamine and 6-chloronicotinoyl chloride by following the procedure A (step 1). The intermediate product from the step 1 was aminated using monomethylamine to yield the final product. Starting from 2,4-dichloro-6,7-dimethoxyquinazoline, (0.20 g, 1.0 mmol), 71 mg (Yield, 36%) of the final product was isolated. MS (ESI) m/z 479.1.

### Example 10

### Preparation of 4-chloro-N-[4-({[2-(methylamino)quinazolin-4-yl]amino}methyl)phenyl]benzamide

4-chloro-N-[4-({[2-(methylamino)quinazolin-4-yl]amino}methyl)phenyl]benzamide was prepared starting from 2,4-dichloroquinazoline, 4-aminobenzylamine and 4-chlorobenzoyl chloride by following the procedure A (step 1). The intermediate product from the step 1 was aminated using monomethylamine to yield the final product. Starting from 2,4-dichloroquinazoline, (0.10 g, 0.5 mmol), 20 mg (Yield, 41%) of the final product was isolated. MS (ESI) m/z 418.1.

### Example 11

### Preparation of 3-chloro-N-[4-({[2-(methylamino)quinazolin-4-yl]amino}methyl)phenyl]benzamide

3-chloro-N-[4-({[2-(methylamino)quinazolin-4-yl]amino}methyl)phenyl]benzamide was prepared starting from 2,4-dichloroquinazoline, 4-aminobenzylamine and 3-chlorobenzoyl chloride by following the procedure A (step 1). The intermediate product from the step 1 was aminated using monomethylamine to yield the final product. Starting from 2,4-dichloroquinazoline, (0.10 g, 0.5 mmol) 30 mg (Yield, 30%) of the final product was isolated. MS (ESI) m/z 418.1.

### Example 12

### Preparation of 4-methyl-N-[4-({[2-(methylamino)quinazolin-4-yl]amino}methyl)phenyl]benzamide

4-methyl-N-[4-({[2-(methylamino)quinazolin-4-yl]amino}methyl)phenyl]benzamide was prepared starting from 2,4-dichloroquinazoline, 4-aminobenzylamine and 4-methyl-benzoylchloride by following the procedure A (step 1). The intermediate product from the step 1 was aminated using monomethylamine to yield the final product. Starting from of 2,4-dichloroquinazoline, (0.50 g, 2.5 mmol) of 200 mg (Yield, 40%) of the final product was isolated. MS (ESI) m/z 398.2.

### Example 13

### Preparation of 4-fluoro-N-[4-({[2-(methylamino)quinazolin-4-yl]amino}methyl)phenyl]benzamide

4-fluoro-N-[4-({[2-(methylamino)quinazolin-4-yl]amino}methyl)phenyl]benzamide was prepared starting from 2,4-dichloroquinazoline, 4-aminobenzylamine and 4-fluorobenzoylchloride by following the procedure A (step 1). The intermediate product from the step 1 was aminated using monomethylamine to yield the final product. Starting from 2,4-dichloroquinazoline, (0.38 g, 1.9 mmol), 010 mg (Yield, 55%) of the final product was isolated. MS (ESI) m/z 402.1.

### Example 14

### Preparation of 4-chloro-2-fluoro-N-[4-({[2-(methylamino)quinazolin-4-yl]amino}methyl)phenyl]benzamide

4-chloro-2-fluoro-N-[4-({[2-(methylamino)quinazolin-4-yl]amino}methyl)phenyl]benzamide was prepared starting from 2,4-dichloroquinazoline, 4-aminobenzylamine, and 2-fluoro-4-chlorobenzoylchloride by following the procedure A (step 1). The intermediate product from the step 1 was aminated using monomethylamine to yield the final product. Starting from 2,4-dichloroquinazoline, (0.20 g, 1.0 mmol), 70 mg (Yield, 63%) of the final product was isolated. MS (ESI) m/z 436.1.

### Example 15

### Preparation of N-[4-({[2-(methylamino)quinazolin-4-yl]amino}methyl)phenyl]biphenyl-4-carboxamide

N-[4-({[2-(methylamino)quinazolin-4-yl]amino}methyl)phenyl]biphenyl-4-carboxamide was prepared starting from 2,4-dichloroquinazoline, 4-aminobenzylamine and biphenyl-4-carbonyl chloride by following the procedure A (step 1). The intermediate product from the step 1 was aminated using monomethylamine to yield the final product. Starting from 2,4-dichloroquinazoline, (0.17 g, 0.85 mmol) 50 mg (Yield, 49%) of the final product was isolated. MS (ESI) m/z 460.2.

### Example 16

### Preparation of 2,4-dichloro-N-[4-({[2-(methylamino)quinazolin-4-yl]amino}methyl)phenyl]benzamide

2,4-dichloro-N-[4-({[2-(methylamino)quinazolin-4-yl]amino}methyl)phenyl]benzamide was prepared starting from 2,4-dichloroquinazoline, 4-aminobenzylamine and 2,4-dichlorobenzoyl chloride by following the procedure A (step 1). The intermediate product from the step 1 was aminated using monomethylamine to yield the final product. Starting from 2,4-dichloroquinazoline, (0.16 g, 0.8 mmol) 40 mg (Yield, 57%) of the final product was isolated. MS (ESI) m/z 452.2.

### Example 17

### Preparation of 4-fluoro-3-methyl-N-[4-({[2-(methylamino)quinazolin-4-yl]amino}methyl)phenyl]benzamide

4-fluoro-3-methyl-N-[4-({[2-(methylamino)quinazolin-4-yl]amino}methyl)phenyl]benzamide was prepared starting from 2,4-dichloroquinazoline, 4-aminobenzylamine and 3methyl-4-chloro benzoylchloride by following the procedure A (step 1). The intermediate product from the (step 1) was aminated using monomethylamine to yield the final product. Starting from 2,4-dichloroquinazoline, (0.155 g, 0.77 mmol) 50 mg Yield, 41%) of the final product was isolated. MS (ESI) m/z 416.

### Example 18

### Preparation of 4-chloro-N-[4-({[6-methyl-2-(methylamino)quinazolin-4-yl]amino}methyl)phenyl]benzamide

4-chloro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 4-chloro benzoylchloride by following the procedure A (step 1). The intermediate product from the (step 1) was aminated using monomethylamine to yield the final product. Starting from 6-methyl-2,4-dichloroquinazoline, (0.7 g, 3.5 mmol), 400 mg (Yield, 61%) of the final product was isolated. (ESI) m/z 432.3.

### Example 19

### Preparation of 2,4-dichloro-N-[4-({[6-methyl-2-(methylamino)quinazolin-4-yl]amino}methyl)phenyl]benzamide

2,4-dichloro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 2,4-dichloro benzoylchloride by following the procedure A (step 1). The intermediate product from the (step 1) was aminated using monomethylamine to yield the final product. Starting from 6-methyl-2,4-dichloroquinazoline, (0.21 g, 1.0 mmol) 40 mg (Yield, 50%) of the final product was isolated. MS (ESI) m/z 466.1.

### Example 20

### Preparation of N-[4-({[2-(methylamino)quinazolin-4-yl]amino}methyl)phenyl]-4-(trifluoromethyl)benzamide

N-[4-({[2-(methylamino)quinazolin-4-yl]amino}methyl)phenyl]-4-(trifluoromethyl)benzamide was prepared starting from 2,4-dichloroquinazoline, 4-aminobenzylamine and 4-trifluoromethyl benzoylchloride by following the procedure A (step 1). The intermediate product from the (step 1) was aminated using monomethylamine to yield the final product. Starting from 2,4-dichloroquinazoline, (0.19 g, 1.0 mmol) 50 mg (Yield, 43%) of the final product was isolated. MS (ESI) m/z 452.2.

### Example 21

### Preparation of 4-cyano-N-[4-({[2-(methylamino)quinazolin-4-yl]amino}methyl)phenyl]benzamide

4-cyano-N-[4-({[2-(methylamino)quinazolin-4-yl]amino}methyl)phenyl]benzamide was prepared starting from 2,4-dichloroquinazoline, 4-aminobenzylamine and 4-cyano-benzoylchloride by following the procedure A (step 1). The intermediate product from the (step 1) was aminated using monomethylamine to yield the final product. Starting from 2,4-dichloroquinazoline, (0.19 g, 1.0 mmol), 30 mg (Yield, 16%) of the final product was isolated. MS (ESI) m/z 409.

### Example 22

### Preparation of N-[4-({[2-(methylamino)quinazolin-4-yl]amino}methyl)phenyl]-4-(trifluoromethoxy)benzamide

N-[4-({[2-(methylamino)quinazolin-4-yl]amino}methyl)phenyl]-4-(trifluoromethoxy)benzamide was prepared starting from 2,4-dichloroquinazoline, 4-aminobenzylamine and 4-trifluoromethoxy-benzoylchloride by following the procedure A (step 1). The intermediate product from the (step 1) was aminated using monomethylamine to yield the final product. Starting from (0.19 g, 1.0 mmol) of 2,4-dichloroquinazoline, (0.19 g, 1.0 mmol), 30 mg (Yield, 31%) of the final product was isolated. MS (ESI) m/z 468.

### Example 23

### Preparation of 6-chloro-N-[4-({[6,8-dimethyl-2-(methylamino)quinazolin-4-yl]amino}methyl)phenyl]nicotinamide

6-chloro-N-[4-({[6,8-dimethyl-2-(methylamino)quinazolin-4-yl]amino}methyl)phenyl]nicotinamide was prepared starting from 6,8-dimethyl-2,4-dichloroquinazoline, 4-aminobenzylamine and 6-nicotinoyl chloride by following the procedure A (step 1). The intermediate product from the (step 1) was aminated using monomethylamine to yield the final product. Starting from 6,8-dimethyl-2,4-dichloroquinazoline, (0.226, 1.0 mmol), 100 mg Yield, 17%) of the final product was isolated. MS (ESI) m/z 447.

### Example 24

### Preparation of 4-fluoro-N-[4-({[6-methyl-2-(methylamino)quinazolin-4-yl]amino}methyl)phenyl]benzamide

4-fluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 4-fluorobenzoylchloride by following the procedure A (step 1). The intermediate product from the (step 1) was aminated using monomethylamine to yield the final product. Starting from (0.41 g, 2.0 mmol) of 6-methyl-2,4-dichloroquinazoline, 200 mg Yield, 58%) of the final product was isolated. MS (ESI) m/z 416.2.

### Example 25

### Preparation of 4-cyano-N-[4-({[6-methyl-2-(methylamino)quinazolin-4-yl]amino}methyl)phenyl]benzamide

4-cyano-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 4-cyanobenzoylchloride by following the procedure A (step 1). The intermediate product from the (step 1) was aminated using monomethylamine to yield the final product. Starting from 6-methyl-2,4-dichloroquinazoline, (0.41 g, 2.0 mmol) 250 mg (Yield, 59%) of the final product was isolated. MS (ESI) m/z 423.2.

### Example 26

### Preparation of 4-chloro-2-fluoro-N-[4-({[6-methyl-2-(methylamino)quinazolin-4-yl]amino}methyl)phenyl]benzamide

4-chloro-2-fluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 2-fluoro-4-chlorobenzoylchloride by following the procedure A (step 1). The intermediate product from the (step 1) was aminated using monomethylamine to yield the final product. Starting from 6-methyl-2,4-dichloroquinazoline, (0.41 g, 2.0 mmol), 100 mg (Yield, 24%) of the final product was isolated. MS (ESI) m/z 456.

### Example 27

### Preparation of N-[4-({[6-methyl-2-(methylamino)quinazolin-4-yl]amino}methyl)phenyl]-4-(trifluoromethyl)benzamide

N-[4-({[6-methyl-2-(methylamino)quinazolin-4-yl]amino}methyl)phenyl]-4-(trifluoromethyl)benzamide was prepared starting from 6-methyl-2,4-dichloroquinazoline, 4-aminobenzylamine and 4-trifluoromethyl-benzoylchloride by following the procedure A (step 1). The intermediate product from the (step 1) was aminated using monomethylamine to yield the final product. Starting from 6-methyl-2,4-dichloroquinazoline, (0.19 g, 0.90 mmol) 28 mg (Yield, 28%) of the final product was isolated. MS (ESI) m/z 466.1.

### Example 28

### Preparation of 2-chloro-4-fluoro-N-[4-({[6-methyl-2-(methylamino)quinazolin-4-yl]amino}methyl)phenyl]benzamide

2-chloro-4-fluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 2-chloro-4-fluorobenzoylchloride by following the procedure A (step 1). The intermediate product from the (step 1) was aminated using monomethylamine to yield the final product. Starting from 6-methyl-2,4-dichloroquinazoline, (0.41 g, 2.0 mmol), 150 mg (Yield, 44%) of the final product was isolated. MS (ESI) m/z 450.1.

### Example 29

### Preparation of 4-chloro-N-[4-({[6,8-dimethyl-2-(methylamino)quinazolin-4-yl]amino}methyl)phenyl]benzamide

4-chloro-N-[4-({[6,8-dimethyl-2-(methylamino)quinazolin-4-yl]amino}methyl)phenyl]benzamide was prepared starting from 6,8-dimethyl-2,4-dichloroquinazoline, 4-aminobenzylamine and 4-chlorobenzoylchloride by following the procedure A (step 1). The intermediate product from the (step 1) was aminated using monomethylamine to yield the final product. Starting from 6,8-dimethyl-2,4-dichloroquinazoline, (0.7 g, 3.0 mmol) 400 mg (Yield, 63%) of the final product was isolated. MS (ESI) m/z 446.2.

### Example 30

### Preparation of N-[4-({[6,8-dimethyl-2-(methylamino)quinazolin-4-yl]amino}methyl)phenyl]-4-fluorobenzamide

N-[4-({[6,8-dimethyl-2-(methylamino)quinazolin-4-yl]amino}methyl)phenyl]-4-fluorobenzamide was prepared starting from 6,8-dimethyl-2,4-dichloroquinazoline, 4-aminobenzylamine and 4-fluorobenzoylchloride by following the procedure A (step 1). The intermediate product from the (step 1) was aminated using monomethylamine to yield the final product. Starting from 6,8-dimethyl-2,4-dichloroquinazoline, (0.25 g, 1.0 mmol), 30 mg (Yield, 29%) of the final product was isolated. MS (ESI) m/z 430.1.

### Example 31

### Preparation of 6-chloro-N-[4-({[6-methoxy-2-(methylamino)quinazolin-4-yl]amino}methyl)phenyl]nicotinamide

6-chloro-N-[4-({[6-methoxy-2-(methylamino)quinazolin-4-yl]amino}methyl)phenyl]nicotinamide was prepared starting from 6-methoxy-2,4-dichloroquinazoline, 4-aminobenzylamine and 6-chloronicotinoyl chloride by following the procedure A (step 1). The intermediate product from the (step 1) was aminated using monomethylamine to yield the final product. Starting from 6-methoxy-2,4-dichloroquinazoline, (0.20 g, 0.88 mmol), 30 mg (Yield, 10%) of the final product was isolated. MS (ESI) m/z 449.2.

### Example 32

### Preparation of 3,4-difluoro-N-[4-({[6-methyl-2-(methylamino)quinazolin-4-yl]amino}methyl)phenyl]benzamide

3,4-difluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 3,4-difluorobenzoylchloride by following the procedure A (step 1). The intermediate product from the (step 1) was aminated using monomethylamine to yield the final product. Starting from 6-methyl-2,4-dichloroquinazoline, (0.25 g, 1.0 mmol), 40 mg (Yield, 34%) of the final product was isolated. MS (ESI) m/z 434.2.

### Example 33

### Preparation of 4-chloro-N-[4-({[6-methoxy-2-(methylamino)quinazolin-4-yl]amino}methyl)phenyl]benzamide

4-chloro-N-[4-({[6-methoxy-2-(methylamino)quinazolin-4-yl]amino}methyl)phenyl]benzamide was prepared starting from 6-methoxy-2,4-dichloroquinazoline, 4-aminobenzylamine and 4-chlorobenzoylchloride by following the procedure A (step 1). The intermediate product from the (step 1) was aminated using monomethylamine to yield the final product. Starting from 6-methoxy-2,4-dichloroquinazoline, (0.24 g, 1.0 mmol) 40 mg (Yield, 35%) of the final product was isolated. MS (ESI) m/z 448.1.

### Example 34

### Preparation of 2,4-difluoro-N-[4-({[6-methyl-2-(methylamino)quinazolin-4-yl]amino}methyl)phenyl]benzamide

2,4-difluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 2,4-difluorobenzoylchloride by following the procedure A (step 1). The intermediate product from the (step 1) was aminated using monomethylamine to yield the final product. Starting from 6-dimethyl-2,4-dichloroquinazoline, (0.25 g, 1.0 mmol), 25 mg (Yield, 21%) of the final product was isolated; MS (ESI) m/z 434.3.

### Example 35

### Preparation of 4-chloro-N-[4-({[6,8-dimethyl-2-(methylamino)quinazolin-4-yl]amino}methyl)phenyl]-2-fluorobenzamide

4-chloro-N-[4-({[6,8-dimethyl-2-(methylamino)quinazolin-4-yl]amino}methyl)phenyl]-2-fluorobenzamide was prepared starting from 6,8-dimethyl-2,4-dichloroquinazoline, 4-aminobenzylamine and 2-fluoro-4-chlorobenzoylchloride by following the procedure A (step 1). The intermediate product from the (step 1) was aminated using monomethylamine to yield the final product. Starting from 6,8-dimethyl-2,4-dichloroquinazoline, (0.24 g, 1.0 mmol) 40 mg (Yield, 37%) of the final product was isolated. MS (ESI) m/z 464.1.

### Example 36

### Preparation of N-[4-({[2-(dimethylamino)-6-methylquinazolin-4-yl]amino}methyl)phenyl]-3,4-difluorobenzamide

N-[4-({[2-(dimethylamino)-6-methylquinazolin-4-yl]amino}methyl)phenyl]-3,4-difluorobenzamide was prepared starting from 6-methyl-2,4-dichloroquinazoline, 4-aminobenzylamine and 3,4-difluorobenzoylchloride by following the procedure A (step 1). The intermediate product from the (step 1) was aminated using dimethylamine to yield the final product. Starting from 6-methyl-2,4-dichloroquinazoline, (0.24 g, 1.0 mmol), 40 mg (Yield, 31%) of the final product was isolated. MS (ESI) m/z 448.4.

### Example 37

### Preparation of 3,4-dichloro-N-[4-({[6-methyl-2-(methylamino)quinazolin-4-yl]amino}methyl)phenyl]benzamide

3,4-dichloro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 3,4-dichlorobenzoyl chloride by following the procedure A (step 1). The intermediate product from the (step 1) was aminated using monomethylamine to yield the final product. Starting from 6-methyl-2,4-dichloroquinazoline, (0.24 g, 1.0 mmol), 40 mg (Yield, 36%) of the final product was isolated. MS (ESI) m/z 466.3.

### Example 38

### Preparation of N-[4-({[2-(dimethylamino)-6-methylquinazolin-4-yl]amino}methyl)phenyl]-4-fluorobenzamide

N-[4-({[2-(dimethylamino)-6-methylquinazolin-4-yl]amino}methyl)phenyl]-4-fluorobenzamide was prepared starting from 6-methyl-2,4-dichloroquinazoline, 4-aminobenzylamine and 4-fluorobenzoylchloride by following the procedure A (step 1). The intermediate product from the (step 1) was aminated using dimethylamine to yield the final product. Starting from (0.24 g, 1.0 mmol) of 6-dimethyl-2,4-dichloroquinazoline, (0.24 g, 1.0 mmol), 40 mg (Yield, 28%) of the final product was isolated. MS (ESI) m/z 430.3.

### Example 39

### Preparation of N-[4-({[2-(dimethylamino)-6-methylquinazolin-4-yl]amino}methyl)phenyl]-4-fluorobenzamide

A mixture of N-(4-{[(2-chloro-6-methylquinazolin-4-yl)lamino]methyl}phenyl-4-fluorobenzamide) (50 mg, 0.119 mmol), dioxane (3 mL), triethylamine (0.5 mL), dimethylamine (45 mg, 1.0 mmol), was heated to 90° C. during 24 hours. The reaction mixture was cooled at room temperature. The solvent was eliminated and the residue was chromatographed by HPLC to give 22 mg (49% yield) mg of the product.

### Example 40

### Preparation of N-[4-({[2-(dimethylamino)-6-methylquinazolin-4-yl]amino}methylphenyl]-4-fluorobenzamide

N-[4-({[2-(dimethylamino)-6-methylquinazolin-4-yl]amino}methyl)phenyl]-4-fluorobenzamide was prepared starting from 6-methyl-2,4-dichloroquinazoline, 4-aminobenzylamine and 4-fluorobenzoylchloride by following the procedure A (step 1). The intermediate product from the (step 1) was aminated using dimethylamine to yield the final product. Starting from (0.24 g, 1.0 mmol) of 6-dimethyl-2,4-dichloroquinazoline, 270 mg (Yield, 63%) of the final product was isolated. MS (ESI) m/z 430.3.

### Example 41

### Preparation of 3,5-difluoro-N-[4-({[6-methyl-2-(methylamino)quinazolin-4-yl]amino}methyl)phenyl]benzamide

3,5-difluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 3,5-difluorobenzoyl chloride by following the procedure A (step 1). The intermediate product from the (step 1) was aminated using monomethylamine to yield the final product. Starting from (0.24 g, 1.0 mmol) of 6-methyl-2,4-dichloroquinazoline, 30 mg (Yield, 28%) of the final product was isolated. MS (ESI) m/z 434.4.

### Example 42

### Preparation of 4-fluoro-N-(4-{[(6-methyl-2-piperidin-1-ylquinazolin-4-yl)amino]methyl}phenyl)benzamide

4-fluoro-N-(4-{[(6-methyl-2-piperidin-1-ylquinazolin-4-yl)amino]methyl}phenyl)benzamide was prepared starting from 6-methyl-2,4-dichloroquinazoline, 4-aminobenzylamine and 4-fluorobenzoyl chloride by following the procedure A (step 1). The intermediate product from the (step 1) was aminated using piperidine to yield the final product. Starting from (0.24 g, 1.0 mmol) of 6-methyl-2,4-dichloroquinazoline, 30 mg (Yield, 19%) of the final product was isolated. MS (ESI) m/z 470.3.

### Example 43

### Preparation of N-[4-((2-(diethylamino)-6-methylquinazolin-4-ylamino)methyl)phenyl)-4-fluorobenzamide

A mixture of (N-(4-{[(2-chloro-6-methylquinazolin-4-yl)]amino]methyl}phenyl-4-fluorobenzamide) (50 mg, 0.119 mmol), dioxane (3 mL), triethylamine (0.5 mL), diethylamine (44 mg, 0.59 mmol), was heated to 90° C. during 24 hours. The reaction mixture was cooled at room temperature. The solvent was eliminated and the residue was chromatographed by HPLC to give 3.35 (93% yield) mg of the product.

### Example 44

### Preparation of N-(4-((2-(1-azacyclopentyl)-6-methylquinazolin-4-ylamino)methyl)phenyl)-4-fluorobenzamide

A mixture of N-(4-((2-chloro-6-methylquinazolin-4-ylamino)methyl)phenyl)-4-fluorobenzamide (50 mg, 0.119 mmol), dioxane (3 mL), triethylamine (0.5 mL), pyrrolidine (42 mg, 0.59 mmol), was heated to 90° C. during 24 hours. The reaction mixture was cooled at room temperature. The solvent was eliminated and the residue was chromatographed by HPLC to give 1.51 (93% yield) mg of the product.

### Example 45

### Preparation of 4-fluoro-N-(4((6-methyl-2-piperazin-1-yl) quinazolin-4-ylamino)methyl)phenyl)benzamide

A mixture of N-(4-{[(2-chloro-6-methylquinazolin-4-yl)lamino]methyl}phenyl-4-fluorobenzamide) (50 mg, 0.119 mmol), dioxane (3 mL), Triethylamine (0.5 mL), piperazine (51.2 mg, 0.59 mmol), was heated to 90° C. during 24 hours. The reaction mixture was cooled at room temperature. The solvent was eliminated and the residue was chromatographed by HPLC to give 4.5 mg (79% yield) mg of the product.

### Example 46

### Preparation of 4-fluoro-N-((6-methyl-2-(4-methylpiperazin-1-yl)quinazolin-4-ylamino)methyl-phenyl)benzamide

A mixture of (N-(4-{[(2-chloro-6-methylquinazolin-4-yl)lamino]methyl}phenyl-4-fluorobenzamide) (50 mg, 0.57 mmol), dioxane (3 mL), Triethylamine (0.5 mL), 1-methylpiperazine (58 mg, 0.58 mmol), was heated to 90° C. during 24 hours. The reaction mixture was cooled at room temperature. The solvent was eliminated and the residue was chromatographed by HPLC to give 6 mg (90% yield) mg of the product.

### Example 47

### Preparation of 2-fluoro-N-[4-({[6-methyl-2-(methylamino)quinazolin-4-yl]amino}methyl)phenyl]benzamide

2-fluoro-N-[4-({[6-methyl-2-(methylamino)quinazolin-4-yl]amino}methyl)phenyl]benzamide was prepared starting from 6-methyl-2,4-dichloroquinazoline, 4-aminobenzylamine and 2-fluorobenzoyl chloride by following the procedure A (step 1). The intermediate product from the (step 1) was aminated using monomethylamine to yield the final product. Starting from (0.24 g, 1.0 mmol) of 6-methyl-2,4-dichloroquinazoline, 24 mg (Yield, 17%) of the final product was isolated. MS (ESI) m/z 416.3.

### Example 48

### Preparation of 4-fluoro-N-[4-({[6-methyl-2-(4-methylpiperazin-1-yl)quinazolin-4-yl]amino}methyl)phenyl]benzamide

4-fluoro-N-[4-({[6-methyl-2-(4-methylpiperazin-1-yl)quinazolin-4-yl]amino}methyl)phenyl]benzamide was prepared starting from 6-methyl-2,4-dichloroquinazoline, 4-aminobenzylamine and 2-fluorobenzoyl chloride by following the procedure A (step 1). The intermediate product from the (step 1) was aminated using 1-methyl-piperazine to yield the final product. Starting from (0.24 g, 1.0 mmol) of 6-methyl-2,4-dichloroquinazoline, 40 mg (Yield, 15%) of the final product was isolated. MS (ESI) m/z 485.2.

### Example 49

### Preparation of N-[4-({[6,8-dimethyl-2-(4-methylpiperazin-1-yl)quinazolin-4-yl]amino}methyl)phenyl]-4-fluorobenzamide

N-[4-({[6,8-dimethyl-2-(4-methylpiperazin-1-yl)quinazolin-4-yl]amino}methyl)phenyl]-4-fluorobenzamide was prepared starting from 6,8-dimethyl-2,4-dichloroquinazoline, 4-aminobenzylamine and 2-fluorobenzoyl chloride by following the procedure A (step 1). The intermediate product from the (step 1) was aminated using methyl-piperazine to yield the final product. Starting from (0.24 g, 1.0 mmol) of 6-methyl-2,4-dichloroquinazoline, 40 mg (Yield, 23%) of the final product was isolated. MS (ESI) m/z 499.1.

### Example 50

### Preparation of N-{4-[({6,8-dimethyl-2-[(3S)-3-methylpiperazin-1-yl]quinazolin-4-yl}amino)methyl]phenyl}-4-fluorobenzamide

N-{4-[({6,8-dimethyl-2-[(3S)-3-methylpiperazin-1-yl]quinazolin-4-yl}amino)methyl]phenyl}-4-fluorobenzamide was prepared starting from 6,8-dimethyl-2,4-dichloroquinazoline, 4-aminobenzylamine and 2-fluorobenzoyl chloride by following the procedure A (step 1). The intermediate product from the (step 1) was aminated using (S)-2-methylpiperazine to yield the final product. Starting from (0.24 g, 1.0 mmol) of 6,8-methyl-2,4-dichloroquinazoline, 28 mg (Yield, 13%) of the final product was isolated. MS (ESI) m/z 416.3.

### Example 51

### Preparation of 4-fluoro-N-{4-[({6-methyl-2-[(3S)-3-methylpiperazin-1-yl]quinazolin-4-yl}amino)methyl]phenyl}benzamide

4-fluoro-N-{4-[({6-methyl-2-[(3S)-3-methylpiperazin-1-yl]quinazolin-4-yl}amino)methyl]phenyl}benzamide was prepared starting from 6-methyl-2,4-dichloroquinazoline, 4-aminobenzylamine and 2-fluorobenzoyl chloride by following the procedure A (step 1). The intermediate product from the (step 1) was aminated using (S)-2-methylpiperazine to yield the final product. Starting from (0.24 g, 1.0 mmol) of 6-methyl-2,4-dichloroquinazoline, 25 mg (Yield, 29%) of the final product was isolated. MS (ESI) m/z 485.2.

### Example 52

### Preparation of N-[4-({[2-(dimethylamino)-6,8-dimethylquinazolin-4-yl]amino}methyl)phenyl]-4-fluorobenzamide

N-[4-({[2-(dimethylamino)-6,8-dimethylquinazolin-4-yl]amino}methyl)phenyl]-4-fluorobenzamide was prepared starting from 6,8-dimethyl-2,4-dichloroquinazoline, 4-aminobenzylamine and 2-fluorobenzoyl chloride by following the procedure A (step 1). The intermediate product from the (step 1) was aminated using dimethylamine to yield the final product. Starting from (0.24 g, 1.0 mmol) of 6,8-methyl-2,4-dichloroquinazoline, 60 mg (Yield, 44%) of the final product was isolated. MS (ESI) m/z 444.2.

### Example 53

### Preparation of 4-chloro-N-[4-({[2-(dimethylamino)-6-methylquinazolin-4-yl]amino}methyl)phenyl]benzamide

4-chloro-N-[4-({[2-(dimethylamino)-6-methylquinazolin-4-yl]amino}methyl)phenyl]benzamide was prepared starting from 6-methyl-2,4-dichloroquinazoline, 4-aminobenzylamine and 3,5-difluorobenzoyl chloride by following the procedure A (step 1). The intermediate product from the (step 1) was aminated using dimethylamine to yield the final product. Starting from (0.24 g, 1.0 mmol) of 6-methyl-2,4-dichloroquinazoline, 40 mg (Yield, 35%) of the final product was isolated. MS (ESI) m/z 446.1.

### Example 54

### Preparation of Ethyl 4-[4-({4-[(4-fluorobenzoyl)amino]benzyl}amino)-6-methylquinazolin-2-yl]piperazine-1-carboxylate

Ethyl 4-[4-({4-[(4-fluorobenzoyl)amino]benzyl}amino)-6-methylquinazolin-2-yl]piperazine-1-carboxylate was prepared starting from 6-methyl-2,4-dichloroquinazoline, 4-aminobenzylamine and 2-fluorobenzoyl chloride by following the procedure A (step 1). The intermediate product from the (step 1) was aminated using piperazine-1-carboxylic ethyl ester to yield the final product. Starting from (0.24 g, 1.0 mmol) of 6-methyl-2,4-dichloroquinazoline, 500 mg (Yield, 65%) of the final product was isolated. MS (ESI) m/z 543.3.

### Example 55

### Preparation of 4-fluoro-N-[4-({[6-methyl-2-(4-pyridin-2-ylpiperazin-1-yl)quinazolin-4-yl]amino}methyl)phenyl]benzamide

4-fluoro-N-[4-({[6-methyl-2-(4-pyridin-2-ylpiperazin-1-yl)quinazolin-4-yl]amino}methyl)phenyl]benzamide was prepared starting from 6-methyl-2,4-dichloroquinazoline, 4-aminobenzylamine and 2-fluorobenzoyl chloride by following the procedure A (step 1). The intermediate product from the (step 1) was aminated using 1-pyridin-2-piperazine to yield the final product. Starting from (0.24 g, 1.0 mmol) of 6,8-methyl-2,4-dichloroquinazoline, 140 mg (Yield, 44%) of the final product was isolated. MS (ESI) m/z 548.3.

### Example 56

### Preparation of N-(4-{[(2-azepan-1-yl-6-methylquinazolin-4-yl)amino]methyl}phenyl)-4-fluorobenzamide

N-(4-{[(2-azepan-1-yl-6-methylquinazolin-4-yl)amino]methyl}phenyl)-4-fluorobenzamide was prepared starting from 6-methyl-2,4-dichloroquinazoline, 4-aminobenzylamine and 2-fluorobenzoyl chloride by following the procedure A (step 1). The intermediate product from the (step 1) was aminated using homopiperidine to yield the final product. Starting from (0.24 g, 1.0 mmol) of 6-methyl-2,4-dichloroquinazoline, 60 mg (Yield, 20%) of the final product was isolated. MS (ESI) m/z 484.3.

### Example 57

### Preparation of N-[4-({[2-(4-ethylpiperazin-1-yl-6-methylquinazolin-4-yl]amino}methyl)phenyl]-4-fluorobenzamide

N-[4-({[2-(4-ethylpiperazin-1-yl)-6-methylquinazolin-4-yl]amino}methyl)phenyl]-4-fluorobenzamide was prepared starting from 6-methyl-2,4-dichloroquinazoline, 4-aminobenzylamine and 2-fluorobenzoyl chloride by following the procedure A (step 1). The intermediate product from the (step 1) was aminated using 4-ethyl piperazine to yield the final product. Starting from (0.24 g, 1.0 mol) of 6-methyl-2,4-dichloroquinazoline, 480 mg (Yield, 74% of the final product was isolated. MS (ESI) m/z 499.3; MS (ESI) m/z 250.1.

### Example 58

### Preparation of 4-fluoro-N-{4-[({6-methyl-2-[methyl(pyridin-2-ylmethyl)amino]quinazolin-4-yl}amino)methyl]phenyl}benzamide

4-fluoro-N-{4-[({6-methyl-2-[methyl(pyridin-2-ylmethyl)amino]quinazolin-4-yl}amino)methyl]phenyl}benzamide was prepared starting from 6-methyl-2,4-dichloroquinazoline, 4-aminobenzylamine and 4-fluorobenzoyl-chloride by following the procedure A (step 1). The intermediate product from the (step 1) was aminated using benzyl-methyl-amine to yield the final product. Starting from (0.24 g, 1.0 mmol) of 6-methyl-2,4-dichloroquinazoline, 50 mg (Yield, 18%) of the final product was isolated. MS (ESI) m/z 507.3.

### Example 59

### Preparation of N-{4-[({2-(dimethylamino)-6-[6-(dimethylamino)pyridin-3-yl]quinazolin-4-yl}amino)methyl]phenyl}-4-fluorobenzamide

N-{4-[({2-(dimethylamino)-6-[6-(dimethylamino)pyridin-3-yl]quinazolin-4-yl}amino)methyl]phenyl}-4-fluorobenzamide was prepared starting from 6-Iodo-2,4-dichloroquinazoline, 4-aminobenzylamine and 4-fluorobenzoyl-chloride by following the procedure A (step 1). The intermediate product from the (step 1) was aminated using dimethylamine followed by Suzuki coupling with 4-dimethylamine-pyridine boronic acid to yield the final product. Starting from (0.24 g, 1.0 mmol) of 6-iodo-2,4-dichloroquinazoline, 112 mg (Yield, 45%); MS (ESI) m/z 536.1.

### Example 60

### Preparation of N-[4-({[2-(dimethylamino)-6-fluoroquinazolin-4-yl]amino}methyl)phenyl]-4-fluorobenzamide

N-[4-({[2-(dimethylamino)-6-fluoroquinazolin-4-yl]amino}methyl)phenyl]-4-fluorobenzamide was prepared starting from 6-fluoro-2,4-dichloroquinazoline, 4-aminobenzylamine and 4-fluorobenzoyl-chloride by following the procedure A (step 1). The intermediate product from the (step 1) was aminated using dimethylamine to yield the final product. Starting from (0.24 g, 1.0 mmol) of 6-fluoro-2,4-dichloroquinazoline, 200 mg (Yield, 67%) of the final product was isolated. MS (ESI) m/z 433.

### Example 61

### Preparation of N-[4-({[2-(dimethylamino)-7-isopropyylquinazolin-4-yl]amino}methyl)phenyl]-4-fluorobenzamide

N-[4-({[2-(dimethylamino)-7-isopropylquinazolin-4-yl]amino}methyl)phenyl]-4-fluorobenzamide was prepared starting from 7-iso-propyl-2,4-dichloroquinazoline, 4-aminobenzylamine and 4-fluorobenzoyl chloride by following the procedure A (step 1). The intermediate product from the (step 1) was aminated using dimethylamine to yield the final product. Starting from (0.22 g, 0.92 mmol) of 7-iso-propyl-2,4-dichloroquinazoline, 160 mg Yield, 72%) of the final product was isolated. MS (ES) m/z 458.4.

### Example 62

### Preparation of 6-chloro-N-[4-({[2-(dimethylamino)-7-isopropylquinazolin-4-yl]amino}methyl)phenyl]nicotinamide

6-chloro-N-[4-({[2-(dimethylamino)-7-isopropylquinazolin-4-yl]amino}methyl)phenyl]nicotinamide was prepared starting from 7-iso-propyl-2,4-dichloroquinazoline, 4-aminobenzylamine and 6-nicotinoyl chloride by following the procedure A (step 1). The intermediate product from the (step 1) was aminated using dimethylamine to yield the final product. Starting from (0.22 g, 0.92 mmol) of 7-iso-propyl-2,4-dichloroquinazoline, 80 mg (Yield, 40%) of the final product was isolated. MS (ESI) m/z 475.3.

### Example 63

### Preparation of 1-benzyl-N-[4-({[2-(dimethylamino)-7-isopropylquinazolin-4-yl]amino}methyl)phenyl]piperidine-4-carboxamide

1-Benzyl-N-[4-({[2-(dimethylamino)-7-isopropylquinazolin-4-yl]amino}methyl)phenyl]piperidine-4-carboxamide was prepared starting from 7-iso-propyl-2,4-dichloroquinazoline, 4-aminobenzylamine and 1-benzyl-piperidine-4-carboxylic acid (4-aminomethyl-phenyl)-amide by following the procedure A (step 1). The intermediate product from the (step 1) was aminated using dimethylamine to yield the final product. Starting from (0.22 g, 0.92 mmol) of 7-iso-propyl-2,4-dichloroquinazoline, 35 mg (Yield, 12%) of the final product was isolated. MS (ESI) m/z 537.1.

### Example 64

### Preparation of 6-chloro-N-[4-({[2-(dimethylamino)-7-fluoro-8-methylquinazolin-4-yl]amino}methyl)phenyl]nicotinamide

6-chloro-N-[4-({[2-(dimethylamino)-7-fluoro-8-methylquinazolin-4-yl]amino}methyl)phenyl]nicotinamide was prepared starting from 7-fluoro-8-methyl-propyl-2,4-dichloroquinazoline, 4-aminobenzylamine and 6-nicotinoyl chloride amide by following the procedure A (step 1). The intermediate product from the (step 1) was aminated using dimethylamine to yield the final product. Starting from (0.4 g, 0.88 mmol) of 7-fluoro-8-methyl-propyl-2,4-dichloroquinazoline, 100 mg (Yield, 38%) of the final product was isolated. MS (ESI) m/z 465.3.

### Example 65

### Preparation of 6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide

6-chloro-N-[4-({[8-methyl-2-(methylamino)quinazolin-4-yl]amino}methyl)phenyl]nicotinamide was prepared starting from 8-methyl-2,4-dichloroquinazoline, 4-aminobenzylamine and 6-chloronicotinoylchloride by following the procedure A (step 1). The intermediate product from the step 1 was aminated using monomethylamine to yield the final product. Starting from of 8-methyl-2,4-dichloroquinazoline, (213 mg, 0.1 mmol), 250 mg (Yield, 23%) of the final product was isolated. MS (ESI) m/z 433.2. During this reaction 14% of 6-(methylamino)-N-[4-({[8-methyl-2-(methylamino)quinazolin-4-yl]amino}methyl)phenyl]nicotinamide was also isolated. MS (ESI) m/z 428.3.

### Example 66

### Preparation of N-[4-({[6-bromo-8-methyl-2-(methylamino)quinazolin-4-yl]amino}methyl)phenyl]-4-chlorobenzamide

Step 1: To a stirred solution of 6-bromo-2,4-dichloro-8-methylquinazoline, (290 mg, 1 mmol) in CH₂Cl₂ N-[4-(aminomethyl)phenyl]-4-chlorobenzamide (259 mg, 0.1 mmol) was added in the presence of triethylamine (5 mL) at room temperature. The reaction mixture was stirred for 8 h and quenched with ice cold water. It was extracted with chloroform and washed well with water. Organic layer was dried and concentrated. The resultant product N-(4-{[(6-bromo-2-chloro-8-methylquinazolin-4-yl)amino]methyl}phenyl)-4-chlorobenzamide was purified by silica gel column chromatography by eluting with 1:1 ethylacetate; hexane. Yield, 260 mg, 51%; MS (ESI) m/z 516.2.

Step 2: A mixture of N-(4-{[(6-bromo-2-chloro-8-methylquinazolin-4-yl)amino]methyl}phenyl)-4-chlorobenzamide (600 mg, 1.2 mmol) and monomethylamine (2M. solution in THF) was heated in a sealed tube at 100° C. for 24 h. At the end, reaction mixture was concentrated and extracted with 3:1 (CHCl₃: MeOH). Organic layer was washed once with water and dried over anhydrous MgSO₄. It was filtered and concentrated. The solid obtained was suspended in ethylacetate and filtered. The product obtained was found to be pure enough for further transformations. Yield, 450 mg, 68%; MS (ESI) m/z 510.1 mp 139° C.

### Example 67

### Preparation of 4-chloro-N-[4-({[6-(2-furyl)-8-methyl-2-(methylamino)quinazolin-4-yl]amino}methyl)phenyl]benzamide

A mixture of N-[4-({[6-bromo-8-methyl-2-(methylamino)quinazolin-4-yl]amino}methyl)phenyl]-4-chlorobenzamide (150 mg, 0.29 mmol) and the 2-(tributylstannyl)furan (357 mg, 1 mmol) and tetrakis(triphenylphosphine)palladium (0) (50 mg) was refluxed in degassed toluene (100 mL) for 48 hrs. At the end, reaction mixture was filtered through a pad of Diatomaceous earth and concentrated. The crude product was purified by silica-gel column chromatography by eluting it with 5% methanol: ethylacetate. Brown solid, Yield, 50 mg (34%), MS (ESI) m/z 498.1.

### Example 68

### Preparation of N-[4-({[2-(dimethylamino)-6-iodoquinazolin-4-yl]amino}methyl)phenyl]-4-fluorobenzamide

Starting from the 6-iodo-2,4-dichloroquinazoline (975 mg, 3.0 mmol) and N-[4-(aminomethyl)phenyl]-4-fluorobenzamide (732 mg, 3.0 mmol) and following the procedure outlined for the example 66, step 1, N-(4-{[(2-chloro-6-iodoquinazolin-4-yl)amino]methyl}phenyl)-4-fluorobenzamide was isolated as an amorphous solid. Yield, 900 mg, 56%; MS (ESI) m/z 533.2.

Starting from N-(4-{[(2-chloro-6-iodoquinazolin-4-yl)amino]methyl}phenyl)-4-fluorobenzamide (900 mg, 1.7 mmol), N,N-dimethyl amine (40% solution in THF) and following the procedure outlined for the example 66, step 2, N-[4-({[2-(dimethylamino)-6-iodoquinazolin-4-yl]amino}methyl)phenyl]-4-fluorobenzamide was isolated as an yellow solid. Yield, 800 mg, 87%: MS (ESI) m/z 542.2.

### Example 69

### Preparation of N-{4-[({2-(dimethylamino)-6-[3-(dimethylamino)prop-1-yn-1-yl]quinazolin-4-yl}amino)methyl]phenyl}-4-fluorobenzamide

A mixture of N-[4-({[2-(dimethylamino)-6-iodoquinazolin-4-yl]amino}methyl)phenyl]-4-fluorobenzamide (100 mg, 0.18 mmol), Dichlorobis(triphenylphosphine)palladium(II) (PdCl₂(PPh₃)₂) (60 mg), copper iodide (CuI) (100 mg), 3-dimethylamino-1-propyne (100 mg, excess) and triethylamine (4 ml) was refluxed together in acetonitrile for 12 h. At the end, reaction mixture was filtered through a pad of diatomaceous earth and concentrated. The residue was dissolved in chloform; methanol (3:1) and washed once with water. The crude product was purified by silica-gel column chromatography by eluting it with 10% methanol/ethylacetate. Yield, 64 mg, 72%; MS (ESI) m/z 497.3.

### Example 70

### Preparation of methyl 2-(dimethylamino)-4-({4-[(4-fluorobenzoyl)amino]benzyl}amino)quinazoline-6-carboxylate

To a stirred refluxing solution of N-[4-({[2-(dimethylamino)-6-iodoquinazolin-4-yl]amino}methyl)phenyl]-4-fluorobenzamide (100 mg, 0.18 mmol), PdCl₂(PPh₃)₂ (60 mg) and triethylamine (5 mL) in methanol (50 mL), carbon monoxide (CO) was passed for 48 hrs in a balloon. At the end, reaction mixture was filtered through a pad of celite and concentrated. The residue was purified through a silica-gel column chromatography by eluting it with 10% methanol/ethylacetate. Yield, 40 mg, 46%; MS (ESI) m/z 474.1.

### Example 71

### Preparation of N-[4-({[2-(dimethylamino)-6-(hydroxymethyl)quinazolin-4-yl]amino}methyl)phenyl]-4-fluorobenzamide

To a stirred suspension of LiAlH₄ (60 mg) in THF at 0° C., a THF solution of 1methyl 2-(dimethylamino)-4-({4-[(4-fluorobenzoyl)amino]benzyl}amino)quinazoline-6-carboxylate (200 mg, 0.42 mmol) was slowly added. After the addition, reaction mixture was stirred for 1 h and quenched with ice cold water. The product was extracted with chloroform and washed well water. It was dried over anhydrous MgSO₄; filtered and concentrated. Product was purified by silica-gel column chromatography by eluting it with 10% methanol/ethylacetate along with 5 mL/lit 30% NH₄OH. Yield, 80 mg, 43%; MS (ESI) m/z 446.3.

### Example 72

### Preparation of 6-chloro-N-[4-({[2-(dimethylamino)-6-iodoquinazolin-4-yl]amino}methyl)phenyl]nicotinamide

Starting from the 6-iodo-2,4-dichloroquinazoline (1625 mg, 5 mmol) and N-[4-(aminomethyl)phenyl]-6-chloronicotinamide (1300 mg, 5.0 mmol) and following the procedure outlined for the example 66, step 1, 6-chloro-N-(4-{[(2-chloro-6-iodoquinazolin-4-yl)amino]methyl}phenyl)nicotinamide was isolated as an amorphous solid. Yield, 1.5 g, 54%; MS (ESI) m/z 550.1.

Starting from 6-chloro-N-(4-{[(2-chloro-6-iodoquinazolin-4-yl)amino]methyl}phenyl)nicotinamide (2000 mg, 3.6 mmol), N,N-dimethylamine (40% solution in THF) and following the procedure outlined for the example 66, step 2, 6-chloro-N-[4-({[2-(dimethylamino)-6-iodoquinazolin-4-yl]amino}methyl)phenyl]nicotinamide was isolated as an yellow solid. Yield, 2000 mg, 98%: MS (ESI) m/z 559.1.

### Example 73

### Preparation of 6-chloro-N-[4-({[2-(dimethylamino)-6-vinylquinazolin-4-yl]amino}methyl)phenyl]nicotinamide

A mixture of 6-chloro-N-[4-({[2-(dimethylamino)-6-iodoquinazolin-4-yl]amino}methyl)phenyl]nicotinamide (300 mg, 0.53 mmol), n-tributylvinyl tin (500 mg, excess) and tetrakis(triphenylphosphine)palladium (0) (50 mg) was heated in Dimethylformamide (DMF) at 100° C. for 8 h. After the completion, reaction mixture was filtered through a pad of diatomaceous earth and concentrated. The residue obtained was extracted with chloroform and washed well with water. Organic layer was dried over anhydrous MgSO₄; filtered and concentrated. The crude product was purified by silica-gel column chromatography. Initially the column was eluted with ethylacetate and latter with 20% methanol/ethylacetate and 1.5% aqueous ammonium hydroxide (NH₄OH) solution. Yield 190 mg, 78%; MS (ESI) m/z 459.2.

### Example 74

### Preparation of 1-benzyl-N-[4-({[2-(dimethylamino)-6-iodoquinazolin-4-yl]amino}methyl)phenyl]piperidine-4-carboxamide

Starting from the 6-iodo-2,4-dichloroquinazoline (650 mg, 2 mmol) and N-[4-(aminomethyl)phenyl]-1-benzylpiperidine-4-carboxamide (646 mg, 2.0 mmol) and following the procedure outlined for the example 66, step 1, 1-benzyl-N-(4-{[(2-chloro-6-iodoquinazolin-4-yl)amino]methyl}phenyl)piperidine-4-carboxamide was isolated as an amorphous solid. Yield, 700 mg, 57%; MS (ESI) m/z 611.9.

Starting from 1-benzyl-N-(4-{[(2-chloro-6-iodoquinazolin-4-yl)amino]methyl}phenyl)piperidine-4-carboxamide (620 mg, 1.0 mmol), N,N-dimethylamine (40% solution in THF) and following the procedure outlined for the example 66, step 2, 1-benzyl-N-[4-({[2-(dimethylamino)-6-iodoquinazolin-4-yl]amino}methyl)phenyl]piperidine-4-carboxamide was isolated as an yellow solid. Yield, 600 mg, 96%: MS (ESI) m/z 621.5.

### Example 75

### Preparation of 1-benzyl-N-[4-({[2-(dimethylamino)-6-vinylquinazolin-4-yl]amino}methyl)phenyl]-piperidine-4-carboxamide

A mixture of 1-benzyl-N-[4-({[2-(dimethylamino)-6-iodoquinazolin-4-yl]amino}methyl)phenyl]piperidine-4-carboxamide (220 mg, 0.35 mmol), n-tributylvinyl tin (200 mg, excess) and tetrakis(triphenylphosphine)palladium (0) (30 mg) was heated in DMF (25 mL) at 100° C. for 8 h. After the completion, reaction mixture was filtered through a pad of diatomaceous earth and concentrated. The residue obtained was extracted with chloroform and washed well with water. Organic layer was dried over anhydrous MgSO₄; filtered and concentrated. The crude product was purified by silica-gel column chromatography. Initially the column was eluted with ethylacetate and latter with 20% methanol/ethylacetate and 1.5% aqueous NH₄OH solution. Yield 43 mg, 25%; MS (ESI) m/z 521.5.

### Example 76

### Preparation of 1-benzyl-N-[4-({[2-(dimethylamino)-8-methylquinazolin-4-yl]amino}methyl)phenyl]piperidine-4-carboxamide

Starting from the 8-methyl-2,4-dichloroquinazoline (100 mg, 0.47 mmol) and N-[4-(aminomethyl)phenyl]-1-benzylpiperidine-4-carboxamide (200 mg, excess) and following the procedure outlined for the example 66, step 1, 1-benzyl-N-(4-{[(2-chloro-8-methylquinazolin-4-yl)amino]methyl}phenyl)piperidine-4-carboxamide was isolated as an amorphous solid. Yield, 100 mg, 42%; MS (ESI) m/z 500.3.

Starting from 1-benzyl-N-(4-{[(2-chloro-8-methylquinazolin-4-yl)amino]methyl}phenyl)piperidine-4-carboxamide (100 mg, 0.2 mmol), N,N-dimethylamine (40% solution in THF) and following the procedure outlined for the example 66, step 2, 1-benzyl-N-[4-({[2-(dimethylamino)-8-methylquinazolin-4-yl]amino}methyl)phenyl]piperidine-4-carboxamide was isolated as an yellow solid. Yield, 25 mg, 25%: MS (ESI) m/z 509.6.

### Example 77

### Preparation of 1-benzyl-N-(4-{[(6-methyl-2-pyrrolidin-1-ylquinazolin-4-yl)amino]methyl}phenyl)piperidine-4-carboxamide

Starting from the 6-methyl-2,4-dichloroquinazoline (426 mg, 2.0 mmol) and N-[4-(aminomethyl)phenyl]-1-benzylpiperidine-4-carboxamide (646 mg, 2.0 mmol) and following the procedure outlined for the example 66, step 1, 1-benzyl-N-(4-{[(2-chloro-6-methylquinazolin-4-yl)amino]methyl}phenyl)piperidine-4-carboxamide was isolated as a white solid. Yield, 500 mg, 50%; MS (ESI) m/z 500.3.

Starting from 1-benzyl-N-(4-{[(2-chloro-6-methylquinazolin-4-yl)amino]methyl}phenyl)piperidine-4-carboxamide (30 mg, 0.06 mmol), pyrrolidine (15 mg, excess) and following the procedure outlined for the example 66, step 2, 1-benzyl-N-(4-{[(6-methyl-2-pyrrolidin-1-ylquinazolin-4-yl)amino]methyl}phenyl)piperidine-4-carboxamide was isolated. The product was purified by High performance liquid chromatography (HPLC) using acetonitrile/water/NH₄OH-gradient as eluent. MS (ESI) m/2 268.1.

### Example 78

### Preparation of 1-benzyl-N-{4-[({2-[(3R,5S)-3,5-dimethylpiperazin-1-yl]-6-methylquinazolin-4-yl}amino)methyl]phenyl}piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), 2,6-dimethylpiperazine (20 mg, excess) and following the procedure outlined for the example 66, step 2, 1-benzyl-N-{4-[({2-[(3R,5S)-3,5-dimethylpiperazin-1-yl]-6-methylquinazolin-4-yl}amino)methyl]phenyl}piperidine-4-carboxamide was isolated. The product was purified by HPLC using acetonitrile/water/NH₄OH-gradient as eluent. MS (ESI) m/z 578.

### Example 79

### Preparation of 1-benzyl-N-[4-({[6-methyl-2-(4-pyridin-2-ylpiperazin-1-yl)quinazolin-4-yl]amino}methyl)phenyl]piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), 1-(2-pyridyl)piperazine (20 mg, excess) and following the procedure outlined for the example 66, step 2, 1-benzyl-N-[4-({[6-methyl-2-(4-pyridin-2-ylpiperazin-1-yl)quinazolin-4-yl]amino}methyl)phenyl]piperidine-4-carboxamide was isolated. The product was purified by HPLC using acetonitrile/water/NH₄OH-gradient as eluent. MS (ESI) m/z 627.

### Example 80

### Preparation of 1-benzyl-N-[4-({[2-(2,5-dihydro-1H-pyrrol-1-yl)-6-methylquinazolin-4-yl]amino}methyl)phenyl]piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), pyrroline (20 mg, excess) and following the procedure outlined for the example 66, step 2, 1-benzyl-N-[4-({[2-(2,5-dihydro-1H-pyrrol-1-yl)-6-methylquinazolin-4-yl]amino}methyl)phenyl]piperidine-4-carboxamide was isolated. The product was purified by HPLC using acetonitrile/water/NH₄OH-gradient as eluent. MS (ESI) m/z 533.

### Example 81

### Preparation of 1-benzyl-N-{4-[({2-[(2-furylmethyl)amino]-6-methylquinazolin-4-yl}amino)methyl]phenyl}piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), furylamine (20 mg, excess) and following the procedure outlined for the example 66, step 2, 1-benzyl-N-{4-[({2-[(2-furylmethyl)amino]-6-methylquinazolin-4-yl}amino)methyl]phenyl}piperidine-4-carboxamide was isolated. The product was purified by HPLC using acetonitrile/water/NH₄OH-gradient as eluent. MS (ESI) m/z 561.

### Example 82

### Preparation of 1-benzyl-N-[4-({[6-methyl-2-(4-pyrimidin-2-ylpiperazin-1-yl)quinazolin-4-yl]amino}methyl)phenyl]piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), 1-(2-pyrimidyl)piperazine dihydrochloride (30 mg, excess) and following the procedure outlined for the example 66, step 2, 1-benzyl-N-[4-({[6-methyl-2-(4-pyrimidin-2-ylpiperazin-1-yl)quinazolin-4-yl]amino}methyl)phenyl]piperidine-4-carboxamide was isolated. The product was purified by HPLC using acetonitrile/water/NH₄OH-gradient as eluent. MS (ESI) m/z 628.

### Example 83

### Preparation of 1-benzyl-N-[4-({[6-methyl-2-(4-pyrrolidin-1-ylpiperidin-1-yl)quinazolin-4-yl]amino}methyl)phenyl]piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), 4-(1-pyrrolidinyl)piperidine (30 mg, excess) and following the procedure outlined for the example 66, step 2, 1-benzyl-N-[4-({[6-methyl-2-(4-pyrrolidin-1-ylpiperidin-1-yl)quinazolin-4-yl]amino}methyl)phenyl]piperidine-4-carboxamide was isolated. The product was purified by HPLC using acetonitrile/water/NH₄OH-gradient as eluent. MS (ESI) m/z 618.

### Example 84

### Preparation of N-(4-{[(2-azetidin-1-yl-6-methylquinazolin-4-yl)amino]methyl}phenyl)-1-benzylpiperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), azetidine hydrochloride (30 mg, excess) and following the procedure outlined for the example 66, step 2, N-(4-{[(2-azetidin-1-yl-6-methylquinazolin-4-yl)amino]methyl}phenyl)-1-benzylpiperidine-4-carboxamide was isolated. The product was purified by HPLC using acetonitrile/water/NH₄OH-gradient as eluent. MS (ESI) m/z 521.

### Example 85

### Preparation of 1-benzyl-N-{4-[({6-methyl-2-[(3R)-3-methylpiperazin-1-yl]quinazolin-4-yl}amino)methyl]phenyl}piperidine-4-carboxamide

Starting from 1-benzyl-N-(4-{[(2-chloro-6-methylquinazolin-4-yl)amino]methyl}phenyl)piperidine-4-carboxamide (30 mg, 0.06 mmol), 2-methylpiperazine (30 mg, excess) and following the procedure outlined for the example 66, step 2, 1-benzyl-N-{4-[({6-methyl-2-[(3R)-3-methylpiperazin-1-yl]quinazolin-4-yl}amino)methyl]phenyl}piperidine-4-carboxamide was isolated. The product was purified by HPLC using acetonitrile/water/NH₄OH-gradient as eluent. MS (ESI) m/z 564.

### Example 86

### Synthesis of N-[4-({[2-(dimethylamino)-7-iodoquinazolin-4-yl]amino}methyl)phenyl]-4-fluorobenzamide

### Step 1: Synthesis of ethyl {[(3-iodophenyl)amino]carbonothioyl}carbamate

To a solution of 3-iodoaniline (11.4 g, 52 mmol) in CH₂Cl₂ (150 mL) was added a solution of ethyl isothiocyanatoformate (6.14 mL, 52 mmol) in CH₂Cl₂ (20 mL). The mixture was stirred at RT for 2 h, and concentrated under reduced pressure to give off-white solid (18.2 g, 100% yield). MS (ESI) m/z 351.1.

### Step 2: Synthesis of 2-(ethylthio)-7-iodoquinazolin-4(3H)-one

To a solution of ethyl {[(3-iodophenyl)amino]carbonothioyl}carbamate (18.2 g, 52 mmol) in acetone (200 mL) was added dropwise of EtI (4.16 mL, 52 mmol) and K₂CO₃ (21.5 g, 156 mmol). The resulting mixture was stirred at RT overnight, and filtered through a pad of Diatomaceous earth. The filtration was concentrated in vacuum, and the residue was dissolved in CH₂Cl₂ (200 mL). The solution was washed with water and brine, dried over (MgSO₄). Evaporation of solvent gave yellow oil (19.0 g), which was dissolved in phenyl ether (200 mL). The mixture was heated at 200° C. overnight, and cooled to RT, during which time, a lot of white precipitate was formed. The mixture was diluted with hexanes, and filtered to give the title compound as off-white solid (10.8 g, 63% yield). MS (ESI) m/z 333.1.

### Step 3: Synthesis of 7-iodoquinazoline-2,4(1H,3H)-dione

To a solution of 2-(ethylthio)-7-iodoquinazolin-4(3H)-one (10.8 g, 32.5 mmol) in ethanol (40 mL) was added 6N HCl (40 mL). The mixture was heated at 80° C. overnight, then cooled down to RT, during which time, a lot of white precipitate was formed. The resulting solid was collected by filtration to give off-white solid (8.97 g, 96% yield). MS (ESI) m/z 289.0.

### Step 4: Synthesis of 2,4-dichloro-7-iodoquinazoline

POCl₃ (10 mL) was added to 7-iodoquinazoline-2,4(1H,3H)-dione (3.78 g, 13.1 mmol), followed by addition of N,N-dimethylaniline (1 mL). The resulting mixture was heated at 115° C. for 6 h. After cooling to RT, Most of POCl₃ was removed by distillation under reduced pressure. The residue was poured into ice-water, ammonium hydroxide was added to adjust pH to 5-7. The mixture was extracted several times with CH₂Cl₂, and the combined extracts were washed with brine, and dried over (MgSO₄). The solvent was removed under reduced pressure, and the residue was purified by flash chromatography (Ethyl acetate (EtOAc):CH₂Cl₂=5:95) to give 2,4-dichloro-7-iodoquinazoline as off-white solid (3.56 g, 84%); Melting point (Mp): 163° C.; MS (ESI) m/z 324.9.

### Step 5: Synthesis of N-(4-{[(2-chloro-7-iodoquinazolin-4-yl)amino]methyl}phenyl)-4-fluorobenzamide

To a solution of N-[4-(aminomethyl)phenyl]-4-fluorobenzamide (537 mg, 2.2 mmol) in CH₂Cl₂ (10 mL) was added Et₃N (0.84 mL, 6.0 mmol), followed by addition of 2,4-dichloro-7-iodoquinazoline (648 mg, 2.0 mmol). The resulting mixture was stirred at RT for 20 h, during which time a lot of precipitate was formed. The resulting solid was collected by filtration and washed with small amount of EtOAc and water to give the expected product as off-white solid (745 mg, yield: 70%). MS (ESI) m/z 533.0.

### Step 6: Synthesis of N-[4-({[2-(dimethylamino)-7-iodoquinazolin-4-yl]amino}methyl)phenyl]-4-fluorobenzamide

To a solution of N-(4-{[(2-chloro-7-iodoquinazolin-4-yl)amino]methyl}phenyl)-4-fluorobenzamide (532 mg, 1 mmol) in THF (2 mL) was added dimethylamine solution (40% in water, 0.6 mL, 5 mmol). The resulting mixture was heated at 100° C. in a sealed tube for 26 h, then cooled to RT. The solvent was evaporated under reduced pressure, and the residue was purified by flash chromatography (CH₂Cl₂:CH₃OH=90:10) to give off-white solid (487 mg, yield: 90%). MS (ESI)m/z 542.1.

### Example 87

### Synthesis of N-[4-({[2-(dimethylamino)-7-vinylquinazolin-4-yl]amino}methyl)phenyl]-4-fluorobenzamide

To a solution of N-[4-({[2-(dimethylamino)-7-iodoquinazolin-4-yl]amino}methyl)phenyl]-4-fluorobenzamide (541 mg, 1 mmol) in DMF (5 mL), was added PdCl₂(PPh₃)₂ (35 mg, 5 mol %) as catalyst, followed by addition of tributyl(vinyl)tin (0.35 mL, 1.2 mmol). The resulting mixture was heated at 90° C. under nitrogen for 3 h. Upon completion, the reaction mixture was cooled down to RT, and then poured into cold water. The resulting solid was collected by filtration, and then purified by flash chromatography (CH₂Cl₂:CH₃OH=90:10) to give off-white solid, which was treated with HCl in methanol to form HCl salt as off-white solid (292 mg, yield: 61%). Mp: 145° C.; MS (ESI) m/z 442.1.

### Example 88

### Preparation of N-[4-({[2-(dimethylamino)-7-ethylquinazolin-4-yl]amino}methyl)phenyl]-4-fluorobenzamide

To a solution of N-[4-({[2-(dimethylamino)-7-vinylquinazolin-4-yl]amino}methyl)phenyl]-4-fluorobenzamide (239 mg, 0.5 mmol) in MeOH (10 mL) was added Pd/C (10%) catalyst (48 mg). The resulting mixture was hydrogenated at RT for 2 h. The reaction mixture was filtered through a pad of Diatomaceous earth, washed with methanol, and the resulting filtrate was concentrated under reduced pressure. The residue was subjected to HPLC separation to give off-white solid, which was converted to HCl salt as off-white solid (229 mg, yield: 96%). Mp: 270° C.; MS (ESI) m/z 444.2.

### Example 89

### Preparation of N-[4-({[7-cyano-2-(dimethylamino)quinazolin-4-yl]amino}methyl)phenyl]-4-fluorobenzamide

To a solution of N-[4-({[2-(dimethylamino)-7-iodoquinazolin-4-yl]amino}methyl)phenyl]-4-fluorobenzamide (252 mg, 0.46 mmol) in DMF 9 (mL) were added zinc cyanide (Zn(CN)₂) (66 mg, 0.56 mmol) and tetrakis(triphenylphosphine)palladium(0) (Pd(PPh₃)₄) (26 mg, 5 mol %). The resulting mixture was heated at 80° C. under N₂ for 5 h. Upon completion, the reaction mixture was cooled to RT, and poured into cold water. The resulting solid was collected by filtration, and purified by HPLC, and then converted to HCl salt to give off-white solid (156 mg, 70%). Mp: 150° C.; MS (ESI) m/z 441.1.

### Example 90

### Preparation of N-[4-({[7-(aminomethyl)-2-(dimethylamino)quinazolin-4-yl]amino}methyl)phenyl]-4-fluorobenzamide

To a solution of N-[4-({[7-cyano-2-(dimethylamino)quinazolin-4-yl]amino}methyl)phenyl]-4-fluorobenzamide (142 mg, 0.32 mmol) in MeOH (10 mL) was added Pd/C (10%) catalyst (28 mg) and concentrated (Conc.) HCl (30%, 0.2 mL). The resulting mixture was hydrogenated at RT for 48 h. The reaction mixture was filtered through a pad of Diatomaceous earth, washed with methanol, and the resulting filtrate was concentrated under reduced pressure to give off-white solid (HCl salt, 140 mg, yield: 96%). Mp: 245° C.; MS (ESI) m/z 445.4.

### Example 91

### Preparation of N-{4-[({2-(dimethylamino)-7-[(dimethylamino)methyl]quinazolin-4-yl}amino)methyl]phenyl}-4-fluorobenzamide

To a solution of N-[4-({[7-(aminomethyl)-2-(dimethylamino)quinazolin-4-yl]amino}methyl)phenyl]-4-fluorobenzamide HCl salt (110 mg, 0.21 mmol) in MeOH (2 mL) were added formaldehyde (37%, 68 μL, 0.84 mmol), NaBH₃CN (13 mg, 0.21 mmol) and ZnCl₂ (14 mg, 0.10 mmol). The resulting mixture was stirred at RT overnight. The mixture was filtered, and washed with methanol. The resulting filtrate was concentrated in vacuum, and subjected to HPLC separation, and then converted the product to HCl salt to give off-white solid (68 mg, 60%); Mp: 94° C.; MS (ESI) m/z 473.3.

### Example 92

### Preparation of N-[4-({[2-(dimethylamino)-7-formylquinazolin-4-yl]amino}methyl)phenyl]-4-fluorobenzamide

To a solution of N-[4-({[2-(dimethylamino)-7-iodoquinazolin-4-yl]amino}methyl)phenyl]-4-fluorobenzamide (417 mg, 0.75 mmol) in DMF 3 (mL) were added sodium formate (HCO₂Na) (102 mg, 1.5 mmol) and PdCl₂(PPh₃)₂ (26 mg, 5 mol %). The resulting mixture was heated at 100° C. under CO (gas, 1 atm) for 5 h. Upon completion, the reaction mixture was cooled to RT, and poured into cold water. The resulting solid was collected by filtration, and purified by HPLC to give off-white solid (123 mg, 37%). MS (ESI) m/z 444.1.

### Example 93

### Preparation of N-[4-({[2-(dimethylamino)-7-(hydroxymethyl)quinazolin-4-yl]amino}methyl)phenyl]-4-fluorobenzamide

To a solution of N-[4-({[2-(dimethylamino)-7-formylquinazolin-4-yl]amino}methyl)phenyl]-4-fluorobenzamide (100 mg, 0.23 mmol) in MeOH (5 mL) was added sodium borohydride (NaBH₄) (17 mg, 0.46 mmol). The resulting mixture was stirred at RT for 2 h. The reaction mixture was concentrated under reduced pressure, and the residue was treated with sat. Sodium bicarbonate (NaHCO₃) aqueous solution and extracted with ethyl acetate (EtOAc). The organic phases were washed with brine and dried over (MgSO₄). The solvent was removed and the residue was purified by HPLC and converted to HCl salt to give off-white solid (94 mg, 85%); Mp: 158° C.; MS (ESI) m/z 446.2.

### Example 94

### Preparation of N-[4-({[7-acetyl-2-(dimethylamino)quinazolin-4-yl]amino}methyl)phenyl]-4-fluorobenzamide

To a solution of N-[4-({[2-(dimethylamino)-7-iodoquinazolin-4-yl]amino}methyl)phenyl]-4-fluorobenzamide (400 mg, 0.74 mmol) in DMF (5 mL), was added PdCl₂(PPh₃)₂ (26 mg, 5 mol %) as catalyst, followed by addition of tributyl(1-ethoxyvinyl)tin (0.27 mL, 0.81 mmol). The resulting mixture was heated at 90° C. under nitrogen for 4 h. Upon completion, the reaction mixture was cooled down to RT, and then poured into cold water. The resulting solid was collected by filtration. The solid was dissolved in MeOH (3 mL) and 6N HCl (0.5 mL), and heated at 70° C. for 3 h. The mixture was cooled to RT, and concentrated in vacuum. The resulting residue was purified by flash chromatography (CH₂Cl₂:CH₃OH=90:10) to give off-white solid (168 mg, yield: 50%). Mp: 195° C.; MS (ESI) m/z 458.2.

### Example 95

### Preparation of N-[4-({[2-(dimethylamino)-7-(1-hydroxyethyl)quinazolin-4-yl]amino}methyl)phenyl]-4-fluorobenzamide

To a solution of N-[4-({[7-acetyl-2-(dimethylamino)quinazolin-4-yl]amino}methyl)phenyl]-4-fluorobenzamide (84 mg, 0.18 mmol) in MeOH (5 mL) was added NaBH₄ (14 mg, 0.36 mmol). The resulting mixture was stirred at RT for 6 h. The reaction mixture was concentrated under reduced pressure, and the residue was treated with sat. NaHCO₃ aqueous solution and extracted with EtOAc). The organic phases were washed with brine, and dried over (MgSO₄). The solvent was removed, and the residue was purified by HPLC, and converted to HCl salt to give off-white solid (71 mg, 80%); Mp: 232° C.; MS (ESI) m/z 460.2.

### Example 96

### Preparation of N-{4-[({2-(dimethylamino)-7-[(1E)-prop-1-en-1-yl]quinazolin-4-yl}amino)methyl]phenyl}-4-fluorobenzamide

The compound was prepared from N-[4-({[2-(dimethylamino)-7-iodoquinazolin-4-yl]amino}methyl)phenyl]-4-fluorobenzamide (200 mg, 0.37 mmol) and (E)-tributyl(1-propenyl)tin by following the same procedure as Example 87 (Stille coupling) as off-white solid (HCl salt, 138 mg) in 76% yield. Mp: 268° C.; MS (ESI) m/z 456.1.

### Example 97

### Preparation of N-{4-[({2-(dimethylamino)-7-[(1Z)-prop-1-en-1-yl]quinazolin-4-yl}amino)methyl]phenyl}-4-fluorobenzamide

A glass tube was charged with N-[4-({[2-(dimethylamino)-7-iodoquinazolin-4-yl]amino}methyl)phenyl]-4-fluorobenzamide (200 mg, 0.37 mmol), (Z)-1-propenylboronic acid (48 mg, 0.55 mmol), Pd(PPh₃)₄ (23 mg, 5 mol %), DME (3 mL) and Sat. NaHCO₃ (2 mL). The resulting mixture was heated at 100° C. for 15 min under N₂ in microwave, and then cooled to RT. Filtered, washed with methanol, and the filtrate was concentrated in vacuum to give a solid, which was subjected to HPLC separation. The product was converted to HCl salt to give off-white solid (119 mg, 65%). Mp: 246° C.; MS (ESI) m/z 456.2.

### Example 98

### Preparation of N-[4-({[2-(dimethylamino)-7-(2-formylphenyl)quinazolin-4-yl]amino}methyl)phenyl]-4-fluorobenzamide

The compound was prepared from] N-[4-({[2-(dimethylamino)-7-iodoquinazolin-4-yl]amino}methyl)phenyl]-4-fluorobenzamide (100 mg, 0.18 mmol) and 2-formylphenylboronic acid (32 mg, 0.22 mmol) by following the same procedure as Example 97 (Suzuki coupling) as brown solid (HCl salt, 48 mg) in 48% yield. Mp: 128° C.; MS (ESI) m/z 520.2.

### Example 99

### Preparation of N-[4-({[2-(dimethylamino)-7-(4-formylphenyl)quinazolin-4-yl]amino}methyl)phenyl]-4-fluorobenzamide

The compound was prepared from N-[4-({[2-(dimethylamino)-7-iodoquinazolin-4-yl]amino}methyl)phenyl]-4-fluorobenzamide (100 mg, 0.18 mmol) and 4-formylphenylboronic acid (38 mg, 0.25 mmol) by following the same procedure as Example 97 (Suzuki coupling) as off-white solid (HCl salt, 47 mg) in 47% yield. MS (ESI) m/z 520.2.

### Example 100

### Preparation of N-[4-({[7-(2-chloropyridin-3-yl)-2-(dimethylamino)quinazolin-4-yl]amino}methyl)phenyl]-4-fluorobenzamide

The compound was prepared from N-[4-({[2-(dimethylamino)-7-iodoquinazolin-4-yl]amino}methyl)phenyl]-4-fluorobenzamide (100 mg, 0.18 mmol) and 2-chloropyridin-3-ylboronic acid (40 mg, 0.25 mmol) by following the same procedure as Example 97 (Suzuki coupling) as off-white solid (HCl salt, 56 mg) in 55% yield. MS (ESI) m/z 527.2.

### Example 101

### Preparation of N-[4-({[7-(1-benzofuran-2-yl)-2-(dimethylamino)quinazolin-4-yl]amino}methyl)phenyl]-4-fluorobenzamide

The compound was prepared from N-[4-({[2-(dimethylamino)-7-iodoquinazolin-4-yl]amino}methyl)phenyl]-4-fluorobenzamide (100 mg, 0.18 mmol) and 1-benzofuran-2-ylboronic acid (40 mg, 0.25 mmol) by following the same procedure as Example 97 (Suzuki coupling) as off-white solid (HCl salt, 22 mg) in 21% yield. MS (ESI) m/z 532.2.

### Example 102

### Preparation of N-{4-[({2-(dimethylamino)-7-[(1E)-3,3-dimethylbut-1-en-1-yl]quinazolin-4-yl}amino)methyl]phenyl}-4-fluorobenzamide

The compound was prepared from N-[4-({[2-(dimethylamino)-7-iodoquinazolin-4-yl]amino}methyl)phenyl]-4-fluorobenzamide (150 mg, 0.28 mmol) and (E)-3,3-dimethylbut-1-enylboronic acid (53 mg, 0.42 mmol) by following the same procedure as Example 97 (Suzuki coupling) as off-white solid (HCl salt, 40 mg) in 27% yield. MS (ESI) m/z 498.3.

### Example 103

### Preparation of N-{4-[({2-(dimethylamino)-7-[(1E)-hex-1-en-1-yl]quinazolin-4-yl}amino)methyl]phenyl}-4-fluorobenzamide

The compound was prepared from N-[4-({[2-(dimethylamino)-7-iodoquinazolin-4-yl]amino}methyl)phenyl]-4-fluorobenzamide (120 mg, 0.22 mmol) and (E)-hex-1-enylboronic acid (70 mg, 0.55 mmol) by following the same procedure as Example 97 (Suzuki coupling) as off-white solid (HCl salt, 65 mg) in 55% yield. MS (ESI) m/z 498.3.

### Example 104

### Preparation of N-[4-({[7-cyclopropyl-2-(dimethylamino)quinazolin-4-yl]amino}methyl)phenyl]-4-fluorobenzamide

The compound was prepared from N-[4-({[2-(dimethylamino)-7-iodoquinazolin-4-yl]amino}methyl)phenyl]-4-fluorobenzamide (100 mg, 0.18 mmol) and cyclopropylboronic acid (24 mg, 0.28 mmol) and by following the same procedure as Example 97 (Suzuki coupling) as off-white solid (HCl salt, 32 mg) in 36% yield. MS (ESI) m/z 456.1.

### Example 105

### 6-chloro-N-[4-((1S)-1-{[2-(dimethylamino)-7-iodoquinazolin-4-yl]amino}ethyl)phenyl]nicotinamide

Starting from N-{4-[(1S)-1-aminoethyl]phenyl}-6-chloronicotinamide (220 mg, 0.8 mmol) and 2,4-dichloro-7-iodoquinazoline (285 mg, 0.88 mmol) by following the same procedure as Example 86 (step 5), 6-chloro-N-(4-{(1S)-1-[(2-chloro-7-iodoquinazolin-4-yl)amino]ethyl}phenyl)nicotinamide was isolated as off-white solid (460 mg, 99% yield). MS (ESI) m/z 564.2.

Starting from 6-chloro-N-(4-{(1S)-1-[(2-chloro-7-iodoquinazolin-4-yl)amino]ethyl}phenyl)nicotinamide (430 mg, 0.76 mmol) and dimethylamine hydrochloride (311 mg, 3.8 mmol) and following the same procedure as Example 86 (step 6), 6-chloro-N-[4-((1S)-1-{[2-(dimethylamino)-7-iodoquinazolin-4-yl]amino}ethyl)phenyl]nicotinamide was isolated as off-white solid (395 mg, 85% yield). HRMS: calcd for C₂₄H₂₂ClIN₆O+H+, 573.06611; found (ESI-FTMS, [M+H]¹⁺), 573.06749.

### Example 106

### 6-chloro-N-[4-((1S)-1-{[2-(dimethylamino)-7-vinylquinazolin-4-yl]amino}ethyl)phenyl]nicotinamide

6-chloro-N-[4-((1S)-1-{[2-(dimethylamino)-7-vinylquinazolin-4-yl]amino}ethyl)phenyl]nicotinamide was prepared from 6-chloro-N-[4-((1S)-1-{[2-(dimethylamino)-7-iodoquinazolin-4-yl]amino}ethyl)phenyl]nicotinamide (170 mg, 0.3 mmol) and tributyl(vinyl)tin (105 mg, 0.33 mmol) by following the same procedure as in Example 87 (Stille coupling) to give the product as yellow solid (82 mg, 58% yield). MS (ESI) m/z 473.3; HRMS: calcd for C₂₆H₂₅ClN₆O+H+, 473.18511; found (ESI-FTMS, [M+H]¹⁺), 473.18605.

### Example 107

### 6-chloro-N-{4-[(1S)-1-({2-(dimethylamino)-7-[(1E)-prop-1-en-1-yl]quinazolin-4-yl}amino)ethyl]-phenyl}nicotinamide

6-chloro-N-{4-[(1S)-1-({2-(dimethylamino)-7-[(1E)-prop-1-en-1-yl]quinazolin-4-yl}amino)ethyl]phenyl}nicotinamide was prepared from 6-chloro-N-[4-((1S)-1-{[2-(dimethylamino)-7-iodoquinazolin-4-yl]amino}ethyl)phenyl]nicotinamide (170 mg, 0.3 mmol) and (E)-tributyl(1-propenyl)tin (119 mg, 0.36 mmol) by following the same procedure as in Example 87 (Stille coupling) to give the product as yellow solid (39 mg, 27% yield). MS (ESI) m/z 487.3; HRMS: calcd for C₂₇H₂₇ClN₆O+H+, 487.20076; found (ESI-FTMS, [M+H]¹⁺), 487.20171.

### Example 108

### Preparation of N-[4-({[2-(dimethylamino)-7-ethynylquinazolin-4-yl]amino}methyl)phenyl]-4-fluorobenzamide

To a solution of N-[4-({[2-(dimethylamino)-7-iodoquinazolin-4-yl]amino}methyl)phenyl]-4-fluorobenzamide (150 mg, 0.28 mmol) in DMF (3 mL) were added ethynyltrimethylsilane (80 mg, 0.56 mmol), PdCl₂(PPh₃)₂ (10 mg, 5 mol %), CuI (3 mg, 5 mol %) and Et₃N (0.12 mL, 0.84 mmol). The resulting mixture was stirred at RT under N₂ for 3 h. Upon completion, the reaction mixture was cooled to RT, and poured into cold water. The resulting solid was collected by filtration.

The solid was stirred with KOH (1M in MeOH, 2 mL) at RT overnight. The reaction mixture was adjusted pH to 5-6 by addition of 1N HCl, and then extracted with EtOAc. The combined organic phases was washed with brine, and dried over MgSO₄. The solvent was removed under reduced pressure, and the residue was purified by flash chromatography (EtOAc:CH₂Cl₂=70:30) to give off-white solid, which was treated with 4N HCl dioxane solution to give HCl salt as off-white solid (68 mg, 51%). Mp: 250° C.; MS (ESI) m/z 440.2.

### Example 109

### Preparation of 6-chloro-N-(4-{[(2-chloro-7-iodoquinazolin-4-yl)amino]methyl}phenyl)nicotinamide

The compound was prepared from 2,4-dichloro-7-iodoquinazoline (648 mg, 2 mmol) and N-[4-(aminomethyl)phenyl]-6-chloronicotinamide (522 mg, 2 mmol) by following the same procedure as Example 86 (step 5) as off-white solid (936 mg) in 85% yield; Mp: 315° C.; MS (ESI) m/z 550.1.

### Example 110

### Preparation of 6-chloro-N-[4-({[2-(dimethylamino)-7-iodoquinazolin-4-yl]amino}methyl)phenyl]nicotinamide

Step 1: Starting from 2,4-dichloro-7-iodoquinazoline (648 mg, 2 mmol) and N-[4-(aminomethyl)phenyl]-6-chloronicotinamide (522 mg, 2 mmol) by following the same procedure as Example 87 (step 5), 6-chloro-N-(4-{[(2-chloro-7-iodoquinazolin-4-yl)amino]methyl}phenyl)nicotinamide was isolated as off-white solid (936 mg) in 85% yield; Mp: 315° C.; MS (ESI) m/z 550.1.

Step 2: To a solution of 6-chloro-N-(4-{[(2-chloro-7-iodoquinazolin-4-yl)amino]methyl}phenyl)nicotinamide (400 mg, 0.73 mmol) in DMF (2 mL) was added dimethylamine hydrochloride (279 mg, 3.64 mmol). The mixture was heated at 120° C. for 10 min in microwave, and cooled to RT. The reaction mixture was poured into cold water, and the resulting solid was collected by filtration. After drying, the solid was treated with hot ethanol, then cooled to RT, filtered, and washed with cold ethanol. The title compound was obtained as off-white solid (HCl salt, 375 mg) in 86% yield. Mp: 170° C.; MS (ESI) m/z 559.1.

### Example 11

### Preparation of 6-chloro-N-[4-({[2-(dimethylamino)-7-vinylquinazolin-4-yl]amino}methyl)phenyl]nicotinamide

Method A: The compound was prepared from 6-chloro-N-[4-({[2-(dimethylamino)-7-iodoquinazolin-4-yl]amino}methyl)phenyl]nicotinamide (150 mg, 0.27 mmol) by following the same procedure as Example 87 (Stille coupling) as off-white solid (HCl salt, 61 mg) in 46% yield. Mp: 290° C.; MS (ESI) m/z 459.2.

Method B: Step 1: Synthesis of 2,4-dichloro-7-vinylquinazoline. To a suspension of 7-iodoquinazoline-2,4(1H,3H)-dione (5.0 g, 17.4 mmol) in DMF (20 mL) was added PdCl₂(PPh₃)₂ (609 mg, 5 mol %) as catalyst, followed by addition of tributyl(vinyl)tin (6.1 mL, 20.9 mmol). The resulting mixture was heated at 105° C. under nitrogen for 30 min in microwave. Upon completion, the reaction mixture was cooled down to RT, and then poured into cold water. The resulting solid was collected by filtration, and dried in vacuum to give a brown solid (4.5 g). To the solid was added POCl₃ (40 mL), and the resulting mixture was heated at 115° C. for 3 h. After cooling to RT, most of POCl₃ was removed by distillation under reduced pressure. The residue was poured into ice-water, ammonium hydroxide was added to adjust pH to 5-7. The mixture was extracted several times with CH₂Cl₂, and the combined extracts were washed with brine, and dried over (MgSO₄). The solvent was removed under reduced pressure, and the residue was purified by flash chromatography (EtOAc:Hexane=10:90) to give as off-white solid (2.86 g, 74%); MS (ESI) m/z 224.9.

### Step 2: Synthesis of 6-chloro-N-(4-{[(2-chloro-7-vinylquinazolin-4-yl)amino]methyl}phenyl)nicotinamide

The compound was prepared from 2,4-dichloro-7-vinylquinazoline (400 mg, 1.8 mmol) and N-[4-(aminomethyl)phenyl]-6-chloronicotinamide (522 mg, 2 mmol) by following the same procedure as Example 66 (step 1) as off-white solid (509 mg) in 63% yield. MS (ESI) m/z 450.1.

### Step 3: Synthesis of 6-chloro-N-[4-({[2-(dimethylamino)-7-vinylquinazolin-4-yl]amino}methyl)phenyl]nicotinamide

To a solution of 6-chloro-N-(4-{[(2-chloro-7-vinylquinazolin-4-yl)amino]methyl}phenyl)nicotinamide (250 mg, 0.56 mmol) in DMF (3 mL) was added dimethylamine hydrochloride (227 mg, 2.78 mmol). The mixture was heated at 120° C. for 10 min. in microwave, and cooled to RT. The reaction mixture was poured into cold water, and the resulting solid was collected by filtration. After drying, the solid was treated with hot ethanol. The mixture was cooled to RT, filtered, and washed with cold ethanol. The title compound was obtained as off-white solid (HCl salt, 235 mg) in 85% yield; Mp: 290° C.; MS (ESI) m/z 459.2.

### Example 112

### Preparation of 6-chloro-N-{4-[({2-(dimethylamino)-7-[(1E)-prop-1-en-1-yl]quinazolin-4-yl}amino)methyl]phenyl}nicotinamide

The compound was prepared from 6-chloro-N-[4-({[2-(dimethylamino)-7-iodoquinazolin-4-yl]amino}methyl)phenyl]nicotinamide (150 mg, 0.27 mmol) and (E)-tributyl(1-propenyl)tin (178 mg, 0.54 mmol) by following the same procedure as Example 87 (Stille coupling) as off-white solid (HCl salt, 36 mg) in 26% yield. Mp: 185° C.; MS (ESI) m/z 473.2.

### Example 113

### Preparation of N-[4-({[2-(dimethylamino)quinazolin-4-yl]amino}methyl)phenyl]-4-fluorobenzamide

### Step 1: Synthesis of N-(4-{[(2-chloroquinazolin-4-yl)amino]methyl}phenyl)-4-fluorobenzamide

To a solution of N-[4-(aminomethyl)phenyl]-4-fluorobenzamide (1.70 g, 6.97 mmol) in CH₂Cl₂ (30 mL) was added Et₃N (2.6 mL, 18.5 mmol), followed by addition of 2,4-dichloroquinazoline (1.66 g, 8.38 mmol). The resulting mixture was stirred at RT overnight, during which time a lot of precipitate was formed. The resulting solid was collected by filtration and washed with small amount of EtOAc and water to give the expected product as off-white solid (2.43 g, yield: 86%). MS (ESI) m/z 407.3.

### Step 2: Synthesis of N-[4-({[2-(dimethylamino)quinazolin-4-yl]amino}methyl)phenyl]-4-fluorobenzamide

To a solution of N-(4-{[(2-chloroquinazolin-4-yl)amino]methyl}phenyl)-4-fluorobenzamide (100 mg, 0.25 mmol) in THF (2 mL) was added dimethylamine solution (40% in water, 0.32 mL, 2.5 mmol). The resulting mixture was heated at 100° C. in a sealed tube for 21 h, then cooled to RT. The solvent was evaporated under reduced pressure, and the residue was purified by flash chromatography (CH₂Cl₂:CH₃OH=90:10) to give off-white solid (96 mg, yield: 93%). MS (ESI) m/z 416.2.

### Example 114

### Preparation of N-(4-{[(2-azetidin-1-ylquinazolin-4-yl)amino]methyl}phenyl)-4-fluorobenzamide

The compound was prepared from N-(4-{[(2-chloroquinazolin-4-yl)amino]methyl}phenyl)-4-fluorobenzamide (203 mg, 0.5 mmol) and azetidine hydrochloride (187 mg, 2 mmol) by following the same procedure as Example 66 (step 2) as a white solid (181 mg, 85%). Mp: 249° C.; MS (ESI) m/z 428.3.

### Example 115

### Preparation of N-[4-({[2-(cyclobutylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (120 mg, 0.3 mmol) and cyclobutylamine hydrochloride (323 mg, 3 mmol) by following the same procedure as Example 66 (step 2) as a white solid (77 mg, 58%). MS (ESI) m/z 442.4.

### Example 116

### Preparation of 4-fluoro-N-[4-({[2-(4-methylpiperazin-1-yl)quinazolin-4-yl]amino}methyl)phenyl]benzamide

The compound was prepared from N-(4-{[(2-chloroquinazolin-4-yl)amino]methyl}phenyl)-4-fluorobenzamide (120 mg, 0.3 mmol) and 1-methylpiperazine (0.33 mL, 3 mmol) by following the same procedure as Example 66 (step 2) as a white solid (140 mg, 99%). MS (ESI) m/z 471.4.

### Example 117

### Preparation of 4-fluoro-N-(4-{[(2-morpholin-4-ylquinazolin-4-yl)amino]methyl}phenyl)benzamide

The compound was prepared from N-(4-{[(2-chloroquinazolin-4-yl)amino]methyl}phenyl)-4-fluorobenzamide (120 mg, 0.3 mmol) and morpholine (0.29 mL, 3 mmol) by following the same procedure as Example 66 (step 2) as a white solid (90 mg, 66%). MS (ESI) m/z 458.3.

### Example 118

### Preparation of N-[4-({[2-(ethylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (120 mg, 0.3 mmol) and ethylamine (70% in water, 0.25 mL, 3 mol) by following the same procedure as Example 66 (step 2) as a white solid (97 mg, 78%). MS (ESI) m/z 416.4.

### Example 119

### Preparation of 4-fluoro-N-(4-{[(2-pyrrolidin-1-ylquinazolin-4-yl)amino]methyl}phenyl)benzamide

The compound was prepared from N-(4-{[(2-chloroquinazolin-4-yl)amino]methyl}phenyl)-4-fluorobenzamide (100 mg, 0.25 mmol) and pyrrolidine (0.21 mL, 2.5 mmol) by following the same procedure as Example 66 (step 2) as a white solid (107 mg, 97%). MS (ESI) m/z 442.1.

### Example 120

### Preparation of N-[4-({[2-(cyclopentylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (100 mg, 0.25 mmol) and cyclopentylamine (0.25 mL, 2.5 mmol) by following the same procedure as Example 66 (step 2) as a white solid (55 mg, 48%). MS (ESI) m/z 456.2.

### Example 121

### Preparation of N-[4-({[2-(cyclopropylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (203 mg, 0.5 mmol) and cyclopropylamine (1 mL, 14 mmol) by following the same procedure as Example 66 (step 2) as a white solid (165 mg, 77%). Mp: 189° C.; MS (ESI) m/z 428.3.

### Example 122

### Preparation of N-[4-({[2-(diethylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (100 mg, 0.25 mmol) and diethylamine (0.26 mL, 2.5 mmol) by following the same procedure as Example 66 (step 2) as a white solid (62 mg, 56%). MS (ESI) m/z 444.2.

### Example 123

### Preparation of 4-fluoro-N-(4-{[(2-piperidin-1-ylquinazolin-4-yl)amino]methyl}phenyl)benzamide

The compound was prepared from N-(4-{[(2-chloroquinazolin-4-yl)amino]methyl}phenyl)-4-fluorobenzamide (100 mg, 0.25 mmol) and piperidine (0.25 mL, 2.5 mmol) by following the same procedure as Example 66 (step 2) as a white solid (103 mg, 91%). MS (ESI) m/z 456.5.

### Example 124

### Preparation of 4-fluoro-N-{4-[({2-[(2-furylmethyl)amino]quinazolin-4-yl}amino)methyl]phenyl}benzamide

The compound was prepared from N-(4-{[(2-chloroquinazolin-4-yl)amino]methyl}phenyl)-4-fluorobenzamide (100 mg, 0.25 mmol) and furfurylamine (0.23 mL, 2.5 mmol) by following the same procedure as Example 66 (step 2) as a white solid (100 mg, 87%). MS (ESI) m/z 468.4.

### Example 125

### Preparation of N-[4-({[2-(cyclohexylamino)quinazolin-4-yl]amino}methyl)phenyl]-4-fluorobenzamide

The compound was prepared from N-(4-{[(2-chloroquinazolin-4-yl)amino]methyl}phenyl)-4-fluorobenzamide (100 mg, 0.25 mmol) and cyclohexylamine (0.29 mL, 2.5 mmol) by following the same procedure as Example 66 (step 2) as a white solid (40 mg, 34%). MS (ESI) m/z 470.4.

### Example 126

### Preparation of tert-butyl N-[4-({4-[(4-fluorobenzoyl)amino]benzyl}amino)quinazolin-2-yl]glycinate

The compound was prepared from N-(4-{[(2-chloroquinazolin-4-yl)amino]methyl}phenyl)-4-fluorobenzamide (203 mg, 0.5 mmol) and tert-butyl glycinate (643 mg, 5 mmol) by following the same procedure as Example 66 (step 2) as a white solid (139 mg, 55%). Mp: 96° C.; MS (ESI) m/z 502.3.

### Example 127

### Preparation of N-[4-({4-[(4-fluorobenzoyl)amino]benzyl}amino)quinazolin-2-yl]glycine

To a suspension of tert-butyl N-[4-({4-[(4-fluorobenzoyl)amino]benzyl}amino)quinazolin-2-yl]glycinate (56 mg, 0.9 mmol) in CH₂Cl₂ (3 mL) was added TFA (1 mL) at RT. The mixture was stirred at RT for 3 h, and concentrated in vacuum. The residue was subjected to HPLC separation and then converted to corresponding HCl salt to give the product as off-white solid (46 mg, 88% yield). Mp: 235° C.; MS (ESI) m/z 446.2.

### Example 128

### 6-chloro-N-[4-({[2-(dimethylamino)quinazolin-4-yl]amino}methyl)phenyl]nicotinamide

Step 1: Starting from 2,4-dichloroquinazoline (261 mg, 1.3 mmol) and N-[4-(aminomethyl)phenyl]-6-chloronicotinamide (288 mg, 1.1 mmol) by following the same procedure as Example 66 (step 1), 6-chloro-N-(4-{[(2-chloroquinazolin-4-yl)amino]methyl}phenyl)nicotinamide was isolated as off-white solid (350 mg, 75% yield). MS (ESI) m/z 445.2.

Step 2: Starting from 6-chloro-N-(4-{[(2-chloroquinazolin-4-yl)amino]methyl}phenyl)nicotinamide (150 mg, 0.35 mmol) and dimethylamine (40% in water, 0.23 mL, 1.8 mmol) and following the same procedure as Example 66 (step 2), 6-chloro-N-[4-({[2-(dimethylamino)quinazolin-4-yl]amino}methyl)phenyl]nicotinamide was isolated as off-white solid (80 mg, 53% yield). MS (ESI) m/z 433.3.

At the same time, another product 6-(dimethylamino)-N-[4-({[2-(dimethylamino)quinazolin-4-yl]amino}methyl)phenyl]nicotinamide was also isolated from this reaction as off-white solid (48 mg, 31% yield). MS (ESI) m/z 442.4.

### Example 129

### Preparation of 1-benzyl-N-[4-({[2-(dimethylamino)-7-methylquinazolin-4-yl]amino}methyl)phenyl]piperidine-4-carboxamide

### Step 1: Synthesis of 1-benzyl-N-(4-{[(2-chloro-7-methylquinazolin-4-yl)amino]methyl}phenyl)piperidine-4-carboxamide

To a stirred solution of N-[4-(aminomethyl)phenyl]-1-benzylpiperidine-4-carboxamide (323 mg, 1 mmol) in CH₂Cl₂ (10 mL) was added Et₃N (0.42 mL, 3 mmol), followed by addition of 2,4-dichloro-7-methylquinazoline (254 mg, 1.2 mmol). The resulting mixture was stirred at RT overnight, and then diluted with CH₂Cl₂. The mixture was washed with sat. NaHCO₃ aqueous solution, dried over (MgSO₄). The solvent was removed under reduced pressure, and the residue was purified by flash chromatography (EtOAc:Hexanes:Methanol=70:20:10) to give off-white solid (370 mg, 74%). Mp: 212° C.; MS (ESI) m/z 500.3.

### Step 2: Synthesis of 1-benzyl-N-[4-({[2-(dimethylamino)-7-methylquinazolin-4-yl]amino}methyl)phenyl]piperidine-4-carboxamide

To a solution of 1-benzyl-N-(4-{[(2-chloro-7-methylquinazolin-4-yl)amino]methyl}phenyl)piperidine-4-carboxamide (120 mg, 0.24 mmol) in THF (3 mL) was added dimethylamine solution (40% in water, 0.3 mL, 2.5 mmol). The resulting mixture was heated at 100° C. in a sealed tube for 26 h, then cooled to RT. The solvent was evaporated under reduced pressure, and the residue was purified by HPLC and converted to its HCl salt to give off-white solid (126 mg, yield: 90%). Mp: 100° C.; MS (ESI) m/z 509.3.

### Example 130

### Preparation of 1-benzyl-N-[4-({[7-methyl-2-(methylamino)quinazolin-4-yl]amino}methyl)phenyl]piperidine-4-carboxamide

The compound was prepared from 1-benzyl-N-(4-{[(2-chloro-7-methylquinazolin-4-yl)amino]methyl}phenyl)piperidine-4-carboxamide (120 mg, 0.24 mmol) and methylamine hydrochloride (162 mg, 2.4 mmol) by following the same procedure as Example 129 (step 2) as a white solid (57 mg, 42%). Mp: 128° C.; MS (ESI) m/z 495.4.

### Example 131

### Preparation of N-(4-{[(2-azepan-1-yl-7-methylquinazolin-4-yl)amino]methyl}phenyl)-1-benzylpiperidine-4-carboxamide

The compound was prepared from 1-benzyl-N-(4-{[(2-chloro-7-methylquinazolin-4-yl)amino]methyl}phenyl)piperidine-4-carboxamide (250 mg, 0.5 mmol) and hexamethyleneimine (0.28 mL, 2.5 mmol) by following the same procedure as Example 129 (step 2) as a white solid (168 mg, 53%). Mp: 66° C.; MS (ESI) m/z 563.7.

### Example 132

### Preparation of 1-benzyl-N-[4-({[2-(ethylamino)-7-methylquinazolin-4-yl]amino}methyl)phenyl]piperidine-4-carboxamide

The compound was prepared from 1-benzyl-N-(4-{[(2-chloro-7-methylquinazolin-4-yl)amino]methyl}phenyl)piperidine-4-carboxamide (200 mg, 0.4 mmol) and ethylamine (70% in water, 258 mg, 4 mmol) by following the same procedure as Example 129 (step 2) as a white solid (88 mg, 38%). Mp: 55° C.; MS (ESI) m/z 509.4.

### Example 133

### Preparation of 1-benzyl-N-(4-{[(7-methyl-2-pyrrolidin-1-ylquinazolin-4-yl)amino]methyl}phenyl)piperidine-4-carboxamide

The compound was prepared from 1-benzyl-N-(4-{[(2-chloro-7-methylquinazolin-4-yl)amino]methyl}phenyl)piperidine-4-carboxamide (200 mg, 0.4 mmol) and pyrrolidine (284 mg, 4 mmol) by following the same procedure as Example 129 (step 2) as a white solid (122 mg, 57%). Mp: 96° C.; MS (ESI) m/z 535.3.

### Example 134

### Preparation of N-(4-{[(2-azetidin-1-yl-7-methylquinazolin-4-yl)amino]methyl}phenyl)-1-benzylpiperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and azetidine hydrochloride (28 mg, 0.3 mmol) by following the same procedure as Example 129 (step 2) as off-white solid (19 mg, 59%). MS (ESI) m/z 521.6.

### Example 135

### Preparation of 1-benzyl-N-[4-({[7-methyl-2-(4-pyrrolidin-1-ylpiperidin-1-yl)quinazolin-4-yl]amino}methyl)phenyl]piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 4-pyrrolinylpiperidine (46 mg, 0.3 mmol) by following the same procedure as Example 129 (step 2) as off-white solid (27 mg, 71%). MS (ESI) m/z 618.8.

### Example 136

### Preparation of 1-benzyl-N-[4-({[7-methyl-2-(4-pyrimidin-2-ylpiperazin-1-yl)quinazolin-4-yl]amino}methyl)phenyl]piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 4-pyrimidin-2-ylpiperazine dihydrochloride (71 mg, 0.3 mmol) by following the same procedure as Example 129 (step 2) as off-white solid (19 mg, 50%). MS (ESI) m/z 628.7.

### Example 137

### Preparation of N-(4-{[(2-azetidin-1-yl-7-methylquinazolin-4-yl)amino]methyl}phenyl)-1-benzylpiperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 1-ethylpiperazine (34 mg, 0.3 mmol) by following the same procedure as Example 129 (step 2) as off-white solid (19 mg, 54%). MS (ESI) m/z 578.8.

### Example 138

### Preparation of 1-benzyl-N-{4-[({2-[3-(2-hydroxyethyl)piperazin-1-yl]-7-methylquinazolin-4-yl}amino)methyl]phenyl}piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 2-(piperazin-2-yl)ethanol (39 mg, 0.3 mmol) by following the same procedure as Example 129 (step 2) as off-white solid (33 mg, 91%). MS (ESI) m/z 594.8.

### Example 139

### Preparation of 1-benzyl-N-{4-[({2-[(2-hydroxyethyl)(methyl)amino]-7-methylquinazolin-4-yl}amino)methyl]phenyl}piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 2-methylaminoethanol (23 mg, 0.3 mmol) by following the same procedure as Example 129 (step 2) as off-white solid (29 mg, 87%). MS (ESI) m/z 539.7.

### Example 140

### Preparation of 1-benzyl-N-{4-[({2-[[3-(dimethylamino)propyl](methyl)amino]-7-methylquinazolin-4-yl}amino)methyl]phenyl}-piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and N,N,N′-trimethylpropane-1,3-diamine (35 mg, 0.3 mmol) by following the same procedure as Example 129 (step 2) as off-white solid (26 mg, 73%). MS (ESI) m/z 580.8.

### Example 141

### Preparation of 1-benzyl-N-[4-({[7-methyl-2-(4-methylpiperazin-1-yl)quinazolin-4-yl]amino}methyl)phenyl]piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and 1-methylpiperazine (30 mg, 0.3 mmol) by following the same procedure as Example 129 (step 2) as off-white solid (26 mg, 74%). MS (ESI) m/z 564.7.

### Example 142

### Preparation of 1-benzyl-N-{4-[({2-[benzyl(methyl)amino]-7-methylquinazolin-4-yl}amino)methyl]phenyl}-piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and benzylmethylamine (36 mg, 0.3 mmol) by following the same procedure as Example 129 (step 2) as off-white solid (25 mg, 71%). MS (ESI) m/z 585.6.

### Example 143

### Preparation of 1-benzyl-N-{4-[({7-methyl-2-[(3R)-3-methylpiperazin-1-yl]quinazolin-4-yl}amino)methyl]phenyl}piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and (R)-2-methylpiperazine (30 mg, 0.3 mmol) by following the same procedure as Example 129 (step 2) as off-white solid (34 mg, 98%). MS (ESI) m/z 564.6.

### Example 144

### Preparation of 1-benzyl-N-{4-[({2-[[2-(dimethylamino)ethyl](methyl)amino]-7-methylquinazolin-4-yl}amino)methyl]phenyl}piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and N,N,N′-trimethylethanediamine (31 mg, 0.3 mmol) by following the same procedure as Example 129 (step 2) as off-white solid (26 mg, 76%). MS (ESI) m/z 566.8.

### Example 145

### Preparation of 1-benzyl-N-{4-[({7-methyl-2-[methyl(2-pyridin-2-ylethyl)amino]quinazolin-4-yl}amino)methyl]phenyl}piperidine-4-carboxamide

The compound was prepared from 1-benzyl-N-(4-{[(2-chloro-7-methylquinazolin-4-yl)amino]methyl}phenyl)piperidine-4-carboxamide (30 mg, 0.06 mmol) and methyl(2-pyridinyl)ethylamine (41 mg, 0.3 mmol) by following the same procedure as Example 129 (step 2) as off-white solid (34 mg, 93%). MS (ESI) m/z 600.8.

### Example 146

### Preparation of 1-benzyl-N-[4-({[2-(dimethylamino)quinazolin-4-yl]amino}methyl)phenyl]piperidine-4-carboxamide

Step 1: Starting from N-[4-(aminomethyl)phenyl]-1-benzylpiperidine-4-carboxamide (323 mg, 1 mmol) and 2,4-dichloroquinazoline (238 mg, 1.2 mmol) by following the same procedure as Example 129 (step 1), 1-benzyl-N-(4-{[(2-chloroquinazolin-4-yl)amino]methyl}phenyl)piperidine-4-carboxamide was isolated as off-white solid (456 mg, 94%). MS (ESI) m/z 486.4.

Step 2: Starting from 1-benzyl-N-(4-{[(2-chloroquinazolin-4-yl)amino]methyl}phenyl)piperidine-4-carboxamide (100 mg, 0.2 mmol) and dimethylamine (40% in water, 0.25 mL, 2 mmol) and following the same procedure as Example 129 (step 2), 1-benzyl-N-[4-({[2-(dimethylamino)quinazolin-4-yl]amino}methyl)phenyl]piperidine-4-carboxamide was isolated as off-white solid (60 mg, 59% yield). MS (ESI) m/z 495.4.

### Example 147

### Preparation of 1-benzyl-N-[4-({[2-(methylamino)quinazolin-4-yl]amino}methyl)phenyl]piperidine-4-carboxamide

This compound was prepared from 1-benzyl-N-(4-{[(2-chloroquinazolin-4-yl)amino]methyl}phenyl)piperidine-4-carboxamide (100 mg, 0.2 mmol) and methylamine (2M in THF, 5 mL, 10 mmol) by following the same procedure as Example 129 (step 2) to give the product as off-white solid (50 mg, 51% yield). MS (ESI) m/z 481.4.

### Example 148

### Preparation of 1-benzyl-N-[4-({[2-(dimethylamino)-6-methylquinazolin-4-yl]amino}methyl)phenyl]piperidine-4-carboxamide

Step 1: Starting from N-[4-(aminomethyl)phenyl]-1-benzylpiperidine-4-carboxamide (165 mg, 0.5 mmol) and 2,4-dichloro-6-methylquinazoline (130 mg, 0.6 mmol) by following the same procedure as Example 129 (step 1), 1-benzyl-N-(4-{[(2-chloro-6-methylquinazolin-4-yl)amino]methyl}phenyl)piperidine-4-carboxamide was isolated as off-white solid (139 mg, 55% yield). MS (ESI) m/z 500.5.

Step 2: Starting from 1-benzyl-N-(4-{[(2-chloro-6-methylquinazolin-4-yl)amino]methyl}phenyl)piperidine-4-carboxamide (79 mg, 0.16 mmol) and dimethylamine (40% in water, 0.3 mL, 2.4 mmol) and following the same procedure as Example 129 (step 2), 1-benzyl-N-[4-({[2-(dimethylamino)-6-methylquinazolin-4-yl]amino}methyl)phenyl]piperidine-4-carboxamide was isolated as off-white solid (26 mg, 32% yield). MS (ESI) m/z 509.4.

### Example 149

### Preparation of 1-benzyl-N-[4-({[6-methyl-2-(methylamino)quinazolin-4-yl]amino}methyl)phenyl]piperidine-4-carboxamide

This compound was prepared from 1-benzyl-N-(4-{[(2-chloro-6-methylquinazolin-4-yl)amino]methyl}phenyl)piperidine-4-carboxamide (143 mg, 0.29 mmol) and methyl amine (2M in THF, 5 mL, 10 mmol) and following the same procedure as Example 129 (step 2) as off-white solid (71 mg, 50% yield). MS (ESI) m/z 495.4.

### Example 150

### Preparation of 1-benzyl-N-[4-({[2-(dimethylamino)-7-vinylquinazolin-4-yl]amino}methyl)phenyl]piperidine-4-carboxamide

### Step 1: Synthesis of 1-benzyl-N-[4-({[2-(dimethylamino)-7-iodoquinazolin-4-yl]amino}methyl)phenyl]piperidine-4-carboxamide

To a solution of N-[4-(aminomethyl)phenyl]-1-benzylpiperidine-4-carboxamide (355 mg, 1.1 mmol) in CH₂Cl₂ (10 mL) was added Et₃N (0.28 mL, 3.3 mmol), followed by addition of 2,4-dichloro-7-iodoquinazoline (356 mg, 1.1 mmol). The resulting mixture was stirred at RT for 20 h, during which time a lot of precipitate was formed. The resulting solid was collected by filtration and washed with small amount of EtOAc and water to give the expected product as off-white solid (550 mg, yield: 82%). The resulting solid was dissolved in 3 mL of dimethylamine (2M in THF), and the mixture was heated at 130° C. for 40 min in microwave. The reaction mixture was then concentrated in vacuum, and the residue was treated with ethanol to give the product as off-white solid (520 mg, 93% yield); Mp: 194° C. MS (ESI) m/z 621.2.

### Step 2: Synthesis of 1-benzyl-N-[4-({[2-(dimethylamino)-7-vinylquinazolin-4-yl]amino}methyl)phenyl]piperidine-4-carboxamide

To a solution of 1-benzyl-N-[4-({[2-(dimethylamino)-7-iodoquinazolin-4-yl]amino}methyl)phenyl]piperidine-4-carboxamide (200 mg, 0.32 mmol) in DMF (2 mL), was added PdCl₂(PPh₃)₂ (11 mg, 5 mol %) as catalyst, followed by addition of tributyl(vinyl)tin (0.14 mL, 0.48 mmol). The resulting mixture was heated at 110° C. under nitrogen for 10 min in microwave. Upon completion, the reaction mixture was cooled down to RT, and then poured into cold water. The resulting solid was collected by filtration, and then purified by flash chromatography (CH₂Cl₂:CH₃OH=90:10) to give off-white solid (129 mg, yield: 78%). Mp: 180° C.; MS (ESI) m/z 521.3.

### Example 151

### Preparation of 1-benzyl-N-{4-[({2-(dimethylamino)-7-[(1E)-prop-1-en-1-yl]quinazolin-4-yl}amino)methyl]phenyl}-piperidine-4-carboxamide

This compound was prepared from 1-benzyl-N-[4-({[2-(dimethylamino)-7-iodoquinazolin-4-yl]amino}methyl)phenyl]piperidine-4-carboxamide (200 mg, 0.32 mmol) and (E)-tributyl(1-propenyl)tin (159 mg, 0.48 mmol) by following the same procedure as Example 150 (step 2) as yellow solid (163 mg, yield: 95%). mp 154° C.; HRMS: calcd for C₃₃H₃₈N₆O+H+, 535.31799; found (ESI-FTMS, [M+H]¹⁺), 535.31901.

### Example 152

### Preparation of 1-benzyl-N-{4-[({2-(dimethylamino)-7-[(1E)-3,3-dimethylbut-1-en-1-yl]quinazolin-4-yl}amino)methyl]phenyl}piperidine-4-carboxamide

This compound was prepared from 1-benzyl-N-[4-({[2-(dimethylamino)-7-iodoquinazolin-4-yl]amino}methyl)phenyl]piperidine-4-carboxamide (150 mg, 0.24 mmol) and (E)-3,3-dimethylbut-1-enylboronic acid (62 mg, 0.48 mmol) by following the same procedure as Example 97 (Suzuki coupling) as off-white solid (100 mg, yield: 72%). HRMS: calcd for C₃₆H₄₄N₆O+H+, 577.36494; found (ESI-FTMS, [M+H]¹⁺), 577.36625.

### Example 153

### Preparation of 1-benzyl-N-{4-[({2-(dimethylamino)-7-[(1Z)-prop-1-en-1-yl]quinazolin-4-yl}amino)methyl]phenyl}piperidine-4-carboxamide

This compound was prepared from 1-benzyl-N-[4-({[2-(dimethylamino)-7-iodoquinazolin-4-yl]amino}methyl)phenyl]piperidine-4-carboxamide (150 mg, 0.24 mmol) and (Z)-1-propenylboronic acid (42 mg, 0.48 mmol) by following the same procedure as Example 97 (Suzuki coupling) as off-white solid (38 mg, yield: 30%). HRMS: calcd for C₃₃H₃₈N₆O+H+, 535.31799; found (ESI-FTMS, [M+H]¹⁺), 535.31893.

### Example 154

### Preparation of N-[4-({[2-(dimethylamino)-6-(trifluoromethyl)quinazolin-4-yl]amino}methyl)phenyl]-1-(4-fluorobenzyl)piperidine-4-carboxamide

### Step 1: Synthesis of 2,4-dichloro-6-(trifluoromethyl)quinazoline

This compound was prepared from 3-(trifluoromethyl)aniline (10.0 g, 62 mmol) by following the same procedure as Example 86 to give the product as white solid (3.425 g, 21% yield for 5 steps). HRMS: calcd for C₉H₃Cl₂F₃N₂+H+, 266.96981; found (ESI-FTMS, [M+H]¹⁺), 266.97.

### Step 2: Synthesis of N-[4-({[2-chloro-6-(trifluoromethyl)quinazolin-4-yl]amino}methyl)phenyl]-1-(4-fluorobenzyl)piperidine-4-carboxamide

This compound was prepared from 2,4-dichloro-6-(trifluoromethyl)quinazoline (266 mg, 1 mmol) and N-[4-(aminomethyl)phenyl]-1-(4-fluorobenzyl)piperidine-4-carboxamide (341 mg, 1 mmol) by following the same procedure as Example 129 (step 1) to give the product as off-white solid (516 mg, yield: 80%); MS (ESI) m/z 572.3.

### Step 3 Synthesis of N-[4-({[2-(dimethylamino)-6-(trifluoromethyl)quinazolin-4-yl]amino}methyl)phenyl]-1-(4-fluorobenzyl)piperidine-4-carboxamide

To a solution of dimethylamine (2M in THF, 1 mL) was added N-[4-({[2-chloro-6-(trifluoromethyl)quinazolin-4-yl]amino}methyl)phenyl]-1-(4-fluorobenzyl)piperidine-4-carboxamide (34 mg, 0.06 mmol). The resulting mixture was heated at 130° C. for 30 min in microwave. The mixture was cooled to RT, and subjected to HPLC separation to give the product as off-white solid (23.7 mg, 68% yield). MS (ESI) m/z 581.3.

### Example 155

### Preparation of N-[4-({[2-azetidin-1-yl-6-(trifluoromethyl)quinazolin-4-yl]amino}methyl)phenyl]-1-(4-fluorobenzyl)piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and azetidine hydrochloride (28 mg, 0.3 mmol) by following the same procedure as Example 129 (step 2) as off-white solid (13.5 mg, 38% yield). MS (ESI) m/z 593.3.

### Example 156

### Preparation of 1-(4-fluorobenzyl)-N-[4-({[2-pyrrolidin-1-yl-6-(trifluoromethyl)quinazolin-4-yl]amino}methyl)phenyl]piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and pyrrolidine (21 mg, 0.3 mmol) by following the same procedure as Example 129 (step 2) as off-white solid (25.3 mg, 70% yield). MS (ESI) m/z 607.3.

### Example 157

### Preparation of 1-(4-fluorobenzyl)-N-[4-({[2-(4-pyrimidin-2-ylpiperazin-1-yl)-6-(trifluoromethyl)quinazolin-4-yl]amino}methyl)phenyl]piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and 4-pyrimidin-2-ylpiperazine dihydrochloride (71 mg, 0.3 mmol) by following the same procedure as Example 129 (step 2) as off-white solid (6.1 mg, 15% yield). MS (ESI) m/z 700.3.

### Example 158

### Preparation of N-[4-({[2-(diethylamino)-6-(trifluoromethyl)quinazolin-4-yl]amino}methyl)phenyl]-1-(4-fluorobenzyl)piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and diethylamine (22 mg, 0.3 mmol) by following the same procedure as Example 129 (step 2) as off-white solid (25.7 mg, 70% yield). MS (ESI) m/z 609.2.

### Example 159

### Preparation of N-[4-({[2-(cyclobutylamino)-6-(trifluoromethyl)quinazolin-4-yl]amino}methyl)phenyl]-1-(4-fluorobenzyl)piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and cyclobutylamine hydrochloride (32 mg, 0.3 mmol) by following the same procedure as Example 129 (step 2) as off-white solid (19.9 mg, 55% yield). MS (ESI) m/z 607.2.

### Example 160

### Preparation of 1-(4-fluorobenzyl)-N-[4-({[2-(methylamino-6-(trifluoromethyl)quinazolin-4-yl]amino}methyl)phenyl]piperidine-4-carboxamide

This compound was prepared from N-[4-({[2-chloro-6-(trifluoromethyl)quinazolin-4-yl]amino}methyl)phenyl]-1-(4-fluorobenzyl)piperidine-4-carboxamide (34 mg, 0.06 mmol) and methylamine hydrochloride (20 mg, 0.3 mmol) by following the same procedure as Example 129 (step 2) as off-white solid (15.3 mg, 45% yield). MS (ESI) m/z 567.3.

### Example 161

### Preparation of 1,4-chloro-N-[4-({[2-(dimethylamino)-6-(trifluoromethyl)quinazolin-4-yl]amino}methyl)phenyl]benzamide

Step 1 Synthesis of 4-chloro-N-[4-({[2-chloro-6-(trifluoromethyl)quinazolin-4-yl]amino}methyl)phenyl]benzamide. This compound was prepared from 2,4-dichloro-6-(trifluoromethyl)quinazoline (330 mg, 1.24 mmol) and N-[4-(aminomethyl)phenyl]-4-chlorobenzamide (322 mg, 1.24 mmol) by following the same procedure as Example 66 (step 1) to give the product as off-white solid (494 mg, yield: 81%); MS (ESI) m/z 491.1.

### Step 2 Synthesis of 4-chloro-N-[4-({[2-(dimethylamino)-6-(trifluoromethyl)quinazolin-4-yl]amino}methyl)phenyl]benzamide

To a solution of dimethylamine (2M in THF, 1 mL) was added 4-chloro-N-[4-({[2-chloro-6-(trifluoromethyl)quinazolin-4-yl]amino}methyl)phenyl]benzamide (30 mg, 0.06 mmol). The resulting mixture was heated at 130° C. for 40 min in microwave. The mixture was cooled to RT, and subjected to HPLC separation to give the product as off-white solid (24.6 mg, 82% yield); MS (ESI) m/z 500.2.

### Example 162

### Preparation of N-[4-({[2-azetidin-1-yl-6-(trifluoromethyl)quinazolin-4-yl]amino}methyl)phenyl]-4-chlorobenzamide

This compound was prepared from 4-chloro-N-[4-({[2-chloro-6-(trifluoromethyl)quinazolin-4-yl]amino}methyl)phenyl]benzamide (30 mg, 0.06 mmol) and azetidine hydrochloride (28 mg, 0.3 mmol) by following the same procedure as Example 113 (step 2) as off-white solid (16.4 mg, 53% yield); MS (ESI) m/z 512.2.

### Example 163

### Preparation of 4-chloro-N-[4-({[2-pyrrolidin-1-yl-6-(trifluoromethyl quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and pyrrolidine (21 mg, 0.3 mmol) by following the same procedure as Example 113 (step 2) as off-white solid (21.3 mg, 67% yield). MS (ESI) m/z 526.2.

### Example 164

### Preparation of 4-chloro-N-[4-({[2-(4-pyrimidin-2-ylpiperazin-1-yl)-6-(trifluoromethyl)quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and 4-pyrimidin-2-ylpiperazine dihydrochloride (71 mg, 0.3 mmol) by following the same procedure as Example 113 (step 2) as off-white solid (3.2 mg, 9% yield). MS (ESI) m/z 619.2.

### Example 165

### Preparation of 4-chloro-N-[4-({[2-(diethylamino)-6-(trifluoromethyl)quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and diethylamine (22 mg, 0.3 mmol) by following the same procedure as Example 113 (step 2) as off-white solid (26 mg, 82% yield). MS (ESI) m/z 528.2.

### Example 166

### Preparation of 4-chloro-N-[4-({[2-(cyclobutylamino)-6-(trifluoromethyl)quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and cyclobutylamine hydrochloride (32 mg, 0.3 mmol) by following the same procedure as Example 113 (step 2) as off-white solid (3.7 mg, 12% yield). MS (ESI) m/z 526.2.

### Example 167

### Preparation of 4-chloro-N-[4-({[2-(methylamino)-6-(trifluoromethyl)quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and methylamine hydrochloride (20 mg, 0.3 mmol) by following the same procedure as Example 113 (step 2) as off-white solid (8.2 mg, 28% yield). MS (ESI) m/z 486.2.

### Example 168

### Preparation of 4-chloro-N-[4-({[2-[(2-furylmethyl)amino]-6-(trifluoromethyl)quinazolin-4-yl]amino}methyl)phenyl]benzamide

This compound was prepared from 4-chloro-N-[4-({[2-chloro-6-(trifluoromethyl)quinazolin-4-yl]amino}methyl)phenyl]benzamide (30 mg, 0.06 mmol) and furfurylamine (29 mg, 0.3 mmol) by following the same procedure as Example 113 (step 2) as off-white solid (26 mg, 79% yield). MS (ESI) m/z 552.2.

### Example 169

### Preparation of 1,N-(4-((2-dimethylamino)-6-(5-(dimethylamino)pyridine-2-yl)quinazolin-4-ylamino)methyl)phenyl)-4-fluorobenzamide

N-(4-((2-dimethylamino)-6-(5-(dimethylamino)pyridine-2-yl)quinazolin-4-ylamino)methyl)phenyl)-4-fluorobenzamide was prepared starting from N-(4-((2-(dimethylamino)-6-iodoquinazolin-4-ylamino)methyl)phenyl)-4-fluorobenzide (0.15 g, 0.36 mmol), 6-(dimethylamino)pyridine-3-ylboronic acid (0.119 g, 0.71 mmol) tetrakis(triphenylphosphine)palladium (0) (50 mg, 0.0551 mmol), Toluene (8 mL), methanol (2 mL) and sodium carbonate (2.0 M solution) (4 mL). The reaction mixture was refluxed for 4 hours, and then cooled to room temperature. The mixture was partioned between chloroform (150 mL) and water (150 mL). The water layer was extracted twice with chloroform (150 mL). The combined organic layer was dried over magnesium sulfate. The residue was chromatographed on silica gel using ethyl acetate/MeOH (20:1) to yield the final product 112 mg (yield 58%). MS (ESI) m/z 536.1; MS (ESI) m/z 268.6.

### Example 170

### Preparation of 1,4-((2-dimethylamino)-6-(3-(dimethylamino)phenyl)quinazolin-4-ylamino)methyl)N-4-fluorophenyl)benzamide

4-((2-dimethylamino)-6-(3-(dimethylamino)phenyl)quinazolin-4-ylamino)methyl)N-4-fluorophenyl)benzamide was prepared starting from N-(4-((2-(dimethylamino)-6-iodoquinazolin-4-ylamino)methyl)phenyl)-4-fluorobenzamide (0.53 g, 0.98 mmol), 3-(dimethylamino)phenylboronic acid (0.16 g, 0.98 mmol) tetrakis(triphenylphosphine)palladium (0) (50 mg, 0.0551 mmol), Toluene (8 mL), methanol (2 mL), sodium carbonate (2.0 M solution) (4 mL). The reaction mixture was refluxed for 4 hours, and then cooled to room temperature. The mixture was partioned between chloroform (150 mL) and water (150 mL). The combined organic layer was dried with magnesium sulfate. The residue was chromatographed on silica gel using a ethyl acetate/MeOH (20:1) as an eluent to yield the final product 62 mg (yield 12%). MS (ESI) m/z 535.3.

### Example 171

### Preparation of 1,Z-(4-((2-dimethylamino)-6-(styrylquinazolin-4-ylamino)-N-(4-fluorophenyl)benzamide

Z-(4-((2-dimethylamino)-6-(styrylquinazolin-4-ylamino)-N-(4-fluorophenyl)benzamide was prepared starting from N-(4-((2-(dimethylamino)-6-iodoquinazolin-4-ylamino)methyl)phenyl)-4-fluorobenzamide, (0.36 g, 0.66 mmol), (Z)-styrylboronic acid (97 mg, 0.66 mmol) tetrakis(triphenylphosphine)palladium (0) (50 mg, 0.0551 mmol), Toluene (8 mL), methanol (2 mL), sodium carbonate (2.0 M solution) (4 mL). The reaction mixture was refluxed for 4 hours, and then cooled to room temperature. The mixture was partioned between chloroform (150 mL) and water (150 mL). The water layer was extracted twice with chlororm (150 mL). The combined organic layer was dried with magnesium sulfate. The residue was chromatographed on silica gel using a Hexanes/Ethyl acetate (1:1) as an eluent to yield the final product 62 mg (yield 15%). MS (ESI) m/z 518.3.

### Example 172

### Preparation of 1,4-((2-dimethylamino)-6-(3-vinylphenyl)quinazolin-4-ylamino)methyl)-N-(4-fluorophenyl)benzamide

4-((2-dimethylamino)-6-(3-vinylphenyl)quinazolin-4-ylamino)methyl)-N-(4-fluorophenyl)benzamide was prepared starting from N-(4-((2-(dimethylamino)-6-iodoquinazolin-4-ylamino)methyl)phenyl)-4-fluorobenzamide (0.111 g, 0.20 mmol), tributyl(vinyl)stannane (65 mg, 0.2 mmol) PdCl₂(PPh₃)₂ (50 mg, 0.071 mmol), DMF (20 mL). The reaction mixture was refluxed for 4 hours. The solvent was removed under reduced pressure and the residue partioned between chloroform (150 mL) and water (150 mL). The water layer was extracted twice with chloroform (150 mL). The combined organic layer was dried with magnesium sulfate. The residue was chromatographed on silica gel using CHCl₃/MeOH (20:2) as an eluent to yield the final product 62 mg (yield 68%). MS (ESI) m/z 442.2.

### Example 173

### Preparation of 1,E-(4-((2-dimethylamino)-6-(4-styrylphenyl)quinazolin-4-ylamino)methyl-N-(4-fluorophenyl)benzamide

E-(4-((2-dimethylamino)-6-(4-styrylphenyl)quinazolin-4-ylamino)methyl-N-(4-fluorophenyl)benzamide was prepared starting from N-(4-((2-(dimethylamino)-6-iodoquinazolin-4-ylamino)methyl)phenyl)-4-fluorobenzamide (0.41 g, 0.75 mmol), (E)-styrylboronic acid (111 mg, 0.75 mmol) tetrakis(triphenylphosphine)palladium (0) (50 mg, 0.0551 mmol), Toluene (8 mL), methanol (2 mL), sodium carbonate (2.0 M solution) (4 mL). The reaction mixture was refluxed for 4 hours, and then cooled to room temperature. The mixture was partioned between chloroform (150 mL) and water (150 mL). The combined organic layer was dried with magnesium sulfate. The residue was chromatographed on silica gel using Hexanes/Ethyl acetate (1:1) as an eluent to yield the final product 40 mg (yield 10%). MS (ESI) m/z 518.3.

### Example 174

### Preparation of, E-4-((2-dimethylamino)-6-(prop-1-enyl)quinazolin-4-ylamino)methyl-N-(4-fluorophenyl)benzamide

E-4-((2-dimethylamino)-6-(prop-1-enyl)quinazolin-4-ylamino)methyl-N-(4-fluorophenyl)benzamide was prepared starting from N-(4-((2-(dimethylamino)-6-iodoquinazolin-4-ylamino)methyl)phenyl)-4-fluorobenzamide (0.5 g, 0.92 mmol), (E)-tributyl(prop-1-enyl)stannane (0.30 g, 0.90 mmol) PdCl₂(PPh₃)₂ (50 mg, 0.071 mmol), DMF (20 mL). The reaction mixture was refluxed for 4 hours, and then cooled to room temperature. The mixture was partioned between chloroform (150 mL) and water (150 mL). The water layer was extracted twice with chlororm (150 mL). The combined organic layer was dried with magnesium sulfate. The residue was chromatographed on silica gel using 50% CH₂Cl₂/EtOAc and 5% MeOH as en eluent to give 100 mg (24% Yield) of the final product. MS (ESI) m/z 456.3.

### Example 175

### Preparation of E-(4-((2-dimethylamino)-6-(hex-1-enyl)quinazolin-4-ylamino)methyl)-N-(4-fluorophenyl)benzamide

E-(4-((2-dimethylamino)-6-(hex-1-enyl)quinazolin-4-ylamino)methyl)-N-(4-fluorophenyl)benzamide was prepared starting from N-(4-((2-(dimethylamino)-6-iodoquinazolin-4-ylamino)methyl)phenyl)-4-fluorobenzamide (0.50 g, 0.92 mmol), trans-1-hexen-boronic acid (120 mg, 0.93 mmol) (tetrakis(triphenylphosphine)palladium (0) (50 mg, 0.0551 mmol), Toluene (8 mL), methanol (2 mL), sodium carbonate (2.0 M solution) (4 mL). The reaction mixture was refluxed for 4 hours, and then cooled to room temperature. The mixture was partioned between chloroform (150 mL) and water (150 mL). The water layer was extracted twice with chlororm (150 mL). The combined organic layer was dried with magnesium sulfate. The residue was chromatographed on silica gel using Hexanes/Ethyl acetate (1:1) as an eluent to yield the final product 80 mg (63% yield). MS (ESI) m/z 498.4.

### Example 176

### Preparation of (E)-N-{4-[({2-(dimethylamino)-6-(3,3-dimethylbut-1-enyl)quinazolin-4-ylamino)methyl)-N-(4-fluorophenyl)benzamide

(E)-N-{4-[({2-(dimethylamino)-6-(3,3-dimethylbut-1-enyl)quinazolin-4-ylamino)methyl)-N-(4-fluorophenyl)benzamide, E-(4-((2-dimethylamino)-6-(4-styrylphenyl)quinazolin-4-ylamino)methyl-N-(4-fluorophenyl)benzamide was prepared starting from N-(4-((2-(dimethylamino)-6-iodoquinazolin-4-ylamino)methyl)phenyl)-4-fluorobenzamide (0.50 g, 0.92 mmol), (E)-3,3-dimethylbut-1-enylboronic acid (118 mg, 0.92 mmol) (tetrakis(triphenylphosphine)palladium (0) (50 mg, 0.0551 mmol), Toluene (8 mL), methanol (2 mL), sodium carbonate (2.0 M solution) (4 mL). The reaction mixture was refluxed for 4 hours, and then cooled to room temperature. The mixture was partioned between chloroform (150 mL) and water (150 mL). The water layer was extracted twice with chlororm (150 mL). The combined organic layer was dried with magnesium sulfate. The residue was chromatographed on silica gel using a eluente Hexanes/Ethyl acetate (1:1) to yield the final product 50 mg (yield 11%). MS (ESI) m/z 498.4.

### Preparation of ethyl {[(4-isopropylphenyl)amino]carbonothioyl}carbamate

A mixture of 4-isopropylaniline (3.76, 27.85 mol) and ethoxycarbonyl thioisocyanate (3.65 g, 27.85 mmol) was stirred in dichlormethane at room temperature for 3 hours. The separated solid was filtered and washed with hexanes, to give 5.17 g (70% yield) of the product. MS (ESI) m/z 267.2.

### Preparation of ethyl {(E)-(ethylthio)[(4-isopropylphenyl)imino]methyl}carbamate

A mixture of ethyl {[(4-isopropylphenyl)amino]carbonothioyl}carbamate) (5.17 g, 17.58 mmol), dry acetone 300 (mL), potassium carbonate (13.41 g, 97.17 mmol) was stirred for five minutes at room temperature. To the reaction mixture was added ethyliodide (3.15 g, 20.19 mmol) and stirred at room temperature for 24 hours. The reaction mixture was filtered and the solvent was removed. The residue was extracted with dichloromethane (200 mL) and washed with water. The organic layer was dried over magnesium sulfate; filtered and concentrated to give 4.5 g (93%) of the product. MS (ESI) m/z 295.2.

### Preparation of (2-(ethylthio)-6-isopropylquinazolin-4(3H)-one)

A mixture of ethyl {(E)-(ethylthio)[(4-isopropylphenyl)imino]methyl}carbamate (4.5 g, 15.30 mmol), diphenylether 40 (mL) was heated to 235° C. for 2 hours. The mixture was cooled at room temperature and the separated solid was filtered and washed with plenty of hexanes. The residue was dried at room temperature to give 2.67 g (70% yield) of the product. MS (ESI) m/z 249.3.

### Preparation of ethyl {[(2-isopropylphenyl)amino]carbonothioyl}carbamate

A mixture of 2-isopropyaniline (3.76 g, 27.85 mmol) and ethoxycarbonyl thioisocyanate (3.65 g, 27.85 mmol), was stirred in dichloromethane at room temperature for 3 hours. At the end, reaction mixture was concentrated and the separated solid was washed with hexanes and dried to give 5.17 g (70% yield) of the product. MS (ESI) m/z 267.2.

### Preparation of ethyl {(E)-(ethylthio)[(2-isopropylphenyl)imino]methyl}carbamate

A mixture of ethyl {[(2-isopropylphenyl)amino]carbonothioyl}carbamate) (10 g, 35.16 mmol), dry acetone 500 (mL), potassium carbonate (26.82 g, 0.194 mol) was stirred for five minutes at room temperature. To the reaction mixture was added ethyliodide (6.3 g, 40.38 mmol) then stirred at room temperature for 24 hours. The reaction mixture was filtered and the solvent was removed. The residue was extracted with dichloromethane (200 mL) and washed with water. The organic layer was dried over magnesium sulfate; filtered and concentrated to give 9.0 g (93%) of the product. MS (ESI) m/z 295.2.

### Preparation of 2-(ethylthio)-8-isopropylquinazolin-4(3H)-one

A mixture of ethyl {(E)-(ethylthio)[(2-isopropylphenyl)imino]methyl}carbamate) (9.0 g, 30.6 mmol), diphenylether 40 (mL) was heated to 235° C. for 2 hours. The mixture was cooled at room temperature and the separated solid was washed with plenty of hexanes. The residue was dried at room temperature to give 6.8 g (89% yield) of the 2-(ethylthio)-8-isopropylquinazolin-4(3H)-one. MS (ESI) m/z 249.3.

### Preparation of ethyl {[(3-fluoro-2-methylphenyl)amino]carbonothioyl}carbamate

A mixture of 3-fluoro-2-methyl-aniline (10.0 g, 80.0 mmol), ethoxycarbonyl thioisocyanate (10.48 g, 80.0 mmol), dichloromethane (400 mL) was stirred at room temperature for 3 hours. At the end, reaction mixture was concentrated and the separated solid was washed with hexanes, dried to give ethyl {[(3-fluoro-2-methylphenyl)amino]carbonothioyl}carbamate 15.0 g (76% yield). MS (ESI) m/z 257.1.

### Preparation of ethyl {(ethylthio)[(3-fluoro-2-methylphenyl)amino]methyl}carbamate

A mixture of ethyl {[(3-fluoro-2-methylphenyl)amino]carbonothioyl}carbamate) (10.0 g, 39.06 mmol), dry acetone 350 (mL), potassium carbonate (13.41 g, 97.17 mmol) was stirred for five minutes at room temperature. To the reaction mixture was added ethyliodide (6.34 g, 40.0 mmol) then stirred at room temperature for 24 hours. At the end, reaction mixture was filtered and concentrated to give 10.81 g (97%) of the product. MS (ESI) m/z 285.1.

### Preparation of 2-(ethylthio)-7-fluoro-8-methylquinazolin-4(3H)-one

A mixture of ethyl {(ethylthio)[(3-fluoro-2-methylphenyl)amino]methyl}carbamate) (10 g, 34.97 mmol), diphenylether 40 (mL) was heated to 235° C. for 2 hours. The mixture was cooled at room temperature and the separated solid was filtered and washed with plenty of hexanes. The residue was dried at room temperature to give 7.0 g (84% yield) of the product. MS (ESI) m/z 239.1.

### Preparation of 8-isopropyl-3,4-dihydroquinazoline-2,4-diol

A mixture of 2-(ethylthio)-8-isopropylquinazolin-4(3H)-one) (3.8 g, 15.32 mmol), HCl 6N (150 mL), Ethanol (150 mL) was refluxed for 24 hours. The solvent was removed and the mixture was adjusted to pH 6.0. by using NH₄OH solution. The precipitate was filtered, dried to give 3.0 g (95%) of the product. MS (ESI) m/z 203.

### Preparation of ethyl {[(3-isopropylphenyl)amino]carbonothioyl}carbamate

A mixture of 3-isopropylaniline (5.15 g, 38.19 mmol) and ethoxycarbonyl thioisocyanate (5.0 g, 38.16 mmol) was stirred in dichloromethane (250 mL) at room temperature for 3 hours. The reaction mixture was concentrated and the separated solid was washed with hexanes, dried to give (ethyl {[(3-isopropylphenyl)amino]carbonothioyl}carbamate; 7.8 g (78% yield). MS (ESI) m/z 267.4.

### Preparation of ethyl {(ethylthio)[(3-isopropylphenyl)amino]methyl}carbamate

A mixture of ethyl {[(3-isopropylphenyl)amino]carbonothioyl}carbamate) (5.17 g, 17.58 mmol), dry acetone 300 (mL), potassium carbonate (13.41 g, 97.17 mmol) was stirred for five minutes at room temperature. To the reaction mixture was added ethyliodide (3.15 g, 20.19 mmol) and then stirred at room temperature for 24 hours.

The reaction mixture was filtered and concentrated. The residue was combined with water 200 (mL) and extracted with dichloromethane (200 mL) and dried over magnesium sulfate to give 4.5 g (93%) of the product. MS (ESI) m/z 295.1.

### Preparation of 2-(ethylthio)-7-isopropylquinazolin-4(3H)-one

A mixture of ethyl {(ethylthio)[(3-isopropylphenyl)amino]methyl}carbamate (4.5 g, 15.30 mmol), diphenylether 40 (mL) was heated to 235° C. for 2 hours. The mixture was cooled to room temperature and the solid was washed with plenty of hexanes. The residue was dried at room temperature to give 2.67 g (70% yield) of the product 2-(ethylthio)-7-isopropylquinazolin-4(3H)-one was isolated as brown solid. MS (ESI) m/z 249.2.

### Preparation of (N-[4-(aminomethyl)phenyl]-N′-(4-bromophenyl)urea

A mixture of 4-bromophenyl isocyanate (5.0 g, 25.22 mmol) in dichloromethane (400 mL), (4-amino-benzyl)-carbamic acid tert-butyl ester (5.6 g, 25.2 mmol) was stirred at room temperature for 24 hours. At the end, reaction mixture was concentrated and the residue was triturated with diethylether and the precipitate was filtered. The crude solid was taken up in TFA 100 (mL) and stirred at room temperature overnight. The mixture was poured over ice cold water and the pH was adjusted to 10, using 10 N. sodium hydroxide (NaOH). The product was extracted with chloroform and concentrated to give 2.0 g (24% yield) of the product. MS (ESI) m/z 322.

### Preparation of N-[4-(aminomethyl)phenyl]-N′-(4-fluorophenyl)urea

A mixture of 4-fluorophenyl isocyanate (5.0 g, 36.5 mmol) in dichloromethane (400 mL), (4-amino-benzyl)-carbamic acid tert-butyl ester (8.10 g, 36.5 mmol) was stirred at room temperature for 24 hours. At the end, reaction mixture was concentrated and the residue was triturated with diethylether and the precipitate was filtered. The crude solid was taken up in TFA 100 (mL) and stirred at room temperature overnight. The mixture was poured over ice cold water and the pH was adjusted to 10, using 10 N. NaOH. The product was extracted with chloroform and concentrated to give 2.4 g (25% yield) of the product. MS (ESI) m/z 258.1.

### Preparation of N-[4-(aminomethyl)phenyl]-N′-(4-chlorophenyl)urea

A mixture of 4-chlorophenyl isocyanate (5.0 g, 32.6 mmol) in dichloromethane (400 mL), (4-amino-benzyl)-carbamic acid tert-butyl ester (7.2 g, 32.6 mmol) was stirred at room temperature for 24 hours. At the end, reaction mixture was concentrated and the residue was triturated with diethylether and the precipitate was filtered. The crude solid was taken up in TFA 100 (mL) and stirred at room temperature overnight. The mixture was poured over ice cold water and the pH was adjusted to 10, using 10 N. NaOH. The product was extracted with chloroform and concentrated to give 3.1 g (34% yield) of the product. MS (ESI) m/z 276.5.

### Example 177

### Preparation of 1 N-(4-chlorophenyl)-N′-[4-({[2-(dimethylamino)-7-methylquinazolin-4-yl]amino}methyl)phenyl]urea

Step 1: To a stirred solution of 7-methyl-2,4-dichloroquinazoline (800 mg, 3.7 mmol) and N-[4-(aminomethyl)phenyl]-N′-(4-chlorophenyl)urea (1030 mg, 3.7 mmol) in DMF (25 mL) Et₃N (15 mL) was added at room temperature and continued for 5 h. The reaction mixture was concentrated and water (100 mL) was added. Separated solid was filtered and washed with water. The separated solid was suspended in diethylether and filtered. Yield: 1300 mg, 77%.

Step 2: A mixture of N-(4-chlorophenyl)-N′-[4-({[2-(chloro)-7-methylquinazolin-4-yl]amino}methyl)phenyl]urea) (1000 mg, 2.2 mmol) and dimethylamine hydrochloride (20 g) in THF/Isopropanol (1:1) 500 (mL) was refluxed for 2 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 330 mg (33%) of the product was isolated. MS (ESI) m/z 461.2.

### Example 178

### Preparation of N-(4-chlorophenyl)-N′-[4-({[2-(dimethylamino)-6-methylquinazolin-4-yl]amino}methyl)phenyl]urea)

Step 1: To a stirred solution of 6-methyl-2,4-dichloroquinazoline (390 mg, 1.83 mmol) and N-[4-(aminomethyl)phenyl]-N′-(4-chlorophenyl)urea (470 mg, 1.81 mmol) in DMF (25 mL) Et₃N (7 mL) was added at room temperature and continued for 5 h. The reaction mixture was concentrated and water (100 mL) was added. Separated solid was filtered and washed with water. The separated solid was suspended in diethylether and filtered. Yield: 300 mg, 46%.

Step 2: A mixture of N-(4-chlorophenyl)-N′-[4-({[2-(chloro)-6-methylquinazolin-4-yl]amino}methyl)phenyl]urea) (800 mg, 1.7 mmol) and dimethylamine hydrochloride (10 g) in THF/Isopropanol (1:1) 500 (mL) was refluxed for 2 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 384 mg (47%) of the product was isolated. MS (ESI) m/z 461.2.

### Example 179

### Preparation of 1 (N-(4-bromophenyl)-N′-[4-({[2-(dimethylamino)-7-methylquinazolin-4-yl]amino}methyl)phenyl]urea)

Step 1: To a stirred solution of 7-methyl-2,4-dichloroquinazoline (300 mg, 1.40 mmol) and N-[4-(aminomethyl)phenyl]-N′-(4-bromophenyl)urea (450 mg, 1.4 mmol) in DMF (25 mL) Et₃N (7 mL) was added at room temperature and continued for 5 h. The reaction mixture was concentrated and water (100 mL) was added. Separated solid was filtered and washed with water. The separated solid was suspended in diethylether and filtered. Yield: 200 mg, 28%.

Step 2: A mixture of N-(4-bromophenyl)-N′-[4-({[2-(chloro)-7-methylquinazolin-4-yl]amino}methyl)phenyl]urea) (500 mg, 1 mmol) and dimethylamine hydrochloride (10 g) in THF/Isopropanol (1:1) 500 (mL) was refluxed for 2 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 100 mg (20%) of the product was isolated. MS (ESI) m/z 506.3.

### Example 180

### Preparation of 1 N-[4-({[2-(dimethylamino)-7-methylquinazolin-4-yl]amino}methyl)phenyl]-N′-(4-fluorophenyl)urea)

Step 1: To a stirred solution of 7-methyl-2,4-dichloroquinazoline (1230 mg, 5.8 mmol) and N-[4-(aminomethyl)phenyl]-N′-(4-fluorophenyl)urea (1500 mg, 5.8 mmol) in DMF (30 mL) Et₃N (10 mL) was added at room temperature and continued for 12 h. The reaction mixture was concentrated and water (100 mL) was added. Separated solid was filtered and washed with water. The separated solid was suspended in diethylether and filtered. Yield: 1100 mg, 44%.

Step 2: A mixture of N-(4-fluorophenyl)-N′-[4-({[2-(chloro)-7-methylquinazolin-4-yl]amino}methyl)phenyl]urea) (1000 mg, 2.3 mmol) and dimethylamine hydrochloride (16 g) in THF/Isopropanol (1:1) 500 (mL) was refluxed for 5 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 100 mg (20%) of the product was isolated. MS (ESI) m/z 445.3.

### Example 181

### Preparation of 1 N-[4-({[2-(dimethylamino)-6-methylquinazolin-4-yl]amino}methyl)phenyl]-N′-(4-fluorophenyl)urea)

Step 1: To a stirred solution of 6-methyl-2,4-dichloroquinazoline (400 mg, 1.8 mmol) and N-[4-(aminomethyl)phenyl]-N′-(4-fluorophenyl)urea (480 mg, 1.8 mmol) in DMF (20 mL) Et₃N (10 mL) was added at room temperature and continued for 5 h. The reaction mixture was concentrated and water (100 mL) was added. Separated solid was filtered and washed with water. The separated solid was suspended in diethylether and filtered. Yield: 300 mg, 37%.

Step 2: A mixture of N-(4-fluorophenyl)-N′-[4-({[2-(chloro)-6-methylquinazolin-4-yl]amino}methyl)phenyl]urea) (300 mg, 0.69 mmol) and dimethylamine hydrochloride (10 g) in THF/Isopropanol (1:1) 500 (mL) was refluxed for 12 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 150 mg (50%) of the product was isolated. MS (ESI) m/z 445.3.

### Example 182

### Preparation of 1 N-[4-({[2-(dimethylamino)quinazolin-4-yl]amino}methyl)phenyl]-N′-(4-fluorophenyl)urea

Step 1: To a stirred solution of 2,4-dichloroquinazoline (710 mg, 3.6 mmol) and N-[4-(aminomethyl)phenyl]-N′-(4-fluorophenyl)urea (920 mg, 3.6 mmol) in DMF (20 mL) Et₃N (10 mL) was added at room temperature and continued for 5 h. The reaction mixture was concentrated and water (100 mL) was added. Separated solid was filtered and washed with water. The separated solid was suspended in diethylether and filtered. Yield: 1000 mg, 66%.

Step 2: A mixture of N-(4-fluorophenyl)-N′-[4-({[2-(chloro)-quinazolin-4-yl]amino}methyl)phenyl]urea) (1000 mg, 2.4 mmol) and dimethylamine hydrochloride (15 g) in THF/Isopropanol (1:1) 500 (mL) was refluxed for 72 hours, and then cooled to room temperature. Half of solvent volume was evaporated and the mixture was partioned between chloroform (200 mL) and water (200 mL). The water layer was extracted three with chlororm (200 mL). The combined organic layer was dried with magnesium sulfate, filtered. The solvent was evaporated and the residue was washed with plenty of ethyl acetate to give 80 mg (8%) of the product was isolated. MS (ESI) m/z 431.3.

### Example 183

### Preparation of 1 6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide

6-chloro-N-[4-({[8-methyl-2-(methylamino)quinazolin-4-yl]amino}methyl)phenyl]nicotinamide was prepared starting from 8-methyl-2,4-dichloroquinazoline, 4-aminobenzylamine and 6-chloronicotinoylchloride by following the procedure A (step 1). The intermediate product from the step 1 was aminated using monomethylamine to yield the final product. Starting from (544 mg, 2.5 mmol) of 8-methyl-2,4-dichloroquinazoline, 250 mg (Yield, 23%) of the final product was isolated. MS (EST) m/z 433.2. During this reaction 14% of 6-(methylamino)-N-[4-({[8-methyl-2-(methylamino)quinazolin-4-yl]amino}methyl)phenyl]nicotinamide was also isolated. MS (ESI)/Z 428.3.

### Example 184

### Preparation of 6-chloro-N-[4-({[2-(methylamino)-6-nitroquinazolin-4-yl]amino}methyl)phenyl]nicotinamide

6-chloro-N-[4-({[2-(methylamino)-6-nitroquinazolin-4-yl]amino}methyl)phenyl]nicotinamide was prepared starting from 6-nitro-2,4-dichloroquinazoline, 4-aminobenzylamine and 6-chloronicotinoylchloride by following the procedure A (step 1). The intermediate product from the step 1 was aminated using monomethylamine to yield the final product. Starting from (500 mg, 2.05 mmol) of 6-nitro-2,4-dichloroquinazoline, (400 mg, Yield, 42%) of the final product was isolated. MS (ESI) m/z 464.1; mp 305-307° C.

### Example 185

### Preparation of 6-chloro-N-[4-({[2-(methylamino)-8-nitroquinazolin-4-yl]amino}methyl)phenyl]nicotinamide

6-chloro-N-[4-({[2-(methylamino)-8-nitroquinazolin-4-yl]amino}methyl)phenyl]nicotinamide was prepared starting from 8-nitro-2,4-dichloroquinazoline, 4-aminobenzylamine and 6-chloronicotinoylchloride by following the procedure A (step 1). The intermediate product from the step 1 was aminated using monomethylamine to yield the final product. Starting from (1000 mg, 4.14 mmol) of 8-nitro-2,4-dichloroquinazoline, (400 mg, Yield, 45%) of the final product was isolated. MS (ESI) m/z 464.1.

### Example 186

### Preparation of 4-fluoro-N-[4-({[2-(methylamino)-6-nitroquinazolin-4-yl]amino}methyl)phenyl]benzamide

4-fluoro-N-[4-({[2-(methylamino)-6-nitroquinazolin-4-yl]amino}methyl)phenyl]benzamide was prepared starting from 6-nitro-2,4-dichloroquinazoline, 4-aminobenzylamine and 4-fluorobenzoyl chloride by following the procedure A (step 1). The intermediate product from the step 1 was aminated using monomethylamine to yield the final product. Starting from (1000 mg, 4.12 mmol) of 6-nitro-2,4-dichloroquinazoline, 210 mg (Yield, 20%) of the final product was isolated. MS (ESI) m/z 447.3.

### Example 187

### Preparation of N-[4-({[2-(dimethylamino)-6-nitroquinazolin-4-yl]amino}methyl)phenyl]-4-fluorobenzamide

N-[4-({[2-(dimethylamino)-6-nitroquinazolin-4-yl]amino}methyl)phenyl]-4-fluorobenzamide was prepared starting from 6-nitro-2,4-dichloroquinazoline, 4-aminobenzylamine and 4-fluorobenzoyl chloride by following the procedure A (step 1). The intermediate product from the step 1 was aminated using monomethylamine to yield the final product. Starting from (1000 mg, 4.12 mmol) of 6-nitro-2,4-dichloroquinazoline, 400 mg (Yield, 22%) of the final product was isolated. MS (ESI) m/z 461.3.

### Example 188

### Preparation of N-[4-({[6-nitro-2-(methylamino)quinazolin-4-yl]amino}methyl)phenyl]nicotinamide

N-[4-({[6-nitro-2-(methylamino)quinazolin-4-yl]amino}methyl)phenyl]nicotinamide was prepared starting from 6-nitro-2,4-dichloroquinazoline, 4-aminobenzylamine and nicotinoylchloride by following the procedure A (step 1). The intermediate product from the step 1 was aminated using monomethylamine to yield the final product. Starting from (2 g, 8.23 mmol) of 6-nitro-2,4-dichloroquinazoline, (630 mg, Yield, 18%) of the final product was isolated. MS (ESI) m/z 400.1. mp 98-106° C.

### Example 189

### Preparation of 3,4-difluoro-N-[4-({[5-methyl-2-(methylamino)quinazolin-4-yl]amino}methyl)phenyl]benzamide

3,4-difluoro-N-[4-({[5-methyl-2-(methylamino)quinazolin-4-yl]amino}methyl)phenyl]benzamide was prepared starting from 5-methyl-2,4-dichloroquinazoline, 4-aminobenzylamine and 3,4-difluorobenzoylchloride by following the procedure A (step 1). The intermediate product from the step 1 was aminated using monomethylamine to yield the final product. Starting from (1000 mg, 4.69 mmol) of 5-methyl-2,4-dichloroquinazoline, 20 mg (Yield, 1%) of the final product was isolated. MS (ESI) m/z 434.3.

### Example 190

### Preparation of 4-fluoro-N-[4-({[8-methyl-2-(methylamino)quinazolin-4-yl]amino}methyl)phenyl]benzamide

4-fluoro-N-[4-({[8-methyl-2-(methylamino)quinazolin-4-yl]amino}methyl)phenyl]benzamide was prepared starting from 8-methyl-2,4-dichloroquinazoline, 4-aminobenzylamine and 4-fluorobenzoylchloride by following the procedure A (step 1). The intermediate product from the step 1 was aminated using monomethylamine to yield the final product. Starting from (500 mg, 2.35 mmol) of 8-methyl-2,4-dichloroquinazoline, 274 mg (Yield, 28%) of the final product was isolated. MS (ESI) m/z 416.1.

### Example 191

### Preparation of 4-chloro-N-[4-({[8-methyl-2-(methylamino)quinazolin-4-yl]amino}methyl)phenyl]benzamide

4-chloro-N-[4-({[8-methyl-2-(methylamino)quinazolin-4-yl]amino}methyl)phenyl]benzamide was prepared starting from 8-methyl-2,4-dichloroquinazoline, 4-aminobenzylamine and 4-chlorobenzoylchloride by following the procedure A (step 1). The intermediate product from the step 1 was aminated using monomethylamine to yield the final product. Starting from (500 mg, 2.35 mmol) of 8-methyl-2,4-dichloroquinazoline, 320 mg (Yield, 32%) of the final product was isolated. MS (ESI) m/z 432.1.

### Example 192

### Preparation of 4-fluoro-N-[4-({[5-methyl-2-(methylamino)quinazolin-4-yl]amino}methyl)phenyl]benzamide

4-fluoro-N-[4-({[5-methyl-2-(methylamino)quinazolin-4-yl]amino}methyl)phenyl]benzamide was prepared starting from 5-methyl-2,4-dichloroquinazoline (426 mg, 2 mmol), 4-aminobenzylamine and 4-fluorobenzoylchloride by following the procedure A (step 1) to give the 4-fluoro-N-[4-({[2-chloro-5-methyl-quinazolin-4-yl]amino}methyl)phenyl]benzamide (490 mg, yield 58%). The product (250 mg, 0.6 mmol) from the step 1 was aminated with monomethylamine to obtain the final product (98 mg, yield, 40%). MS (ESI) m/z 416.3; mp 228-230° C.

### Example 193

### Preparation of 4-chloro-N-[4-({[5-methyl-2-(methylamino)quinazolin-4-yl]amino}methyl)phenyl]benzamide

4-chloro-N-[4-({[5-methyl-2-(methylamino)quinazolin-4-yl]amino}methyl)phenyl]benzamide was prepared starting from 5-methyl-2,4-dichloroquinazoline (426 mg, 2 mmol), 4-aminobenzylamine and 4-chlorobenzoylchloride by following the procedure A (step 1) to give the 4-chloro-N-[4-({[2-chloro-5-methyl-quinazolin-4-yl]amino}methyl)phenyl]benzamide (155 mg, yield 18%). The product (120 mg, 0.28 mmol) from the step 1 was aminated with monomethylamine to obtain the final product (71 mg, yield, 60%). MS (ESI) m/z 432.3.

### Example 194

### Preparation of 6-chloro-N-[4-({[5-methyl-2-(methylamino)quinazolin-4-yl]amino}methyl)phenyl]nicotinamide

6-chloro-N-[4-({[5-methyl-2-(methylamino)quinazolin-4-yl]amino}methyl)phenyl]nicotinamide was prepared starting from 8-methyl-2,4-dichloroquinazoline (639 mg, 3.0 mmol), 4-aminobenzylamine and 6-chloronicotinoyl chloride by following the procedure A (step 1) to give the 6-chloro-N-[4-({[2-chloro-8-methyl-quinazolin-4-yl]amino}methyl)phenyl]nicotinamide (490 mg, yield 37%). The product (200 mg, 0.46 mmol) from the step 1 was aminated with monomethylamine hydrochloride to obtain the final product (66 mg, yield, 33%). MS (ESI) m/z 432.3; mp 233-236° C.

### Example 195

### Preparation of 4-chloro-N-[4-({[7-methyl-2-(methylamino)quinazolin-4-yl]amino}methyl)phenyl]benzamide

4-chloro-N-[4-({[7-methyl-2-(methylamino)quinazolin-4-yl]amino}methyl)phenyl]benzamide was prepared starting from 7-methyl-2,4-dichloroquinazoline (500 mg, 3.0 mmol), 4-aminobenzylamine and 4-chlorobenzoyl chloride by following the procedure A (step 1) to give the 4-chloro-N-[4-({[2-chloro-7-methyl-quinazolin-4-yl]amino}methyl)phenyl]benzamide (845 mg, yield 82%). The product (200 mg, 0.46 mmol) from the step 1 was aminated with monomethylamine hydrochloride to obtain the final product (150 mg, yield, 35%). MS (ESI) m/z 432.2 mp 264-266° C.

### Example 196

### Preparation of 4-chloro-N-[4-({[2-(dimethylamino)-7-methyl-quinazolin-4-yl]amino}methyl)phenyl]benzamide

4-chloro-N-[4-({[2-(dimethylamino)-7-methyl-quinazolin-4-yl]amino}methyl)phenyl]benzamide was prepared starting from 7-methyl-2,4-dichloroquinazoline (500 mg, 3.0 mmol), 4-aminobenzylamine and 4-chlorobenzoyl chloride by following the procedure A (step 1) to give the 4-chloro-N-[4-({[2-chloro-7-methyl-quinazolin-4-yl]amino}methyl)phenyl]benzamide (845 mg, yield 82%). The product (150 mg, 0.35 mmol) from the step 1 was aminated with dimethylamine to obtain the final product (82 mg, yield, 54%). MS (ESI) m/z 446.1.

### Example 197

### Preparation of 4-chloro-N-{4-[({7-methyl-2-[(2-pyridin-2-ylethyl)amino]quinazolin-4-yl}amino)methyl]phenyl}benzamide

4-chloro-N-{4-[({7-methyl-2-[(2-pyridin-2-ylethyl)amino]quinazolin-4-yl}amino)methyl]phenyl}benzamide was prepared starting from 7-methyl-2,4-dichloroquinazoline (500 mg, 3.0 mmol), 4-aminobenzylamine and 4-chlorobenzoyl chloride by following the procedure A (step 1) to give the 4-chloro-N-[4-({[2-chloro-7-methyl-quinazolin-4-yl]amino}methyl)phenyl]benzamide (845 mg, yield 82%). The product (150 mg, 0.35 mmol) from the step 1 was aminated with 2-ethylamino-2-pyridine to obtain the final product (95 mg, yield, 53%). MS (ESI) m/z 523.1.

### Example 198

### Preparation of N-(4-{[(2-azepan-1-yl-7-methylquinazolin-4-yl)amino]methyl}phenyl)-4-chlorobenzamide

N-(4-{[(2-azepan-1-yl-7-methylquinazolin-4-yl)amino]methyl}phenyl)-4-chlorobenzamide was prepared starting from 7-methyl-2,4-dichloroquinazoline (500 mg, 3.0 mmol), 4-aminobenzylamine and 4-chlorobenzoyl chloride by following the procedure A (step 1) to give the 4-chloro-N-[4-({[2-chloro-7-methyl-quinazolin-4-yl]amino}methyl)phenyl]benzamide (845 mg, yield 82%). The product (156 mg, 0.36 mmol) from the step 1 was aminated with aza-cycloheptane to obtain the final product (124 mg, yield, 70%). MS (ESI) m/z 500.3; mp 192-194° C.

### Procedure B (Step 1)

To a stirred suspension of N-[4-(aminomethyl)phenyl]-4-fluorobenzamide (1.40 g, 5.73 mmol) and triethylamine (2 mL) in THF (20 mL) at rt was added 7-methyl-2,4-dichloroquinazoline (1.22 g, 5.73 mmol) dissolved in CHCl₃ (10 mL) and the mixture was kept stirring for a minimum of 3 hours. After TLC showed the complete disappearance of the 7-methyl-2,4-dichloroquinazoline, CHCl₃ (250 mL) and water (25 mL) was added. The layers were separated, the aqueous layer was extracted twice with CHCl₃ (25 mL), and the combined organic layers were dried over MgSO₄. After removal of MgSO₄ by filtration and evaporation of solvents the crude product was purified by column chromatography with hexane/CH₂Cl₂/TEA to give N-(4-{[(2-chloro-7-methylquinazolin-4-yl)amino]methyl}phenyl)-4-fluorobenzamide (1.80 g, 73% yield). MS (ESI) m/z 421.2.

### Procedure B (Step 2)

N-(4-{[(2-chloro-7-methylquinazolin-4-yl)amino]methyl}phenyl)-4-fluorobenzamide (1 mmol) obtained by the procedure B, (step 1) was taken up either in a sealed tube or in round bottom flask and was suspended in and appropriate solvent THF (5 mL) or (dioxane, DMF, 2-propanol etc.) (5 mL). (If the reactant amine was mono methylamine or dimethylamine sealed tube was used and for other amines round bottom flask can be used.) The appropriate amine or amine hydrochloride was added and the mixture was heated under stirring over 2-16 h to 100-120° C. After the reaction was completed, the solvents were removed in vacuum and the crude compound was purified by preparative HPLC (high pressure liquid chromatography) using acetonitrile (ACN)/water/(NH₃ or TFA)-gradients as eluent or column chromatography with CH₂Cl₂/MeOH/NH₃ to give the diamino quinazolines in yields between 65-95%.

### Example 199

### Preparation of N-[4-({[2-(dimethylamino)-7-methylquinazolin-4-yl]amino}methyl)phenyl]-4-fluorobenzamide

N-[4-({[2-(dimethylamino)-7-methylquinazolin-4-yl]amino}methyl)phenyl]-4-fluorobenzamide was prepared by amination of N-(4-{[(2-chloro-7-methylquinazolin-4-yl)amino]methyl}phenyl)-4-fluorobenzamide (150 mg, 0.36 mmol) with 2M dimethylamine hydrochloride in 2-propanol following the procedure B (step 2). After purification by column chromatography and solvent removal the final product (110 mg, yield, 71%) was isolated. MS (ESI) m/z 430.3.

### Example 200

### Preparation of 4-fluoro-N-[4-({[7-methyl-2-(4-pyridin-2-ylpiperazin-1-yl)quinazolin-4-yl]amino}methyl)phenyl]benzamide

4-fluoro-N-[4-({[7-methyl-2-(4-pyridin-2-ylpiperazin-1-yl)quinazolin-4-yl]amino}methyl)phenyl]benzamide was prepared by amination of N-(4-{[(2-chloro-7-methylquinazolin-4-yl)amino]methyl}phenyl)-4-fluorobenzamide (42 mg, 0.1 mmol) with 1-(2-pyridyl)-piperazine hydrochloride and NEt₃ in dioxan following the procedure B (step 2). After purification by HPLC and solvent removal the final product (22 mg, yield, 65%) was isolated. MS (ESI) m/z 548.2.

### Example 201

### Preparation of N-{4-[({2-[[3-(dimethylamino)propyl](methyl)amino]-7-methylquinazolin-4-yl}amino)methyl]phenyl}-4-fluorobenzamide

N-{4-[({2-[[3-(dimethylamino)propyl](methyl)amino]-7-methylquinazolin-4-yl}amino)methyl]phenyl}-4-fluorobenzamide was prepared by amination of N-(4-{[(2-chloro-7-methylquinazolin-4-yl)amino]methyl}phenyl)-4-fluorobenzamide (42 mg, 0.1 mmol) with N,N,N′-trimethyl popyldiamine in dioxan following the procedure B (step 2). After purification by HPLC and solvent removal the final product (20 mg, yield, 66%) was isolated. MS (ESI) m/z 501.6.

### Example 202

### Preparation of N-(4-{[(2-azetidin-1-yl-7-methylquinazolin-4-yl)amino]methyl}phenyl)-4-fluorobenzamide

N-(4-{[(2-azetidin-1-yl-7-methylquinazolin-4-yl)amino]methyl}phenyl)-4-fluorobenzamide was prepared by amination of N-(4-{[(2-chloro-7-methylquinazolin-4-yl)amino]methyl}phenyl)-4-fluorobenzamide (42 mg, 0.1 mmol) with azetidine hydrochloride and NEt₃ in dioxan following the procedure B (step 2). After purification by HPLC and solvent removal the final product (20 mg, yield, 74%) was isolated. MS (ESI) m/z 442.2.

### Example 203

### Preparation of N-{4-[({2-[benzyl(methyl)amino]-7-methylquinazolin-4-yl}amino)methyl]phenyl}-4-fluorobenzamide

N-{4-[({2-[benzyl(methyl)amino]-7-methylquinazolin-4-yl}amino)methyl]phenyl}-4-fluorobenzamide was prepared by amination of N-(4-{[(2-chloro-7-methylquinazolin-4-yl)amino]methyl}phenyl)-4-fluorobenzamide (42 mg, 0.1 mmol) with N-benzylmethylamine in dioxan following the procedure B (step 2). After purification by HPLC and solvent removal the final product (17 mg, yield, 54%) was isolated. MS (ESI) m/z 506.6.

### Example 204

### Preparation of 4-fluoro-N-[4-({[7-methyl-2-(4-methylpiperazin-1-yl)quinazolin-4-yl]amino}methyl)phenyl]benzamide

4-fluoro-N-[4-({[7-methyl-2-(4-methylpiperazin-1-yl)quinazolin-4-yl]amino}methyl)phenyl]benzamide was prepared by amination of N-(4-{[(2-chloro-7-methylquinazolin-4-yl)amino]methyl}phenyl)-4-fluorobenzamide (42 mg, 0.1 mmol) 1-methyl piperazine in dioxan following the procedure B (step 2). After purification by HPLC and solvent removal the final product (20 mg, yield, 67%) was isolated. MS (ESI) m/z 485.6.

### Example 205

### Preparation of 4-fluoro-N-{4-[({2-[(2S)-2-(methoxymethyl)pyrrolidin-1-yl]-7-methylquinazolin-4-yl}amino)methyl]phenyl}benzamide

4-fluoro-N-{4-[({2-[(2S)-2-(methoxymethyl)pyrrolidin-1-yl]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (42 mg, 0.1 mmol) 2-(S)-methoxymethylpyrrolidine in dioxan following the procedure B (step 2). After purification by HPLC and solvent removal the final product (27 mg, yield, 89%) was isolated. MS (ESI) m/z 500.6.

### Example 206

### Preparation of WAC-572963 4-fluoro-N-(4-{[(7-methyl-2-piperidin-1-ylquinazolin-4-yl)amino]methyl}phenyl)benzamide

4-fluoro-N-(4-{[(7-methyl-2-piperidin-1-ylquinazolin-4-yl)amino]methyl}phenyl)benzamide was prepared by amination of N-(4-{[(2-chloro-7-methylquinazolin-4-yl)amino]methyl}phenyl)-4-fluorobenzamide (42 mg, 0.1 mmol) and piperidine in dioxan following the procedure B (step 2). After purification by HPLC and solvent removal the final product (11 mg, yield, 38%) was isolated. MS (ESI) m/z 470.6.

### Example 207

### Preparation of 4-fluoro-N-(4-{[(7-methyl-2-morpholin-4-ylquinazolin-4-yl)amino]methyl}phenyl)benzamide

4-fluoro-N-(4-{[(7-methyl-2-morpholin-4-ylquinazolin-4-yl)amino]methyl}phenyl)benzamide was prepared by amination of N-(4-{[(2-chloro-7-methylquinazolin-4-yl)amino]methyl}phenyl)-4-fluorobenzamide (42 mg, 0.1 mmol) morpholine in dioxan following the procedure B (step 2). After purification by HPLC and solvent removal the final product (17 mg, yield, 60%) was isolated. MS (ESI) m/z 472.5.

### Example 208

### Preparation of 4-fluoro-N-(4-{[(7-methyl-2-piperazin-1-ylquinazolin-4-yl)amino]methyl}phenyl)benzamide

4-fluoro-N-(4-{[(7-methyl-2-piperazin-1-ylquinazolin-4-yl)amino]methyl}phenyl)benzamide was prepared by amination of N-(4-{[(2-chloro-7-methylquinazolin-4-yl)amino]methyl}phenyl)-4-fluorobenzamide (42 mg, 0.1 mmol) piperazine in dioxan following the procedure B (step 2). After purification by HPLC and solvent removal the final product (21 mg, yield, 72%) was isolated. MS (ESI) m/z 471.6.

### Example 209

### Preparation of 4-fluoro-N-(4-{[(7-methyl-2-pyrrolidin-1-ylquinazolin-4-yl)amino]methyl}phenyl)benzamide

4-fluoro-N-(4-{[(7-methyl-2-pyrrolidin-1-ylquinazolin-4-yl)amino]methyl}phenyl)benzamide was prepared by amination of N-(4-{[(2-chloro-7-methylquinazolin-4-yl)amino]methyl}phenyl)-4-fluorobenzamide (42 mg, 0.1 mmol) pyrrolidine in dioxan following the procedure B (step 2). After purification by HPLC and solvent removal the final product (16 mg, yield, 56%) was isolated. MS (ESI) m/z 456.5.

### Example 210

### Preparation of N-{4-[({2-[ethyl(methyl)amino]-7-methylquinazolin-4-yl}amino)methyl]phenyl}-4-fluorobenzamide

N-{4-[({2-[ethyl(methyl)amino]-7-methylquinazolin-4-yl}amino)methyl]phenyl}-4-fluorobenzamide was prepared by amination of N-(4-{[(2-chloro-7-methylquinazolin-4-yl)amino]methyl}phenyl)-4-fluorobenzamide (42 mg, 0.1 mmol) N-ethylmethylamine in dioxan following the procedure B (step 2). After purification by HPLC and solvent removal the final product (23 mg, yield, 85%) was isolated. MS (ESI) m/z 444.5.

### Example 211

### Preparation of N-[4-({[2-(diethylamino)-7-methylquinazolin-4-yl]amino}methyl)phenyl]-4-fluorobenzamide

N-[4-({[2-(diethylamino)-7-methylquinazolin-4-yl]amino}methyl)phenyl]-4-fluorobenzamide was prepared by amination of N-(4-{[(2-chloro-7-methylquinazolin-4-yl)amino]methyl}phenyl)-4-fluorobenzamide (42 mg, 0.1 mmol) diethylamine in dioxan following the procedure B (step 2). After purification by HPLC and solvent removal the final product (18 mg, yield, 64%) was isolated. MS (ESI) m/z 458.6.

### Example 212

### Preparation of 4-fluoro-N-[4-({[7-methyl-2-(4-phenylpiperazin-1-yl)quinazolin-4-yl]amino}methyl)phenyl]benzamide

4-fluoro-N-[4-({[7-methyl-2-(4-phenylpiperazin-1-yl)quinazolin-4-yl]amino}methyl)phenyl]benzamide was prepared by amination of N-(4-{[(2-chloro-7-methylquinazolin-4-yl)amino]methyl}phenyl)-4-fluorobenzamide (30 mg, 0.07 mmol) 1-phenylpiperazine in dioxan following the procedure B (step 2). After purification by HPLC and solvent removal the final product (19 mg, yield, 40%) was isolated as bis-TFA salt. MS (ESI) m/z 547.7.

### Example 213

### Preparation of 4-fluoro-N-{4-[({7-methyl-2-[4-(2-oxo-2-pyrrolidin-1-ylethyl)piperazin-1-yl]quinazolin-4-yl}amino)methyl]phenyl}benzamide

4-fluoro-N-{4-[({7-methyl-2-[4-(2-oxo-2-pyrrolidin-1-ylethyl)piperazin 1-yl]quinazolin-4-yl}amino)methyl]phenyl}benzamide was prepared by amination of N-(4-{[(2-chloro-7-methylquinazolin-4-yl)amino]methyl}phenyl)-4-fluorobenzamide (30 mg, 0.07 mmol) (1-pyrrolidinecarbonylmethyl)piperazine in dioxan following the procedure B (step 2). After purification by HPLC and solvent removal the final product (38 mg, yield, 78%) was isolated as bis-TFA salt. MS (ESI) m/z 582.7.

### Example 214

### Preparation of 4-fluoro-N-{4-[({2-[(2-hydroxyethyl)(methyl)amino]-7-methylquinazolin-4-yl}amino)methyl]phenyl}benzamide

4-fluoro-N-{4-[({2-[(2-hydroxyethyl)(methyl)amino]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (30 mg, 0.07 mmol) and 2-methylaminoethanol in dioxan following the procedure B (step 2). After purification by HPLC and solvent removal the final product (22 mg, yield, 61%) was isolated as bis-TFA salt. MS (ESI) m/z 460.5.

### Example 215

### Preparation of N-{4-[({2-[4-(1,3-benzodioxol-5-ylmethyl)piperazin-1-yl]-7-methylquinazolin-4-yl}amino)methyl]phenyl}-4-fluorobenzamide

N-{4-[({2-[4-(1,3-benzodioxol-5-ylmethyl)piperazin-1-yl]-7-methylquinazolin-4-yl}amino)methyl]phenyl}-4-fluorobenzamide was prepared by amination of N-(4-{[(2-chloro-7-methylquinazolin-4-yl)amino]methyl}phenyl)-4-fluorobenzamide (30 mg, 0.07 mmol) and 1-piperonylpiperazine in dioxan following the procedure B (step 2). After purification by HPLC and solvent removal the final product (36 mg, yield, 71%) was isolated as bis-TFA salt. MS (ESI) m/z 605.7.

### Example 216

### Preparation of 4-fluoro-N-[4-({[7-methyl-2-(4-pyrimidin-2-ylpiperazin-1-yl)quinazolin-4-yl]amino}methyl)phenyl]benzamide

4-fluoro-N-[4-({[7-methyl-2-(4-pyrimidin-2-ylpiperazin-1-yl)quinazolin-4-yl]amino}methyl)phenyl]benzamide was prepared by amination of N-(4-{[(2-chloro-7-methylquinazolin-4-yl)amino]methyl}phenyl)-4-fluorobenzamide (30 mg, 0.07 mmol) and 1-(2-pyrimidyl)-piperazine hydrochloride and NEt₃ in dioxan following the procedure B (step 2). After purification by HPLC and solvent removal the final product (18 mg, yield, 39%) was isolated as bis-TFA salt. MS (ESI) m/z 549.6.

### Example 217

### Preparation of 4-fluoro-N-[4-({[2-(4-formylpiperazin-1-yl)-7-methylquinazolin-4-yl]amino}methyl)phenyl]benzamide

4-fluoro-N-[4-({[2-(4-formylpiperazin-1-yl)-7-methylquinazolin-4-yl]amino}methyl)phenyl]benzamide was prepared by amination of N-(4-{[(2-chloro-7-methylquinazolin-4-yl)amino]methyl}phenyl)-4-fluorobenzamide (30 mg, 0.07 mmol) and 1-piperazine-carboxaldehyde in dioxan following the procedure B (step 2). After purification by HPLC and solvent removal the final product (20 mg, yield, 52%) was isolated as bis-TFA salt. MS (ESI) m/z 499.6.

### Example 218

### Preparation of ethyl 4-[4-({4-[(4-fluorobenzoyl)amino]benzyl}amino)-7-methylquinazolin-2-yl]piperazine-1-carboxylate

ethyl 4-[4-({4-[(4-fluorobenzoyl)amino]benzyl}amino)-7-methylquinazolin-2-yl]piperazine-1-carboxylate was prepared by amination of N-(4-{[(2-chloro-7-methylquinazolin-4-yl)amino]methyl}phenyl)-4-fluorobenzamide (30 mg, 0.07 mmol) and 1-Ethyl-piperazine carboxylate in dioxan following the procedure B (step 2). After purification by HPLC and solvent removal the final product (23 mg, yield, 58%) was isolated as bis-TFA salt. MS (ESI) m/z 543.6.

### Example 219

### Preparation of 4-fluoro-N-(4-{[(2-{4-[2-(isopropylamino)-2-oxoethyl]piperazin-1-yl}-7-methylquinazolin-4-yl)amino]methyl}phenylbenzamide

4-fluoro-N-(4-{[(2-{4-[2-(isopropylamino)-2-oxoethyl]piperazin-1-yl}-7-methylquinazolin-4-yl)amino]methyl}phenyl)benzamide was prepared by amination of N-(4-{[(2-chloro-7-methylquinazolin-4-yl)amino]methyl}phenyl)-4-fluorobenzamide (30 mg, 0.07 mmol) and N-isopropyl-piperazine acetamide in dioxan following the procedure B (step 2). After purification by HPLC and solvent removal the final product (32 mg, yield, 76%) was isolated as bis-TFA salt. MS (ESI) m/z 570.7.

### Example 220

### Preparation of 4-fluoro-N-{4-[({2-[(2-methoxyethyl)(methyl)amino]-7-methylquinazolin-4-yl}amino)methyl]phenyl}benzamide

4-fluoro-N-{4-[({2-[(2-methoxyethyl)(methyl)amino]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (30 mg, 0.07 mmol) and 2-methoxyethyl methylamine in dioxan following the procedure B (step 2). After purification by HPLC and solvent removal the final product (10 mg, yield, 29%) was isolated as bis-TFA salt. MS (ESI) m/z 474.6.

### Example 221

### Preparation of 4-fluoro-N-{4-[({2-[(2-furylmethyl)(methyl)amino]-7-methylquinazolin-4-yl}amino)methyl]phenyl}benzamide

4-fluoro-N-{4-[({2-[(2-furylmethyl)(methyl)amino]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (30 mg, 0.07 mmol) and methylfurfurylamine in dioxan following the procedure B (step 2). After purification by HPLC and solvent removal the final product (23 mg, yield, 62%) was isolated as bis-TFA salt. MS (ESI) m/z 496.6.

### Example 222

### Preparation of 4-fluoro-N-{4-[({7-methyl-2-[methyl(2-pyridin-2-ylethyl)amino]quinazolin-4-yl}amino)methyl]phenyl}benzamide

4-fluoro-N-{4-[({7-methyl-2-[methyl(2-pyridin-2-ylethyl)amino]quinazolin-4-yl}amino)methyl]phenyl}benzamide was prepared by amination of N-(4-{[(2-chloro-7-methylquinazolin-4-yl)amino]methyl}phenyl)-4-fluorobenzamide (30 mg, 0.07 mmol) and 2-(2-methylaminoethyl)pyridine in dioxan following the procedure B (step 2). After purification by HPLC and solvent removal the final product (33 mg, yield, 86%) was isolated as bis-TFA salt. MS (ESI) m/z 521.6.

### Example 223

### Preparation of N-[4-({[2-(4-acetyl-1,4-diazepan-1-yl)-7-methylquinazolin-4-yl]amino}methyl)phenyl]-4-fluorobenzamide

N-[4-({[2-(4-acetyl-1,4-diazepan-1-yl)-7-methylquinazolin-4-yl]amino}methyl)phenyl]-4-fluorobenzamide was prepared by amination of N-(4-{[(2-chloro-7-methylquinazolin-4-yl)amino]methyl}phenyl)-4-fluorobenzamide (30 mg, 0.07 mmol) and N-acetyl homopiperazine in dioxan following the procedure B (step 2). After purification by HPLC and solvent removal the final product (29 mg, yield, 73%) was isolated as bis-TFA salt. MS (ESI) m/z 527.6.

### Example 224

### Preparation of 4-fluoro-N-{4-[({2-[2-(2-hydroxyethyl)piperidin-1-yl]-7-methylquinazolin-4-yl}amino)methyl]phenyl}benzamide

N-{4-[({2-[2-(2-hydroxyethyl)piperidin-1-yl]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (25 mg, 0.06 mmol) and 2-(2-hydroxyethyl)piperidine in dioxan following the procedure B (step 2). After purification by HPLC and solvent removal the final product (21 mg, yield, 65%) was isolated. MS (ESI) m/z 514.6.

### Example 225

### Preparation of 4-fluoro-N-{4-[({7-methyl-2-[(3R)-3-methylpiperazin-1-yl]quinazolin-4-yl}amino)methyl]phenyl}benzamide

N-{4-[({2-[2-(2-hydroxyethyl)piperidin-1-yl]-7-methylquinazolin-4-yl}amino)methyl]phenyl}benzamide was prepared by amination of N-(4-{[(2-chloro-7-methylquinazolin-4-yl)amino]methyl}phenyl)-4-fluorobenzamide (25 mg, 0.06 mmol) and 2-(R)-methyl piperazine in dioxan following the procedure B (step 2). After purification by HPLC and solvent removal the final product (19 mg, yield, 64%) was isolated. MS (ESI) m/z 485.6.

### Example 226

### Preparation of 4-fluoro-N-[4-({[7-methyl-2-(4-pyrrolidin-1-ylpiperidin-1-yl)quinazolin-4-yl]amino}methyl)phenyl]benzamide

4-fluoro-N-[4-({[7-methyl-2-(4-pyrrolidin-1-ylpiperidin-1-yl)quinazolin-4-yl]amino}methyl)phenyl]benzamide was prepared by amination of N-(4-{[(2-chloro-7-methylquinazolin-4-yl)amino]methyl}phenyl)-4-fluorobenzamide (25 mg, 0.06 mmol) and 4-(1-pyrrolidinyl)-piperidine in dioxan following the procedure B (step 2). After purification by HPLC and solvent removal the final product (18 mg, yield, 56%) was isolated. MS (ESI) m/z 539.7.

### Example 227

### Preparation of N-[4-({[2-(4-ethylpiperazin-1-yl)-7-methylquinazolin-4-yl]amino}methyl)phenyl]-4-fluorobenzamide

N-[4-({[2-(4-ethylpiperazin-1-yl)-7-methylquinazolin-4-yl]amino}methyl)phenyl]-4-fluorobenzamide was prepared by amination of N-(4-{[(2-chloro-7-methylquinazolin-4-yl)amino]methyl}phenyl)-4-fluorobenzamide (25 mg, 0.06 mmol) and 1-ethypiperazine in dioxan following the procedure B (step 2). After purification by HPLC and solvent removal the final product (28 mg, yield, 93%) was isolated. MS (ESI) m/z 499.6.

### Example 228

### Preparation of 4-fluoro-N-{4-[({7-methyl-2-[(2S)-2-(pyrrolidin-1-ylmethyl)pyrrolidin-1-yl]quinazolin-4-yl}amino)methyl]phenyl}benzamide

4-fluoro-N-{4-[({7-methyl-2-[(2S)-2-(pyrrolidin-1-ylmethyl)pyrrolidin-1-yl]quinazolin-4-yl}amino)methyl]phenyl}benzamide was prepared by amination of N-(4-{[(2-chloro-7-methylquinazolin-4-yl)amino]methyl}phenyl)-4-fluorobenzamide (25 mg, 0.06 mmol) and S-2-(1-pyrrolidinylmethyl)-pyrrolidine in dioxan following the procedure B (step 2). After purification by HPLC and solvent removal the final product (14 mg, yield, 43%) was isolated. MS (ESI) m/z 539.7.

### Example 229

### Preparation of 4-fluoro-N-(4-{[(7-methyl-2-{[3-(4-methylpiperazin-1-yl)propyl]amino}quinazolin-4-yl)amino]methyl}phenyl)benzamide

N-(4-{[(7-methyl-2-{[3-(4-methylpiperazin-1-yl)propyl]amino}quinazolin-4-yl)amino]methyl}phenyl)benzamide was prepared by amination of N-(4-{[(2-chloro-7-methylquinazolin-4-yl)amino]methyl}phenyl)-4-fluorobenzamide (25 mg, 0.06 mmol) and 1-(3-aminopropyl)-4-methylpiperazine in dioxan following the procedure B (step 2). After purification by HPLC and solvent removal the final product (32 mg, yield, 95%) was isolated. MS (ESI) m/z 542.7.

### Example 230

### Preparation of 4-fluoro-N-[4-({[7-methyl-2-(methylamino)quinazolin-4-yl]amino}methyl)phenyl]benzamide

4-fluoro-N-[4-({[7-methyl-2-(methylamino)quinazolin-4-yl]amino}methyl)phenyl]benzamide was prepared by amination of N-(4-{[(2-chloro-7-methylquinazolin-4-yl)amino]methyl}phenyl)-4-fluorobenzamide (250 mg, 0.6 mmol) and 2 M methylamine in THF following the procedure B (step 2). After purification by HPLC and solvent removal the final product (150 mg, yield, 76%) was isolated. MS (ESI) m/z 416.2.

### Example 231

### Preparation of 4-fluoro-N-[4-({[7-methyl-2-(propylamino)quinazolin-4-yl]amino}methyl)phenyl]benzamide

4-fluoro-N-[4-({[7-methyl-2-(propylamino)quinazolin-4-yl]amino}methyl)phenyl]benzamide was prepared by amination of N-(4-{[(2-chloro-7-methylquinazolin-4-yl)amino]methyl}phenyl)-4-fluorobenzamide (150 mg, 0.36 mmol) and propylamine in THF following the procedure B (step 2). After purification by HPLC and solvent removal the final product (150 mg, yield, 76%) was isolated. MS (ESI) m/z 444.1; mp 170-174° C.

### Example 232

### Preparation of 4-fluoro-N-{4-[({7-methyl-2-[(2-pyridin-2-ylethyl)amino]quinazolin-4-yl}amino)methyl]phenyl}benzamide

4-fluoro-N-{4-[({7-methyl-2-[(2-pyridin-2-ylethyl)amino]quinazolin-4-yl}amino)methyl]phenyl}benzamide was prepared by amination of N-(4-{[(2-chloro-7-methylquinazolin-4-yl)amino]methyl}phenyl)-4-fluorobenzamide (150 mg, 0.36 mmol) and 2-ethylamino-2-pyridine in THF following the procedure B (step 2). After purification by HPLC and solvent removal the final product (63 mg, yield, 35%) was isolated. MS (ESI) m/z 507.2; mp 112-115° C.

### Example 233

### Preparation of N-{4-[({2-[(1-benzylpiperidin-4-yl)amino]-7-methylquinazolin-4-yl}amino)methyl]phenyl}-4-fluorobenzamide

N-{4-[({2-[(1-benzylpiperidin-4-yl)amino]-7-methylquinazolin-4-yl}amino)methyl]phenyl}-4-fluorobenzamide was prepared by amination of N-(4-{[(2-chloro-7-methylquinazolin-4-yl)amino]methyl}phenyl)-4-fluorobenzamide (150 mg, 0.36 mmol) and 4-amino-1-benzylpiperindine in THF following the procedure B (step 2). After purification by HPLC and solvent removal the final product (70 mg, yield, 34%) was isolated. MS (ESI) m/z 575.2; mp 135-138° C.

### Example 234

### Preparation of 4-fluoro-N-{4-[({7-methyl-2-[(3S)-3-methylpiperazin-1-yl]quinazolin-4-yl}amino)methyl]phenyl}benzamide

4-fluoro-N-{4-[({7-methyl-2-[(3S)-3-methylpiperazin-1-yl]quinazolin-4-yl}amino)methyl]phenyl}benzamide was prepared by amination of N-(4-{[(2-chloro-7-methylquinazolin-4-yl)amino]methyl}phenyl)-4-fluorobenzamide (150 mg, 0.36 mmol) and 4-amino-1-benzylpiperindine in THF following the procedure B (step 2). After purification by HPLC and solvent removal the final product (68 mg, yield, 39%) was isolated. MS (ESI) m/z 485; mp 200-202° C.

### Example 235

### Preparation of 1N-(4-{[(2-azepan-1-yl-7-methylquinazolin-4-yl)amino]methyl}phenyl)-4-fluorobenzamide

1N-(4-{[(2-azepan-1-yl-7-methylquinazolin-4-yl)amino]methyl}phenyl)-4-fluorobenzamide was prepared by amination of N-(4-{[(2-chloro-7-methylquinazolin-4-yl)amino]methyl}phenyl)-4-fluorobenzamide (150 mg, 0.36 mmol) and aza-cycloheptane in THF following the procedure B (step 2). After purification by HPLC and solvent removal the final product (132 mg, yield, 77%) was isolated. MS (ESI) m/z 484.3; mp 135-139° C.

### Example 236

### Preparation of N-[4-({[2-(3,3-dimethylpiperazin-1-yl)-7-methylquinazolin-4-yl]amino}methyl)phenyl]-4-fluorobenzamide

N-[4-({[2-(3,3-dimethylpiperazin-1-yl)-7-methylquinazolin-4-yl]amino}methyl)phenyl]-4-fluorobenzamide was prepared by amination of N-(4-{[(2-chloro-7-methylquinazolin-4-yl)amino]methyl}phenyl)-4-fluorobenzamide (150 mg, 0.36 mmol) and 2,2-dimethylpiperazine in THF following the procedure B (step 2). After purification by HPLC and solvent removal the final product (105 mg, yield, 59%) was isolated. MS (ESI) m/z 499.3.

### Example 237

### Preparation of 4-fluoro-N-{4-[({7-methyl-2-[(2-pyrrolidin-1-ylethyl)amino]quinazolin-4-yl}amino)methyl]phenyl}benzamide

4-fluoro-N-{4-[({7-methyl-2-[(2-pyrrolidin-1-ylethyl)amino]quinazolin-4-yl}amino)methyl]phenyl)}benzamide was prepared by amination of N-(4-{[(2-chloro-7-methylquinazolin-4-yl)amino]methyl}phenyl)-4-fluorobenzamide (150 mg, 0.36 mmol) and 2-ethylamino pyrrolidine in THF following the procedure B (step 2). After purification by HPLC and solvent removal the final product (55 mg, yield, 31%) was isolated. MS (ESI) m/z 499.1.

### Example 238

### Preparation of 4-bromo-N-[4-({[7-methyl-2-(methylamino)quinazolin-4-yl]amino}methyl)phenyl]benzamide

To a stirred suspension N-[4-(aminomethyl)phenyl]-4-bromobenzamide (590 mg, 1.41 mmol) and triethylamine (0.983 mL) in THF (6 mL) was added 7-methyl-2,4-dichloroquinazoline (300 mg, 1.41 mmol) at rt then the mixture was stirred overnight. Added CHCl₃ (50 mL) and water (10 mL), collected organic layer and the solvent was removed then triturated with CH2Cl2 to give 4-bromo-N-(4-((2-chloro-7-methylquinazolin-4-ylamino)methyl)phenyl)benzamide as a white solid (670 mg, yield, 99%). MS (ESI) m/z 481.1.

To the 4-bromo-N-(4-((2-chloro-7-methylquinazolin-4-ylamino)methyl)phenyl)benzamide (150 mg, 0.311 mmol), methylamine.HCl (462 mg, 6.842 mmol) and 2-propanol (3 mL) was heated under reflux overnight. The solvent was removed and purified by combi flash chromatography using 10% MeOH in CH₂Cl₂/EtOAc (1:1) to give the product (65 mg, yield, 44%) mp 295-298° C.; MS (ESI) m/z 476.1; HRMS: calcd for C24H22BrN5O+H+, 476.10805; found (ESI-FTMS, [M+H]1+), 476.10943.

### Example 239

### Preparation of 4-bromo-N-[4-({[2-(dimethylamino)-7-methylquinazolin-4-yl]amino}methyl)phenyl]benzamide

4-bromo-N-(4-((2-chloro-7-methylquinazolin-4-ylamino)methyl)phenyl)benzamide (150 mg, 0.311 mmol), dimethylamine (40% aqueous solution) (0.867 mL, 6.842 mmol) and THF (1 mL) was heated to 100° C. in a sealed tube overnight. The solvent was removed and purified by combi flash chromatography using 10% MeOH in CH₂Cl₂/EtOAc (1:1) to give the product (102 mg, yield, 67%). MS (ESI) m/z 490.2; HRMS: calcd for C25H24BrN5O+H+, 490.12370; found (ESI-FTMS, [M+H]1+), 490.12271.

### Example 240

### Preparation of 6-methyl-N-[4-({[7-methyl-2-(methylamino)quinazolin-4-yl]amino}methyl)phenyl]nicotinamide

To a stirred suspension N-[4-(aminomethyl)phenyl]-6-methylnicotinamide (440 mg, 0.939 mmole) and triethylamine (0.654 mL) in THF (4 mL) was added 7-methyl-2,4-dichloroquinazoline (200 mg, 0.939 mmol) at rt then the mixture was stirred overnight. Added CHCl₃ (50 mL) and water (10 mL), collected organic layer and the solvent was removed then triturated with CH₂Cl₂ to give N-(4-{[(2-chloro-7-methylquinazolin-4-yl)amino]methyl}phenyl)-6-methylnicotinamide as a white solid (420 mg, yield, 100%). MS (ESI) m/z 418.2.

To the N-(4-{[(2-chloro-7-methylquinazolin-4-yl)amino]methyl}phenyl)-6-methylnicotinamide (180 mg, 0.431 mmol), methylamine.HCl (631 mg, 9.34 mmol) and 2-propanol (6 mL) was heated under reflux overnight. The solvent was removed and purified by combi flash chromatography using 10% MeOH in CH₂Cl₂/EtOAc (1:1) to give the product (75 mg, yield, 42%) mp 257-259° C.; MS (ESI) m/z 413.2; MS (ESI) m/z 207.1; MS (ESI) m/z 246.1; HRMS: calcd for C24H24N6O+H+, 413.20843; found (ESI-FTMS, [M+H]1+), 413.20848.

### Example 241

### Preparation of N-[4-({[2-(dimethylamino)-7-methylquinazolin-4-yl]amino}methyl)phenyl]-6-methylnicotinamide

To the N-(4-{[(2-chloro-7-methylquinazolin-4-yl)amino]methyl}phenyl)-6-methylnicotinamide (180 mg, 0.431 mmol), dimethylamine (40% aqueous solution) (1.1 mL, 8.62 mmol) and THF (2 mL) was heated to 100° C. in a sealed tube overnight. The solvent was removed and purified by combi flash chromatography using 10% MeOH in CH₂Cl₂/EtOAc (1:1) to give the product (68 mg, yield, 37%). MS (ESI) m/z 427.3; MS (ESI) m/z 214.1; MS (ESI) m/z 234.7; HRMS: calcd for C25H26N6O+H+, 427.22408; found (ESI-FTMS, [M+H]1+), 427.22561.

### Example 242

### Preparation of 6-chloro-N-[4-({[2-(dimethylamino)-7-methylquinazolin-4-yl]amino}methyl)phenyl]nicotinamide

To the 6-chloro-N-(4-{[(2-chloro-7-methylquinazolin-4-yl)amino]methyl}phenyl)nicotinamide 4.89 grams, 11.16 mmol), dimethylamine.HCl (10.92 grams, 133.88 mmol) and DMF (30 mL) was heated to 100° C. for 1.5 hrs. Concentrated and added water the solid was collected and triturated with methanol to give the product (4.05 grams, yield 75%) as a mono hydrochloride salt. mp 325-329° C.; MS (ESI) m/z 447.2.

### Procedure D (Step 1):

To a stirred solution of 4-aminobenzylamine (285 mg, 2.33 mmol) and triethylamine (1200 mg, 11.7 mmol, 5 eq) in CHCl₃ (5 mL) at rt was added 7-methyl-2,4-dichloroquinazoline (500 mg, 2.33 mmol) as solid and the mixture was allowed to stir for a minimum of 3 hours. After TLC showed only minimal amounts of starting materials remaining, THF (10 mL), HOBT (629 mg, 4.66 mmol, 2 eq), N—BOC-isonipecotic acid (600 mg, 2.62 mmol, 1.1 eq) and EDCI (670 mg, 3.5 mmol, 1.5 eq) were added in the described order and the mixture was allowed to stir overnight. For work up CHCl₃ (100 mL) and water (20 mL) were added and the organic layer was separated. The aqueous layer was washed twice with CHCl₃ (20 mL) and the combined organic layers were dried over MgSO₄. After filtration of MgSO₄ and solvent removal, the 1.5 g crude product (which was a 1:1-mixture of (4-{4-[(2-Chloro-7-methyl-quinazolin-4-ylamino)-methyl]-phenylcarbamoyl}-piperidine-1-carboxylic acid tert-butyl ester) and 4-(tert-Butoxycarbonyl-{4-[(2-chloro-7-methyl-quinazolin-4-ylamino)-methyl]-phenyl}-aminocarbonyl)-piperidine-1-carboxylic acid tert-butyl ester) was taken forward without further purification.

### Procedure D (step 2): Preparation of tert-butyl 4-({[4-({[2-(dimethylamino)-7-methylquinazolin-4-yl]amino}methyl)phenyl]amino}carbonyl)piperidine-1-carboxylate

The crude product (1.5 g, 2.33 mmol) from procedure D (step 1) was placed in a round bottom flask with dimethylamine hydrochloride (4 g) and was dissolved in DMF (10 mL). The mixture was heated under stirring over 1-2 h to 120° C. After the reaction was completed, aqueous 1N NaOH (50 mL) and CHCl₃ (100 mL) was added; the layers were separated and the aqueous layer was washed twice with CHCl₃ (25 mL). Vacuum distillation of the combined organic layers led to a thick oil, which was stirred with water (30 mL) to form a solid. The solid was collected by filtration and dried at (50° C./14 mbar) to obtain 720 mg (yield, 59%) of tert-butyl 4-({[4-({[2-(dimethylamino)-7-methylquinazolin-4-yl]amino}methyl)phenyl]amino}carbonyl)piperidine-1-carboxylate. MS (ESI) e/z=521.

### Procedure D (step 3): Preparation of N-[4-({[2-(dimethylamino)-7-methylquinazolin-4-yl]amino}methyl)phenyl]piperidine-4-carboxamide

In a round bottom flask was added to a stirred suspension, of tert-butyl 4-({[4-({[2-(dimethylamino)-7-methylquinazolin-4-yl]amino}methyl)phenyl]amino}carbonyl)piperidine-1-carboxylate (600 mg, 1.15 mmol) in chloroform (15 mL), trifluoroacetic acid (5 mL). The mixture was stirred at room temperature for two hours until deprotection was completed. Solvents were removed in vacuo and the residue was taken up 5 mL ACN. When the solution was basified with 5N NaOH, precipitation of a solid was observed. The precipitate was collected by filtration washed with water (1 mL). After drying over night at 50° C./14 mbar, 280 mg (yield, 66%) of the product as off-white solid was obtained. MS (ESI) m/z 421.

### Procedure D (step 4): N-alkylation (or N-benzylation) of N-[4-({[2-(dimethylamino)-7-methylquinazolin-4-yl]amino}methyl)phenyl]piperidine-4-carboxamide

In a round bottom flask were dissolved N-[4-({[2-(dimethylamino)-7-methylquinazolin-4-yl]amino}methyl)phenyl]piperidine-4-carboxamide (280 mg, 0.66 mmol) and the appropriate aldehyde or ketone (1.33 mmol, 2 eq) in methanol (3 mL). A suspension of NaBH₃CN (84 mg, 1.33 mmol, 2 eq) and ZnCl₂ (90 mg, 0.66 mmol, 1 eq) in methanol (1 mL) was added slowly. The reaction was stirred for 2-6 h; the solvents were removed under reduced pressure and 1N NaOH, (2 mL); and THF/ethyl acetate (1:1) (10 mL) were added. The organic layer was separated and the aqueous layer was washed twice with 5 mL (1:1) THF/ethyl acetate. The combined organic layers were dried over MgSO₄. After removal of the drying agent by filtration, the solvents were distilled on a rota-evaporator and the crude compound was purified by preparative HPLC (high pressure liquid chromatography) using ACN/water/NH₃-gradients as eluent or column chromatography with CH₂Cl₂/MeOH/NH₃ to give the N-alkyl piperidine analogs in yields between 65-83%.

Some of the products were converted to the bis-HCl salts by dissolving in MeOH and addition of 4M HCl in dioxan (0.5 mL). The bis-HCl salt was obtained after solvent removal in vacuo.

### Example 243

### Preparation of N-[4-({[2-(dimethylamino)-7-methylquinazolin-4-yl]amino}methyl)phenyl]-1-methylpiperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using 37% aq formaldehyde solution to give product (15 mg, yield, 56%). MS (ESI) m/z 433.3.

### Example 244

### Preparation of N-[4-({[2-(dimethylamino)-7-methylquinazolin-4-yl]amino}methyl)phenyl]-1-isobutylpiperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using isobutyraldehyde to give product (18 mg, yield, 61%) MS (ESI) m/z 475.3.

### Example 245

### Preparation of 1-cyclohexyl-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using cyclohexanon to give product (7 mg, yield, 23%) MS (ESI) m/z 501.3.

### Example 246

### Preparation of N-[4-({[2-(dimethylamino)-7-methylquinazolin-4-yl]amino}methyl)phenyl]-1-(2-furylmethyl)piperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using furfural to give product (20 mg, yield, 65%). MS (ESI) m/z 499.

### Example 247

### Preparation of N-[4-({[2-(dimethylamino)-7-methylquinazolin-4-yl]amino}methyl)phenyl]-1-(4-methylbenzyl)piperidine-4-carboxamide

N-[4-({[2-(dimethylamino)-7-methylquinazolin-4-yl]amino}methyl)phenyl]piperidine-4-carboxamide (230 mg, 0.55 mmol) was N-benzylated according to procedure D (step 4) using p-toluoylaldehyde to give product (114 mg, yield, 35%) as bis hydrochloride salt. MS (ESI) m/z 523.

### Example 248

### Preparation of N-[4-({[2-(dimethylamino)-7-methylquinazolin-4-yl]amino}methyl)phenyl]-1-(1H-imidazol-2-ylmethyl)piperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using 2-imidazol-carbaldehyde to give product (18 mg, yield, 60%). MS (ESI) m/z 499.

### Example 249

### Preparation of 1-butyl-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (25 mg, 0.06 mmol) was N-alkylated according to procedure D (step 4) using butanal to give product (18 mg, yield, 62%). MS (ESI) m/z 475.

### Example 250

### Preparation of N-[4-({[2-(dimethylamino)-7-methylquinazolin-4-yl]amino}methyl)phenyl]-1-(4-methoxybenzyl)piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using p-anisaldehyde to give product (68 mg, yield, 23%) as bis hydrochloride salt. MS (ESI) m/z 539.4.

### Example 251

### Preparation of N-[4-({[2-(dimethylamino)-7-methylquinazolin-4-yl]amino}methyl)phenyl]-1-(4-fluorobenzyl)piperidine-4-carboxamide

N-[4-({[2-(dimethylamino)-7-methylquinazolin-4-yl]amino}methyl)phenyl]piperidine-4-carboxamide (280 mg, 0.66 mmol) was N-benzylated according to procedure D (step 4) using 4-fluorobenzaldehyde to give product (219 mg, yield, 55%) as bis hydrochloride salt. MS (ESI) m/z 527.4.

### Example 252

### Preparation of N-[4-({[2-(dimethylamino)-7-methylquinazolin-4-yl]amino}methyl)phenyl]-1-(2-fluorobenzyl)piperidine-4-carboxamide

N-[4-({[2-(dimethylamino)-7-methylquinazolin-4-yl]amino}methyl)phenyl]piperidine-4-carboxamide (230 mg, 0.55 mmol) was N-benzylated according to procedure D (step 4) using 2-fluorobenzaldehyde to give product (122 mg, yield, 37%) as bis hydrochloride salt. MS (ESI) m/z 527.4.

### Example 253

### Preparation of 1-(4-chlorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (230 mg, 0.55 mmol) was N-benzylated according to procedure D (step 4) using 4-chlorobenzaldehyde to give product (110 mg, yield, 32%) as bis hydrochloride salt. MS (ESI) m/z 543.4.

### Example 254

### Preparation of 1-(2,4-difluorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 2,4-difluorobenzaldehyde to give product (60 mg, yield, 23%). MS (ESI) m/z 545.5.

### Example 255

### Preparation of 1-(3,4-difluorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 3,4-difluorobenzaldehyde to give product (108 mg, yield, 41%). m.p.: 97-99° C. MS (ESI) m/z 545.5.

### Example 256

### Preparation of N-[4-({[2-(dimethylamino)-7-methylquinazolin-4-yl]amino}methyl)phenyl]-1-[4-(trifluoromethyl)benzyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 4-trifluoromethylbenzaldehyde to give product (88 mg, yield, 32%). m.p. 126-128° C. MS (ESI) m/z 577.5.

### Example 257

### Preparation of N-[4-({[2-(dimethylamino)-7-methylquinazolin-4-yl]amino}methyl)phenyl]-1-(pyridin-4-ylmethyl)piperidine-4-carboxamide

N-[4-({[2-(dimethylamino)-7-methylquinazolin-4-yl]amino}methyl)phenyl]piperidine-4-carboxamide (200 mg, 0.48 mmol) was N-alkylated according to procedure D (step 4) using 4-pyridinecarboxaldehyde and subsequently converted to the bis-HCl salt following procedure M to give the product (102 mg, yield, 42%). HRMS: calcd for C30H35N7O+H+, 510.29758; found (ESI-FTMS, [M+H]1+), 510.29608.

### Example 258

### Preparation of 1-(2-chloro-4-fluorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 2-chloro-4-fluorobenzaldehyde and subsequently converted to the bis-HCl salt to give the product (65 mg, yield, 24%) HRMS: calcd for C31H34ClFN6O+H+, 561.25394; found (ESI-FTMS, [M+H]1+), 561.25461.

### Example 259

### Preparation of 1-[(6-chloropyridin-3-yl)methyl]-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-alkylated according to procedure D (step 4) using 6-chloro-3-pyridinecarbaldehyde and subsequently converted to the bis-HCl salt to give the product (54 mg, yield, 18%) MS (ESI) m/z 544.4.

### Example 260

### Preparation of N-[4-({[2-(dimethylamino)-7-methylquinazolin-4-yl]amino}methyl)phenyl]-1-(2,4,6-trifluorobenzyl)piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 2,4,6-trifluorobenzaldehyde and subsequently converted to the bis-HCl salt to give the product (102 mg, yield, 34%). MS (ESI) m/z 563.4.

### Example 261

### Preparation of N-[4-({[2-(dimethylamino)-7-methylquinazolin-4-yl]amino}methyl)phenyl]-1-(3-fluorobenzyl)piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 3-fluorobenzaldehyde and subsequently converted to the bis-HCl salt to give the product (128 mg, yield, 45%). MS (ESI) m/z 527.4.

### Example 262

### Preparation of 1-(2,5-difluorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 2,5 difluorobenzaldehyde and subsequently converted to the bis-HCl salt to give the product (130 mg, yield, 44%). MS (ESI) m/z 545.3.

### Example 263

### Preparation of 1-(4-chloro-3-fluorobenzyl)-N-[4-({[2-(dimethylamino)-7-methylquinazolin-4-yl]amino}methyl)phenyl]piperidine-4-carboxamide

N-[4-({[2-(dimethylamino)-7-methylquinazolin-4-yl]amino}methyl)phenyl]piperidine-4-carboxamide (200 mg, 0.48 mmol) was N-benzylated according to procedure D (step 4) using 4-chloro-3-fluorobenzaldehyde and subsequently converted to the bis-HCl salt to give the product (176 mg, yield, 58%). MS (ESI) m/z 561.4.

### Preparation (2-N-methylamino-7-methyl-quinazolin-4-yl)-(4-nitro-benzyl)-amine

To a stirred solution of 4-nitrobenzylamine hydrochloride (444 mg, 2.36 mol) and triethylamine (1 mL) in CHCl₃ (5 mL) at rt was added 7-methyl-2,4-dichloroquinazoline (500 mg, 2.36 mol). The mixture was stirred for one hour. After TLC showed the complete disappearance of the 2,4-dichloroquinazoline CHCl₃ (20 mL) and water (15 mL). The layers were separated and the chloroform layer was dried over MgSO₄. After removal of MgSO₄ by filtration and evaporation of solvents the crude (2-Chloro-7-methyl-quinazolin-4-yl)-(4-nitro-benzyl)-amine (660 mg, yield 85%) was taken forward without further purification.

The (2-Chloro-7-methyl-quinazolin-4-yl)-(4-nitro-benzyl)-amine (600 mg, 1.83 mmol) was taken up in a round bottom flask and was dissolved in DMF (6 mL) and methylamine hydrochloride (2 g, 29.6 mmol, 16 eq) was added. The mixture was heated over one hour to 100° C. After reaction was completed the solvents were removed in vacuo and the mixture was basified with 5 N NaOH and extracted with TEA/THF mixtures. The combined org layers were dried over MgSO₄. Filtration and distillation under reduced pressure led to the crude material, which was purified by column chromatography (CH₂Cl₂/MeoH/NH₃) to give the product (540 mg, yield, 91%). MS (ESI) m/z 324.

### Example 264

### Preparation of 6-chloro-N-[4-({[7-methyl-2-(methylamino)quinazolin-4-yl]amino}methyl)phenyl]nicotinamide

The (2-N-dimethylamino-7-methyl-quinazolin-4-yl)-(4-nitro-benzyl)-amine (540 mg, 1.67 mmol) was dissolved in THF (30 mL) and Pd—C (10% wet) (100 mg) was added. and the mixture hydrogenated under 1 atm pressure for 24 h. When completed MeOH (500 mL) were added and the mixture was filtered over diatomaceous earth. The solvent were removed in vacuum to obtain (4-Amino-benzyl)-(2-methylamino-7-methyl-quinazolin-4-yl)-amine, which was suspended in THF (10 mL) and NEt₃ (0.4 mL) and cooled to 0 C. 6-cloronicontinoyl chloride (280 mg, 1.59 mmol) was added. After completion 1N NaOH (0.5 mL) was added and the layers were separated. The org layer was extracted twice with THF/ethyl acetate (10 mL) and the combined organic layers were dried over MgSO₄. The crude material was purified by column chromatography using CH₂Cl₂/MeOH/NH₃ to give product (230 mg, yield, 31%). MS (ESI) m/z 433.2.

### Preparation (2-N-methylamino-8-methyl-quinazolin-4-yl)-(4-nitro-benzyl)-amine

To a stirred solution of 4-nitrobenzylamine hydrochloride (832 mg, 4.41 mmol) and triethylamine (1 mL) in CHCl₃ (5 mL) at rt was added 8-methyl-2,4-dichloroquinazoline (850 mg, 4.01 mmol). TLC showed the complete disappearance of the 2,4-dichloroquinazoline after two hours, and a white solid precipitated. Water (15 mL) was added and the solid was collected by filtration and washed with water (0.5 mL). After drying over night at 50 C/14 mbar the 2-Chloro-8-methyl-quinazolin-4-yl)-(4-nitro-benzyl)-amine (900 mg, yield 68%) was obtained as white solid. MS (ESI) m/z 329 (is taken forward without further purification).

The (2-Chloro-8-methyl-quinazolin-4-yl)-(4-nitro-benzyl)-amine (700 mg, 2.12 mmol) was taken up in a sealed tube and was suspended in 2M H₂NMe in THF-solution (6 mL) and was heated overnight to 100° C. After reaction was completed the solvents were removed in vacuo and the crude material was purified by column chromatography (CH₂Cl₂ MeOH NH₃) to give the (2-N-dimethylamino-8-methyl-quinazolin-4-yl)-(4-nitro-benzyl)-amine (150 mg, yield, 21%). MS (ESI) m/z 324.

### Preparation of 4-Amino-benzyl)-(2-methylamino-8-methyl-quinazolin-4-yl)-amine

The (2-methylamino-7-methyl-quinazolin-4-yl)-(4-nitro-benzyl)-amine (540 mg, 1.67 mmol) was dissolved in THF (30 mL) and Pd—C (10% wet) (100 mg) was added and the mixture hydrogenated under 1 atm pressure for 24 h. When completed MeOH (500 mL) were added and the mixture was filtered over diatomaceous earth. The solvent were removed in vacuum to obtain (4-Amino-benzyl)-(2-methylamino-8-methyl-quinazolin-4-yl)-amine (72 mg, yield, 50%).

### Example 265

### Preparation of 4-bromo-N-[4-({[8-methyl-2-(methylamino)quinazolin-4-yl]amino}methyl)phenyl]benzamide-TFA salt

In a vial equipped with stirring bar was dissolved (4-Amino-benzyl)-(2-methylamino-8-methyl-quinazolin-4-yl)-amine (13 mg, 0.06 mmol) and NEt₃ (0.03 mL) in THF (1.3 mL) The mixture was stirred at rt and 4-bromobenzoyl chloride (50 mg, 0.28 mmol) was added. After completion of the reaction, the solvents were removed by nitrogen purge and the residue was dissolved in DMSO (2 mL), filtered and injected to a preparative HPLC (high pressure liquid chromatography) using ACN/water/TFA₃-gradients. The product (9 mg, yield, 25%) was obtained after solvent removal. MS (ESI) m/z 476.

### Example 266

### Preparation of 4-cyano-N-[4-({[8-methyl-2-(methylamino)quinazolin-4-yl]amino}methyl)phenyl]benzamide-TFA salt

In a vial equipped with stirring bar was dissolved (4-Amino-benzyl)-(2-methylamino-8-methyl-quinazolin-4-yl)-amine (13 mg, 0.06 mmol) and NEt₃ (0.03 mL) in THF (1.3 mL). The mixture was stirred at rt and 4-cyanobenzoyl chloride (50 mg, 0.28 mmol) was added. After completion of the reaction, the solvents were removed by nitrogen purge and the residue was dissolved in DMSO (2 mL), filtered and injected to a preparative HPLC (high pressure liquid chromatography) using ACN/water/TFA₃-gradients. The product (8 mg, yield, 25%) was obtained after solvent removal. MS (ESI) m/z 423.

### Preparation of 2-chloro-7-methyl-N-[(1S)-1-(4-nitrophenyl)ethyl]quinazolin-4-amine

To the mixture of (S)-∝-methyl-4-nitrobenzylamine (1.14 grams, 4.69 mmol) and tetrahydrofuran (15 mL) was added triethylamine (2.61 mL) and 7-methyl-2,4-dichloroquinazoline at 0° C. then the mixture was stirred at room temperature overnight. The solvent was removed under reduced pressure and the residue was diluted with methylene chloride then washed with water and brine. Dried over sodium sulfate, filtered, concentrated and dried to give the product (1.7 grams, yield, 100%). MS (ESI) m/z 343.1.

### Preparation of N²,N²,7-trimethyl-N⁴-[(S)-1-(4-nitrophenyl)ethyl]quinazoline-2,4-diamine

To the 2-chloro-7-methyl-N-[(1S)-1-(4-nitrophenyl)ethyl]quinazolin-4-amine (1.6 grams, 4.65 mmol) and 2-propanol/THF (1:1) 16 mL was added dimethylamine. HCl and heated under reflux overnight. Added methylene chloride and water, collected organic and the solvent was removed under vacuo then the residue was purified by combi flash chromatography using 10% MeOH in CH₂Cl₂ to give the product (1.2 g, yield, 73%). MS (ESI) m/z 352.2.

### Preparation of N⁴-[(1S)-1-(4-aminophenyl)ethyl]-N²,N²,7-trimethylquinazoline-2,4-diamine

To the mixture of N²,N²,7-trimethyl-N⁴-[(1S)-1-(4-nitrophenyl)ethyl]quinazoline-2,4-diamine (1.1 g, 3.13 mmole) and THF/MeOH (1:1) (14 mL) was added of 10% Pd/C (1.1 g.) is stirred overnight under balloon pressure (H₂). Filtration thru diatomaceous earth and removal of solvent afforded the product. (995 mg, yield, 99%). MS (ESI) m/z 322.2.

### Preparation of tert-butyl 4-({[4-((1S)-1-{[2-(dimethylamino)-7-methylquinazolin-4-yl]amino}ethyl)phenyl]amino}carbonyl)piperidine-1-carboxylate

To the boc-isonipecotic acid (493 mg, 2.15 mmol) acid in DMF (3 mL) was added HOBT (580 mg, 4.29 mmol) and EDAC (618 mg, 3.23 mmol) then the mixture was stirred at room temperature for 1 hr. and added N⁴-[(1S)-1-(4-aminophenyl)ethyl]-N²,N²,7-trimethylquinazoline-2,4-diamine (460 mg, 1.43 mol) and stirred overnight. The solvent was removed and purified by combi flask chromatography using 10% MeOH in CH₂Cl₂ to give the product (430 mg, yield, 56%). MS (ESI) m/z 533.4.

### Example 267

### Preparation of N-[4-((1S)-1-{[2-(dimethylamino)-7-methylquinazolin-4-yl]amino}ethyl)phenyl]piperidine-4-carboxamide

To the tert-butyl 4-({[4-((1S)-1-{[2-(dimethylamino)-7-methylquinazolin-4-yl]amino}ethyl)phenyl]amino}carbonyl)piperidine-1-carboxylate (390 mg, 0.733 mmol) and Chloroform (3 mL) was added trifluoroacetic acid (565 uL) at 0° C. and stirred at room temp. for 2 hrs. The solvent was removed and the residue was basified with 1N NaOH and the product was collected by filtration (280 mg; yield, 88%). MS (ESI) m/z 433.4.

### Example 268

### Preparation of 1-(3,4-difluorobenzyl)-N-[4-((1S)-1-{[2-(dimethylamino)-7-methylquinazolin-4-yl]amino}ethyl)phenyl]piperidine-4-carboxamide

To the [4-((1S)-1-{[2-(dimethylamino)-7-methylquinazolin-4-yl]amino}ethyl)phenyl]piperidine-4-carboxamide (10 mg, 0.231 mmol) in MeOH (1.5 mL) was added 3,4-difluorobenzaldehyde (66 mg, 0.462 mmol) was added NaBH₃CN (29 mg, 0.462 mmol) and ZnCl₂ (32 mg, 0.231 mmol). The mixture was stirred for 4 hrs and the solvent was removed then purified by combi flask chromatography using 10% MeOH in CH₂Cl₂ then dissolved in MeOH (1 mL) and added 4N HCl in Dioxane (1 mL). Stirred for 1 hr. and the solvent was removed and the residue was triturated with ether to give the product as a bis hydrochloride salt. (78 mg, yield, 60%). MS (ESI) m/z 280.2; HRMS: calcd for C32H36F2N6O+H+, 559.29914; found (ESI-FTMS, [M+H]1+), 559.30045.

### Example 269

### Preparation of (S)-6-chloro-N-(4-(1-(2-(dimethylamino)-7-methylquinazolin-4-ylamino)ethyl)phenyl)nicotinamide Step 1—(S)-tert-butyl 1-(4-nitrophenyl)ethylcarbamate

The (S) alpha methyl-4-nitrobenzylamine hydrochloride (7.4 g, 36.6 mmol) was dissolved in THF (100 mL) and a mixture of triethylamine (excess) and di tert butyl dicarbonate (12.0 g, 55.0 mmol) was added. The reaction mixture was heated at 50° C. overnight. At the end, reaction mixture was quenched with water (25 mL) and extracted with ethyl acetate. The organics were dried over magnesium sulfate, filtered and concentrated in vacuo. The oil crystallized upon standing and was collected with hexanes and the white crystals were dried in a vacuum oven to yield (S)-tert-butyl 1-(4-nitrophenyl)ethylcarbamate (9.2 g; 95%; (M−H)-266.2).

### Step 2—(S)-tert-butyl 1-(4-aminophenyl)ethylcarbamate

To a solution of (S)-tert-butyl 1-(4-nitrophenyl)ethylcarbamate (9.2 g, 34.6 mmol) in ethanol (184 mL) and water (92 mL) was treated with iron powder (5.5 g, 100 mol) and ammonium chloride (16.1 g, 303 mmol) and the mixture was refluxed overnight. The reaction was filtered through diatomaceous earth and the filtrate was treated with water and extracted with ethyl acetate. The organics were dried with potassium carbonate filtered and concentrated in vacuo. The resulting oil was dissolved in dichloromethane and passed through a pad of hydrous magnesium silicate and concentrated in vacuo to yield (S)-tert-butyl 1-(4-aminophenyl)ethylcarbamate as a yellow oil (8.1 g; 98%; (M+H)−237.2).

### Step-3 (S)-tert-butyl 1-(4-(6-chloronicotinamido)phenyl)ethylcarbamate

To a solution of (S)-tert-butyl 1-(4-aminophenyl)ethylcarbamate (440 mg, 1.86 mmol) in dichloromethane (7.0 mL) and triethylamine (excess) was added 6-chloronicotinoyl chloride (380 mg, 2.2 mmol) and the reaction stirred at room temperature overnight. White precipitate formed and was collected by filtration, the solids washed with dichloromethane and dried in a vacuum oven to yield (S)-tert-butyl 1-(4-(6-chloronicotinamido)phenyl)ethylcarbamate (600 mg; 86%; (M+H)−376.1).

### Step-4 (S)—N-(4-(1-aminoethyl)phenyl)-6-chloronicotinamide

(S)-tert-butyl 1-(4-(6-chloronicotinamido)phenyl)ethylcarbamate (600 mg, 1.6 mmol) was dissolved in dichloromethane (5.0 mL) and excess TFA (ca. 5.0 mL) was added and stirred at room temperature for 4 h. Concentrated in vacuo to an oil then dissolved in water and filtered off insolubles. The filtrate was basified with 5N sodium hydroxide to precipitate a white solid. Collected by filtration and wash with water and dried in vacuum oven to yield (S)—N-(4-(1-aminoethyl)phenyl)-6-chloronicotinamide (270 mg; 61%; (M−H)−274.1).

### Step-5 (S)-6-chloro-N-(4-(1-(2-chloro-7-methylquinazolin-4-ylamino)ethyl)phenyl)nicotinamide

To a solution of (S)—N-(4-(1-aminoethyl)phenyl)-6-chloronicotinamide (250 mg, 0.9 mmol) in DMF (3.0 mL) and triethylamine was added 2,4-dichloro-7-methylquinazoline (215 mg, 1.0 mmol) and stirred at room temperature overnight. The reaction was poured into ice/water to precipitate a solid. Separated solid was collected by filtration and wash with water. Purified by Dichloromethane/hydrous magnesium silicate filtration and concentrated in vacuo to yield (S)-6-chloro-N-(4-(1-(2-chloro-7-methylquinazolin-4-ylamino)ethyl)phenyl)nicotinamide as a yellow foam (400 mg; 98%; (M+H)−452.2).

### Step-6 (S)-6-chloro-N-(4-(1-(2-(dimethylamino)-7-methylquinazolin-4-ylamino)ethyl)phenyl)nicotinamide

(S)-6-chloro-N-(4-(1-(2-chloro-7-methylquinazolin-4-ylamino)ethyl)phenyl)nicotinamide (400 mg, 0.88 mmol) was dissolved DMF (3.0 mL) and added excess dimethylamine hydrochloride (ca. 400 mg) and microwave at 110° C. for 10 min. Cooled and poured onto ice/water to precipitate a white solid. Collect by filtration washed and dried crude in vacuum oven. Purified by silica gel column to yield (S)-6-chloro-N-(4-(1-(2-(dimethylamino)-7-methylquinazolin-4-ylamino)ethyl)phenyl)nicotinamide dihydrochloride salt (240 mg; 54%; (M+H) 461.2).

### Example 270

### Preparation of (R)-6-chloro-N-(4-(1-(2-(dimethylamino)-7-methylquinazolin-4-ylamino)ethyl)phenyl)nicotinamide

This compound was prepared by following the procedure as outlined in Example 270, steps 1-5 starting from 7-methyl-2,4-dichloroquinazoline.

### Step-6 (R)-6-chloro-N-(4-(1-(2-(dimethylamino)-7-methylquinazolin-4-ylamino)ethyl)phenyl)nicotinamide

Following the procedure as outlined in Example 270, step 6 and starting with (R)-6-chloro-N-(4-(1-(2-chloro-7-methylquinazolin-4-ylamino)ethyl)phenyl)nicotinamide (380 mg, 0.84 mmol) afforded (R)-6-chloro-N-(4-(1-(2-(dimethylamino)-7-methylquinazolin-4-ylamino)ethyl)phenyl)nicotinamide dihydrochloride salt (270 mg; 49%; (M+H) 461.2).

### Example 271

### Preparation of (S)—N-(4-(1-(2-(dimethylamino)-7-methylquinazolin-4-ylamino)ethyl)phenyl)-4-fluorobenzamide

### Step-1 (S)-tert-butyl 1-(4-(4-fluorobenzamido)phenyl)ethylcarbamate

This compound was prepared by following the procedure as outlined in Example 270, step 3. Starting with (S)-tert-butyl 1-(4-aminophenyl)ethylcarbamate (500 mg, 2.12 mmol) and 4-fluorobenzoyl chloride (0.3 mL, 2.5 mmol) the reaction afforded (S)-tert-butyl 1-(4-(4-fluorobenzamido)phenyl)ethylcarbamate (550 mg; 73%; (M−H) 357.3).

### Step-2 (S)—N-(4-(1-aminoethyl)phenyl)-4-fluorobenzamide

This compound was prepared by following the procedure as outlined in Example 270, step 4. Starting with (S)-tert-butyl 1-(4-(4-fluorobenzamido)phenyl)ethylcarbamate (500 mg, 1.4 mmol) afforded (S)—N-(4-(1-aminoethyl)phenyl)-4-fluorobenzamide (320 mg; 89%; (M−H) 257.2).

### Step-3 (S)—N-(4-(1-(2-chloro-7-methylquinazolin-4-ylamino)ethyl)phenyl)-4-fluorobenzamide

This compound was prepared by following the procedure as outlined in Example 270, step 5. Starting with (S)—N-(4-(1-aminoethyl)phenyl)-4-fluorobenzamide (250 mg, 0.96 mmol) afforded (S)—N-(4-(1-(2-chloro-7-methylquinazolin-4-ylamino)ethyl)phenyl)-4-fluorobenzamide (420 mg; 99%; (M+H) 435.2).

### Step-4 (S)—N-(4-(1-(2-(dimethylamino)-7-methylquinazolin-4-ylamino)ethyl)phenyl)-4-fluorobenzamide

This compound was prepared by following the procedure as outlined in Example 270, step 5. Starting from (S)—N-(4-(1-(2-chloro-7-methylquinazolin-4-ylamino)ethyl)phenyl)-4-fluorobenzamide (400 mg, 0.92 mmol) afforded (S)—N-(4-(1-(2-(dimethylamino)-7-methylquinazolin-4-ylamino)ethyl)phenyl)-4-fluorobenzamide hydrochloride salt (250 mg; 56%; (M+H) 444.3).

### Example 272

### Preparation of (R)—N-(4-(1-(2-(dimethylamino)-7-methylquinazolin-4-ylamino)ethyl)phenyl)-4-fluorobenzamide

This compound was prepared by following the procedure as outlined in Example 272, steps 1-3.

### Step-4 (R)—N-(4-(1-(2-(dimethylamino)-7-methylquinazolin-4-ylamino)ethyl)phenyl)-4-fluorobenzamide

Following the same procedure as outlined in Example 272 step 4 and starting from (R)—N-(4-(1-(2-chloro-7-methylquinazolin-4-ylamino)ethyl)phenyl)-4-fluorobenzamide (380 mg, 0.87 mmol) afforded (R)—N-(4-(1-(2-(dimethylamino)-7-methylquinazolin-4-ylamino)ethyl)phenyl)-4-fluorobenzamide hydrochloride salt (270 mg; 61%; (M+H) 444.3).

### Example 273

### Preparation of (S)-6-chloro-N-(4-(1-(2-(dimethylamino)-8-methylquinazolin-4-ylamino)ethyl)phenyl)nicotinamide

This compound was prepared by following the procedure as outlined in Example 270, steps 1-4.

### Step-1 (S)-6-chloro-N-(4-(1-(2-chloro-8-methylquinazolin-4-ylamino)ethyl)phenyl)nicotinamide

Following the same procedure as outlined in Example 270 step 5 and starting from (S)—N-(4-(1-aminoethyl)phenyl)-6-chloronicotinamide (100 mg, 0.36 mmol) and 2,4-dichloro-8-methylquinazoline (77 mg, 0.36 mmol) afforded (S)-6-chloro-N-(4-(1-(2-chloro-8-methylquinazolin-4-ylamino)ethyl)phenyl)nicotinamide (150 mg; 71%; (M+H) 452.2).

### Step-2 (S)-6-chloro-N-(4-(1-(2-(dimethylamino)-8-methylquinazolin-4-ylamino)ethyl)phenyl)nicotinamide

Following the same procedure as outlined in Example 270 step 6. (S)-6-chloro-N-(4-(1-(2-chloro-8-methylquinazolin-4-ylamino)ethyl)phenyl)nicotinamide (100 mg 0.22 mmol) afforded (S)-6-chloro-N-(4-(1-(2-(dimethylamino)-8-methylquinazolin-4-ylamino)ethyl)phenyl)nicotinamide dihydrochloride salt (75 mg; 64%; (M+H) 461.3).

### Example 274

### Preparation of (S)-6-chloro-N-(4-(1-(2-(dimethylamino)-6-methylquinazolin-4-ylamino)ethyl)phenyl)nicotinamide

This compound was prepared by following the procedure as outlined in Example 270, steps 1-4.

### Step-1 (S)-6-chloro-N-(4-(1-(2-chloro-6-methylquinazolin-4-ylamino)ethyl)phenyl)nicotinamide

Following the same procedure as outlined in Example 270 step 5 and starting from (S)—N-(4-(1-aminoethyl)phenyl)-6-chloronicotinamide (100 mg, 0.36 mmol) and 2,4-dichloro-6-methylquinazoline (77 mg, 0.36 mmol) afforded (S)-6-chloro-N-(4-(1-(2-chloro-6-methylquinazolin-4-ylamino)ethyl)phenyl)nicotinamide. (110 mg; 70%; (M+H) 452.2).

### Step-6 (S)-6-chloro-N-(4-(1-(2-(dimethylamino)-6-methylquinazolin-4-ylamino)ethyl)phenyl)nicotinamide

Following the same procedure as outlined in Example 270 step 6 and starting from (S)-6-chloro-N-(4-(1-(2-chloro-8-methylquinazolin-4-ylamino)ethyl)phenyl)nicotinamide (100 mg 0.22 mmol) afforded (S)-6-chloro-N-(4-(1-(2-(dimethylamino)-6-methylquinazolin-4-ylamino)ethyl)phenyl)nicotinamide.dihydrochloride salt (90 mg; 77%; (M+H) 461.3).

### Example 275

### Preparation of (S)-4-fluoro-N-(4-(1-(8-methyl-2-(methylamino)quinazolin-4-ylamino)ethyl)phenyl)benzamide

This compound was prepared by following the procedure as outlined in Example 272, steps 1-2.

### Step 1 (S)—N-(4-(1-(2-chloro-8-methylquinazolin-4-ylamino)ethyl)phenyl)-4-fluorobenzamide

This compound was prepared by following the procedure as outlined in Example 270, step 5. Starting with (S)—N-(4-(1-aminoethyl)phenyl)-4-fluorobenzamide (280 mg, 1.07 mmol) and 2,4-dichloro-8-methylquinazoline (230 mg, 1.07 mmol afforded (S)—N-(4-(1-(2-chloro-8-methylquinazolin-4-ylamino)ethyl)phenyl)-4-fluorobenzamide (470 mg; 99%; (M+H) 435.1).

### Step 2 (S)—N-(4-(1-(2,8-dimethylquinazolin-4-ylamino)ethyl)phenyl)-4-fluorobenzamide

This compound was prepared by following the procedure as outlined in Example 270, step 5. Starting from (S)—N-(4-(1-(2-chloro-8-methylquinazolin-4-ylamino)ethyl)phenyl)-4-fluorobenzamide (200 mg, 0.46 mmol) and methylamine hydrochloride (excess) afforded (S)—N-(4-(1-(2,8-dimethylquinazolin-4-ylamino)ethyl)phenyl)-4-fluorobenzamide dihydrochloride salt (115 mg; 50%; (M+H) 430.2).

### Example 276

### Preparation of (R)-4-fluoro-N-(4-(1-(8-methyl-2-(methylamino)quinazolin-4-ylamino)ethyl)phenyl)benzamide

This compound was prepared from 8-methyl-2,4-dichloroquinazoline by following the procedure as outlined in Example 270, steps 1 to 5.

### Preparation of (R)-4-fluoro-N-(4-(1-(8-methyl-2-(methylamino)quinazolin-4-ylamino)ethyl)phenyl)benzamide

Starting from (R)—N-(4-(1-(2-chloro-8-methylquinazolin-4-ylamino)ethyl)phenyl)-4-fluorobenzamide (310 mg, 0.71 mmol) and methylamine hydrochloride (excess) afforded (S)—N-(4-(1-(2,8-dimethylquinazolin-4-ylamino)ethyl)phenyl)-4-fluorobenzamide hydrochloride salt (101 mg; 27%; (M+H) 430.2).

### Biological Assays:

Compounds of the present invention can be tested according to the protocol described.

Background cell line: SW480 colorectal carcinoma DNA constructs:

1) Tcf-4-Luc: Tcf-4 binding sites driving Firefly Luciferase in the pGL3 vector—to measure the activity of β-catenin/Tcf-4;

2) SV40-Luc: SV40 binding site driving Firefly Luciferase in the pGL3 vector—as a control for non-specific inhibitors; and

3) SV40-R-Luc: SV40 binding site driving Renilla Luciferase in the pGL3 vector—as an internal control for cell number and toxicity.

This screen uses 3 cell lines derived from SW480 and selected to contain the above luciferase reporters integrated into their chromosomes as follows:

Tcf 33.13—Tcf-4-Luc and SV40-R-Luc

Tcf 22C11—Tcf-4-Luc and SV40-R-Luc

SV 5A8—SV40-Luc and SV40-R-Luc

Cells: Tcf22C11 and SV5A8 were grown in the presence of 500 μg/ml G418 and Tcf 33.13 was grown in the presence of 500 μg/ml G418+125 μg/ml Zeocin to maintain the integrated reporters. The cells were trypsinized and plated at 1×10⁴ cells/well in opaque 96 well plates.

Compounds: 20-24 hours after cell plating, compounds were added at titrated concentrations between 10 μg/ml and 10 μg/ml. The compounds were diluted as follows: The compounds were stored frozen in dimethyl sulfoxide (DMSO) at 10 μg/ml. Compounds were initially titrated in DMSO to make a 1000×DMSO stock solution. Compounds were then diluted 1:25 into media/DMSO (DMEM+10% FBS+9% DMSO). Using the Biomek Multimek, the diluted compounds were mixed well and immediately added to the plated cells in a 1:40 dilution. The final concentration of DMSO in the assay plates was 0.316% DMSO.

Assay Readout: The luciferase signal was detected using a luciferase detection kit from Promega. Until December 2003, Promega's Dual-Luciferase® Reporter (DLR™) Assay System was used to detect luciferase activity. Briefly, after 24 hours of incubation with compound, culture media was removed by aspiration and 20 ul of passive lysis buffer (Promega) per well was added. The plates were shaken for 15 min. Firefly substrate was then added and the resulting luminescence was immediately read on a CCD camera imaging device or a luminometer. After quantifying the firefly luminescence, the reaction was quenched, and the Renilla luciferase reaction was initiated simultaneously by adding Stop & Glo® Reagent to the same sample. Renilla luciferase was then quantified using the CCD camera or luminometer.

Promega's Dual-Glo™ Luciferase Assay System can be used for luciferase detection. It was similar to the DLR Assay, however, unlike the DLR which has to be read immediately due to decaying signal, the signal in the Dual-Glo™ Luciferase Assay was stable for up to 2 hours. Briefly, after 24 hours of incubation with compound, culture media was removed by aspiration and 75 ul of fresh medium was added. Add 75 ul of Dual-Glo Luciferase Reagent (Promega) to each well and mix by shaking for 10 minutes on a plate shaker. Then, measure the firefly luminescence on a CCD camera imaging device or a luminometer. After quantifying the firefly luminescence, add 75 ul of Dual-Glo Stop & Glo Reagent (Promega) to each well and mix for 10 minutes. Renilla luciferase was then quantified using the CCD camera or luminometer.

This assay was meant to confirm primary leads and determine IC50s. Compounds were tested at titrated dilutions. The raw data for this assay was reported in protocol 2463-B. Protocol 2463-C contains the IC50 calculations based on the raw data. The data calculations were done as follows:

The data was expressed as % of control for both Firefly and Renilla Luciferase. The ratio of FF/Renilla (F/R) was then calculated and averaged between duplicate plates. The data was then plotted using LSW Data Analysis in Microsoft Excel. IC50s were determined by plotting the data in the LSW Data Analysis program using model 33. The IC50s (nM) for all three cell lines were reported.

### REFERENCES

1. Morin, P. J., Vogelstein, B., and Kinzler, K. W. (1996) Apoptosis and APC in colorectal tumorigenesis. Proc. Natl. Acac. Sci. 93, 7950-7954.
2. Munemitsu, S., Albert, I., Souza, B., Rubinfield, B. and Polakis, P. (1995) Regulation of intracellular β-catenin levels by the adenomatous polyposis coli (APC) tumor-suppessor protein. Proc. Natl. Acad. Sci. 92, 3046-3050.
3. Korinek, V., Barker, N., Morin, P. J., Wichen, D., Weger, R., Kinzler, K. W., Vogelstien, B., Clevers, H. (1997) Constitutive transcriptional activation by a β-catenin-Tcf complex in APC 1/1 colon carcinoma. Science 275, 1784-1787.
4. Morin, P. J., Spark, A. B., Korinek, V., Barker, N., Clevers, H., Vogelstein, B., Kinzler, K. W. (1997) Activation of b-catenin-Tcf signaling in colon cancer by mutations in β-catenin or APC. Science 275, 1787-1790.
5. Bienz, M., Clevers, H. (2000) Linking colorectal cancer to Wnt signaling. Cell 103, 311-320.

RKE/b-catenin in vivo growth assay: Athymic nude balb/c nu/nu mice (Charles River Laboratories), housed according to the Institute of Laboratory Animal Resources standards, age 8-10 weeks and weighing 20-25 grams were injected subcutaneously on the right side with 5 million RKE/b-catenin cells derived from tissue culture in a 0.2 cc.volume. 4 days following inoculation, the mice were randomized into groups of 10-12 mice, with the mean tumor volume approximately 200 mg. Treatment begins on day 0 of staging and the tumor size was monitored twice weekly with the use of calipers and the formula [L×W×W/2]. Mice were evaluated daily for signs of toxicity.

The compounds of the present invention were tested according to the protocol described. Results were required as IC₅₀ values as shown in Table 1.

TABLE 1

| Example | IC₅₀(22C11)nM | IC₅₀(33.13)nM | IC₅₀(5A8)nM |
|---|---|---|---|
| Example 1 | >6017 | >6017 | NA |
| Example 2 | >2924 | >2924 | NA |
| Example 3 | 690 | 596 | >2984 |
| Example 4 | 680 | 787 | >2984 |
| Example 7 | >2984 | >2984 | >23873 |
| Example 8 | 1771 | 1532 | >5304 |
| Example 9 | 888 | 1069 | 1525 |
| Example 10 | 1188 | 771 | 2194 |
| Example 11 | 2274 | 2771 | >5982 |
| Example 12 | >25158 | >25158 | >25158 |
| Example 13 | 423 | 413 | >778 |
| Example 14 | 891 | 1292 | >2868 |
| Example 15 | >2720 | 941 | >5440 |
| Example 16 | >2763 | 1820 | >2763 |
| Example 17 | 7514 | >6017 | >12035 |
| Example 18 | 1030 | 630 | >5788 |
| Example 19 | >21442 | 12844 | >21442 |
| Example 20 | 1120 | >692 | >1384 |
| Example 21 | 5641 | 3647 | >12241 |
| Example 22 | 1381 | 1232 | >2674 |
| Example 23 | 1875 | 1715 | >5594 |
| Example 24 | 148 | 113 | >602 |
| Example 25 | 2707 | 2965 | >5917 |
| Example 26 | 739 | 614 | >2778 |
| Example 27 | 526 | 402 | >5371 |
| Example 28 | >1389 | 904 | >1389 |
| Example 29 | 988 | 756 | >2803 |
| Example 30 | 2105 | 1866 | >5821 |
| Example 31 | 5951 | 4125 | >22276 |
| Example 32 | 100 | 81 | >577 |
| Example 33 | 1280 | 1715 | >2791 |
| Example 34 | 4194 | 3226 | >11535 |
| Example 35 | >21554 | >21554 | >21554 |
| Example 36 | 1169 | 866 | >5587 |
| Example 37 | >10721 | 5285 | >21442 |
| Example 38 | 1620 | 2052 | >11642 |
| Example 39 | 428 | 217 | >5821 |
| Example 40 | 1620 | 2052 | >11642 |
| Example 41 | 2318 | 1653 | >5768 |
| Example 42 | 9248 | 2936 | >10648 |
| Example 43 | 738 | 492 | >5464 |
| Example 44 | >21952 | >21952 | >21952 |
| Example 45 | 1804 | 1484 | >10626 |
| Example 46 | >20767 | >20767 | >20767 |
| Example 47 | 11965 | 12815 | >24069 |
| Example 48 | >20637 | >20637 | >20637 |
| Example 49 | >2507 | >2507 | >10028 |
| Example 50 | >1254 | 1494 | >5014 |
| Example 51 | 1066 | 987 | >5159 |
| Example 52 | 1649 | 1307 | >22547 |
| Example 53 | 462 | 306 | >2803 |
| Example 54 | 1230 | 846 | >18429 |
| Example 55 | 8653 | 3261 | >18260 |
| Example 56 | 1736 | 2011 | >5170 |
| Example 57 | 923 | 1597 | >10028 |
| Example 58 | 2500 | 2079 | 4193 |
| Example 59 | 229 | 453 | >2334 |
| Example 60 | 5388 | 1954 | >11535 |
| Example 61 | 532 | 327 | >5061 |
| Example 62 | 1111 | 808 | >9776 |
| Example 63 | 2152 | 1191 | >8723 |
| Example 64 | 11069 | 8959 | >10754 |
| Example 65 | 548 | 423 | >2887 |
| Example 66 | 1006 | 649 | >9788 |
| Example 67 | 1207 | 1446 | >10040 |
| Example 68 | 530 | 400 | >4618 |
| Example 69 | 2007 | 1936 | >20137 |
| Example 70 | 2142 | 1476 | >10560 |
| Example 71 | 2986 | 2343 | >11223 |
| Example 72 | 534 | 456 | >4474 |
| Example 73 | 223 | 194 | >681 |
| Example 74 | 950 | 1385 | >8058 |
| Example 75 | 437 | 287 | >4487 |
| Example 76 | 1557 | 2151 | >9172 |
| Example 77 | 2265 | 847 | >8229 |
| Example 78 | 1389 | 955 | >17308 |
| Example 79 | 2351 | 1676 | >15954 |
| Example 80 | 761 | 597 | >9386 |
| Example 81 | >8917 | >8917 | >17835 |
| Example 82 | 872 | 891 | >7964 |
| Example 83 | 511 | 425 | >4046 |
| Example 84 | 434 | 423 | >9306 |
| Example 85 | 771 | 530 | >2217 |
| Example 86 | 6815 | 4192 | >18472 |
| Example 87 | 382 | 256 | >20922 |
| Example 88 | 431 | 335 | >2604 |
| Example 89 | 2138 | 1479 | >20967 |
| Example 90 | 2865 | 1304 | >4832 |
| Example 91 | 1046 | 1222 | >9166 |
| Example 92 | 895 | 884 | >17937 |
| Example 93 | 811 | 855 | >10374 |
| Example 94 | 2592 | 2331 | >20244 |
| Example 95 | 2013 | 1900 | >20162 |
| Example 96 | 497 | 511 | >2541 |
| Example 97 | 499 | 599 | >2557 |
| Example 98 | >2406 | 2958 | >19246 |
| Example 99 | 6825 | 2675 | >17894 |
| Example 100 | 548 | 541 | >17747 |
| Example 101 | 2254 | 2952 | >17604 |
| Example 102 | 1335 | 825 | >9362 |
| Example 103 | 615 | 429 | >4681 |
| Example 104 | 1012 | 666 | >2541 |
| Example 105 | 3781 | 1472 | >8206 |
| Example 106 | 551 | 393 | >9815 |
| Example 107 | 414 | 336 | >4776 |
| Example 108 | 508 | 236 | >5253 |
| Example 110 | 1762 | 1189 | >16799 |
| Example 111 | 238 | 181 | >2523 |
| Example 112 | 249 | 118 | >4907 |
| Example 113 | 1490 | 766 | >3009 |
| Example 114 | 870 | 587 | >5848 |
| Example 115 | 1382 | 991 | >2831 |
| Example 116 | 3445 | 3037 | >21252 |
| Example 117 | 7166 | 4479 | >21858 |
| Example 118 | 942 | 588 | >3009 |
| Example 119 | 1741 | 1273 | >2831 |
| Example 120 | 850 | 795 | >1372 |
| Example 121 | 1724 | 1135 | >2924 |
| Example 122 | 2293 | 1264 | >11273 |
| Example 123 | 5846 | 2930 | >10976 |
| Example 124 | 674 | 332 | >1337 |
| Example 125 | 1582 | 364 | >1331 |
| Example 126 | 1719 | 1073 | >2492 |
| Example 127 | >22449 | >22449 | >22449 |
| Example 128 | 1132 | 708 | >23099 |
| Example 129 | 738 | 754 | >9172 |
| Example 130 | 798 | 699 | >2354 |
| Example 131 | 1041 | 966 | >7866 |
| Example 132 | 1388 | 1138 | >2149 |
| Example 133 | 669 | 793 | >9351 |
| Example 134 | 805 | 782 | >9603 |
| Example 135 | 965 | 853 | >8093 |
| Example 136 | 494 | 365 | >15929 |
| Example 137 | 1261 | 1128 | >17308 |
| Example 138 | 2277 | 2124 | >8421 |
| Example 139 | 1456 | 1416 | >18564 |
| Example 140 | 1416 | 1316 | >8624 |
| Example 141 | 1582 | 1328 | >17738 |
| Example 142 | 573 | 677 | >4275 |
| Example 143 | 1927 | 1466 | >8869 |
| Example 144 | 2269 | 1445 | >8838 |
| Example 145 | 1353 | 1231 | >16673 |
| Example 146 | 1996 | 1767 | >10108 |
| Example 147 | 1573 | 1246 | >5202 |
| Example 148 | 1067 | 1032 | >9172 |
| Example 149 | 606 | 726 | >2527 |
| Example 150 | 268 | 169 | >2401 |
| Example 151 | 606 | 726 | >2527 |
| Example 152 | 1049 | >962 | >3848 |
| Example 153 | 462 | 305 | >4114 |
| Example 154 | 3594 | 4191 | >8611 |
| Example 155 | >2109 | 3486 | >8437 |
| Example 156 | >4121 | 3691 | >8242 |
| Example 157 | >7145 | >3573 | >7145 |
| Example 158 | >4107 | 3850 | >8214 |
| Example 159 | >8242 | >8242 | >8242 |
| Example 160 | 1452 | >2206 | >4412 |
| Example 161 | 1709 | 1518 | >10002 |
| Example 162 | 3579 | 2232 | >9767 |
| Example 163 | 6990 | 6554 | >9506 |
| Example 164 | 5190 | 4313 | >8077 |
| Example 165 | 9637 | 5433 | >9470 |
| Example 166 | >9506 | >9506 | >9506 |
| Example 167 | 919 | 751 | >5145 |
| Example 168 | 2813 | 1284 | >9059 |
| Example 169 | 229 | 453 | >2334 |
| Example 170 | 692 | 806 | >2338 |
| Example 171 | 2941 | 4277 | >19320 |
| Example 172 | 397 | 308 | >2831 |
| Example 173 | >4512 | 4484 | >18048 |
| Example 174 | 436 | 359 | >5081 |
| Example 175 | 712 | 537 | >4681 |
| Example 176 | 556 | 425 | >4681 |
| Example 177 | >5423 | >5423 | >10847 |
| Example 178 | 266 | 245 | >2712 |
| Example 179 | 538 | 924 | >4946 |
| Example 180 | 1459 | 2025 | >5612 |
| Example 181 | 719 | 549 | >2806 |
| Example 182 | 4279 | 4317 | >11615 |
| Example 183 | 548 | 423 | >2887 |
| Example 184 | >21557 | >21557 | >21557 |
| Example 188 | >23286 | >23286 | >23286 |
| Example 189 | 1828 | 1145 | >4566 |
| Example 190 | 948 | 782 | >3009 |
| Example 191 | 405 | 411 | >1447 |
| Example 192 | 1716 | 1080 | >3009 |
| Example 193 | 829 | 492 | >2894 |
| Example 194 | 848 | 541 | >5775 |
| Example 195 | 389 | 270 | >1158 |
| Example 196 | 223 | 227 | >1401 |
| Example 197 | 376 | 382 | >2390 |
| Example 198 | 346 | 253 | >2500 |
| Example 199 | 593 | 454 | >2910 |
| Example 200 | 804 | 491 | >4565 |
| Example 201 | 1308 | 664 | >4994 |
| Example 202 | 505 | 300 | >2831 |
| Example 203 | 590 | 579 | >9889 |
| Example 204 | 767 | 677 | >10318 |
| Example 205 | >2502 | 3154 | >10008 |
| Example 206 | 524 | 291 | >2662 |
| Example 207: | 1779 | 635 | >10604 |
| Example 208: | 621 | 322 | >2656 |
| Example 209: | 659 | 378 | >2744 |
| Example 210: | 1377 | 825 | >5637 |
| Example 211 | 558 | 396 | >2732 |
| Example 212 | 3098 | 2677 | >12908 |
| Example 213 | 1714 | 1388 | >12350 |
| Example 214 | 452 | 379 | >1090 |
| Example 215 | 1961 | 3194 | >12009 |
| Example 216 | 464 | 315 | >3219 |
| Example 217 | 1858 | 3682 | >16324 |
| Example 218 | 1280 | 939 | >15229 |
| Example 219 | 1800 | 1425 | >14626 |
| Example 220 | 1136 | 682 | >8510 |
| Example 221 | 1235 | 1060 | >8202 |
| Example 222 | 918 | 650 | >7879 |
| Example 223 | 4465 | 2665 | >15609 |
| Example 224 | 1000 | 781 | >9735 |
| Example 225 | 273 | 237 | >2580 |
| Example 226 | 866 | 754 | >9282 |
| Example 227 | 1146 | 736 | >10028 |
| Example 228 | 1903 | 2208 | >4641 |
| Example 229 | 2190 | 2322 | >4615 |
| Example 230 | 329 | 299 | >752 |
| Example 231 | 645 | 506 | >2818 |
| Example 232 | 408 | 292 | >2468 |
| Example 233 | >4350 | >2175 | >4350 |
| Example 234 | 478 | 428 | >6783 |
| Example 235 | 451 | 448 | >5170 |
| Example 236 | 511 | 525 | >5014 |
| Example 237 | 888 | 829 | >5014 |
| Example 238 | 284 | 255 | >1312 |
| Example 239 | 204 | 193 | >1274 |
| Example 240 | 900 | 793 | >3030 |
| Example 241 | 1032 | 813 | >23446 |
| Example 242 | 318 | 336 | >5594 |
| Example 243 | 1923 | 1675 | >23118 |
| Example 244 | 650 | 962 | >10534 |
| Example 245 | 754 | 809 | >9986 |
| Example 246 | 1831 | 1934 | >20055 |
| Example 247 | 539 | 467 | >4197 |
| Example 248 | >20055 | >20055 | >20055 |
| Example 249 | 1479 | 1173 | >10534 |
| Example 250 | 373 | 213 | >9282 |
| Example 251 | 262 | 215 | >8339 |
| Example 252 | 568 | 332 | >8339 |
| Example 253 | 390 | 255 | >4058 |
| Example 254 | 168 | 85 | >4590 |
| Example 255 | 196 | 106 | >2295 |
| Example 256 | 967 | 706 | >8671 |
| Example 257 | 1188 | 906 | >8077 |
| Example 258 | 521 | 215 | >7886 |
| Example 259 | 573 | 415 | >8103 |
| Example 260 | 215 | 203 | >7867 |
| Example 261 | 347 | 328 | >4170 |
| Example 262 | 213 | 184 | >4590 |
| Example 263 | 422 | 410 | >1972 |
| Example 264 | 650 | 413 | >1444 |
| Example 265 | 1141 | 933 | >4234 |
| Example 266 | 1100 | 962 | >4660 |
| Example 268 | 215 | 203 | >7867 |
| Example 269 | 439 | 319 | >11273 |
| Example 270 | 429 | 372 | >10052 |
| Example 271 | 970 | 1079 | >5637 |
| Example 272 | 894 | 986 | >5423 |
| Example 273 | >2341 | 3951 | >9365 |
| Example 274 | 1011 | 1837 | >9365 |
| Example 275 | 522 | 549 | >1244 |
| Example 276 | 653 | 494 | >2488 |

All references cited herein are incorporated herein by reference in their entirety and for all purposes to the same extent as if each individual publication or patent or patent application is specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, analytical results and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

We claim:

1. A compound of formula I, 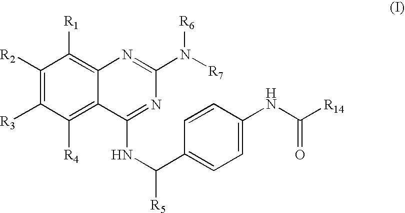
or an enantiomer, diastereomer, tautomer, or pharmaceutically acceptable salt or solvate thereof, wherein the symbols have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
wherein: R₂ and R₃ together with the two contiguous carbon atoms to which R₂ and R₃ are bonded may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
each occurance of Rₐ is independently hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
each occurance of R_{b}, R_{c} and R_{d} is independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
each occurance of Rₑ is alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

2. The compound of claim 1, wherein R₁, R₂, R₃, and R₄ are each independently hydrogen, halogen, cyano, nitro, CF₃, OCF₃, C₁-C₄ alkyl or substituted C₁-C₄ alkyl, C₂-C₆ alkenyl or substituted C₂-C₆ alkenyl, C₂-C₆ alkynyl or substituted C₂-C₆ alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, S(═O)₂ORₑ, NR_{b}R_{c}, NR_{b}S(═O)₂Rₑ, S(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, or NR_{b}C(═O)Rₐ.

3. The compound of claim 2, wherein R₂ and R₃ are each independently hydrogen, halogen, cyano, nitro, CF₃, OCF₃, C₁-C₄ alkyl or substituted C₁-C₄ alkyl, C₂-C₆ alkenyl or substituted C₂-C₆ alkenyl, C₂-C₆ alkynyl or substituted C₂-C₆ alkynyl, C₃-C₇ cycloalkyl or substituted C₃-C₇ cycloalkyl, heterocyclyl or substituted heterocyclyl, aryl or substituted aryl, ORₐ, C(═O)ORₑ, or C(═O)Rₐ.

4. The compound of claim 3, wherein R₆ and R₇ are each independently hydrogen, C₁-C₄ alkyl or substituted C₁-C₄ alkyl, C₃-C₇ cycloalkyl or substituted C₃-C₇ cycloalkyl, or said R₆ and R₇ together with the N to which they are bonded optionally form a heterocycle or substituted heterocycle, in which said heterocycle is fully saturated or partially unsaturated.

5. The compound of claim 4, wherein R₅ is hydrogen or methyl.

6. The compound of claim 5, wherein R₁₄ is heteroaryl or substituted heteroaryl.

7. The compound of claim 5, wherein R₁₄ is aryl or substituted aryl.

8. The compound of claim 5, wherein R₁₄ is heterocycle or substituted heterocycle, in which said heterocycle is fully saturated.

9. The compound of claim 5, wherein R₁₄ is phenyl or substituted phenyl.

10. The compound of claim 5, wherein R₁₄ is pyridinyl or substituted pyridinyl.

11. The compound of claim 5, wherein R₁₄ is piperidinyl or substituted piperidinyl.

12. The compound of claim 1, wherein R₁₄ is 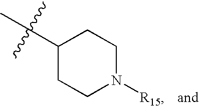
wherein R₁₅ is hydrogen, C₁-C₄ alkyl, C₃-C₇ cycloalkyl, —CH₂-phenyl or —CH₂-substituted phenyl, or —CH₂-heteroaryl or —CH₂-substituted heteroaryl.

13. The compound of claim 5, wherein R₁₄ is —NH-aryl or —NH-substituted aryl.

14. The compound of claim 5, wherein R₁₄ is —NH-phenyl or —NH-substituted phenyl.

15. The compound of claim 1, wherein the compound of formula I is selected from the group consisting of:
5-fluoro-2-methyl-N-[4-({[2-(methylamino)quinazolin-4-yl]amino}methyl)phenyl]benzamide;
2-(benzyloxy)-N-[4-({[2-(methylamino)quinazolin-4-yl]amino}methyl)phenyl]acetamide;
6-chloro-N-[4-({[2-(methylamino)quinazolin-4-yl]amino}methyl)phenyl]nicotinamide;
2-chloro-N-[4-({[2-(methylamino)quinazolin-4-yl]amino}methyl)phenyl]isonicotinamide;
N-[4-({[2-(methylamino)quinazolin-4-yl]amino}methyl)phenyl]quinoline-2-carboxamide;
2-chloro-5-fluoro-N-[4-({[2-(methylamino)quinazolin-4-yl]amino}methyl)phenyl]benzamide;
2-chloro-N-[4-({[2-(methylamino)quinazolin-4-yl]amino}methyl)phenyl]nicotinamide;
2,6-dichloro-5-fluoro-N-[4-({[2-(methylamino)quinazolin-4-yl]amino}methyl)phenyl]nicotinamide;
6-chloro-N-[4-({[6,7-dimethoxy-2-(methylamino)quinazolin-4-yl]amino}methyl)phenyl]nicotinamide;
4-chloro-N-[4-({[2-(methylamino)quinazolin-4-yl]amino}methyl)phenyl]benzamide;
3-chloro-N-[4-({[2-(methylamino)quinazolin-4-yl]amino}methyl)phenyl]benzamide;
4-methyl-N-[4-({[2-(methylamino)quinazolin-4-yl]amino}methyl)phenyl]benzamide;
4-fluoro-N-[4-({[2-(methylamino)quinazolin-4-yl]amino}methyl)phenyl]benzamide;
4-chloro-2-fluoro-N-[4-({[2-(methylamino)quinazolin-4-yl]amino}methyl)phenyl]benzamide;
N-[4-({[2-(methylamino)quinazolin-4-yl]amino}methyl)phenyl]biphenyl-4-carboxamide;
2,4-dichloro-N-[4-({[2-(methylamino)quinazolin-4-yl]amino}methyl)phenyl]benzamide;
4-fluoro-3-methyl-N-[4-({[2-(methylamino)quinazolin-4-yl]amino}methyl)phenyl]benzamide;
4-chloro-N-[4-({[6-methyl-2-(methylamino)quinazolin-4-yl]amino}methyl)phenyl]benzamide;
2,4-dichloro-N-[4-({[6-methyl-2-(methylamino)quinazolin-4-yl]amino}methyl)phenyl]benzamide;
N-[4-({[2-(methylamino)quinazolin-4-yl]amino}methyl)phenyl]-4-(trifluoromethyl)benzamide;
4-cyano-N-[4-({[2-(methylamino)quinazolin-4-yl]amino}methyl)phenyl]benzamide;
N-[4-({[2-(methylamino)quinazolin-4-yl]amino}methyl)phenyl]-4-(trifluoromethoxy)benzamide;
6-chloro-N-[4-({[6,8-dimethyl-2-(methylamino)quinazolin-4-yl]amino}methyl)phenyl]nicotinamide;
4-fluoro-N-[4-({[6-methyl-2-(methylamino)quinazolin-4-yl]amino}methyl)phenyl]benzamide;
4-cyano-N-[4-({[6-methyl-2-(methylamino)quinazolin-4-yl]amino}methyl)phenyl]benzamide;
4-chloro-2-fluoro-N-[4-({[6-methyl-2-(methylamino)quinazolin-4-yl]amino}methyl)phenyl]benzamide;
N-[4-({[6-methyl-2-(methylamino)quinazolin-4-yl]amino}methyl)phenyl]-4-(trifluoromethyl)benzamide;
2-chloro-4-fluoro-N-[4-({[6-methyl-2-(methylamino)quinazolin-4-yl]amino}methyl)phenyl]benzamide;
4-chloro-N-[4-({[6,8-dimethyl-2-(methylamino)quinazolin-4-yl]amino}methyl)phenyl]benzamide;
N-[4-({[6,8-dimethyl-2-(methylamino)quinazolin-4-yl]amino}methyl)phenyl]-4-fluorobenzamide;
6-chloro-N-[4-({[6-methoxy-2-(methylamino)quinazolin-4-yl]amino}methyl)phenyl]nicotinamide;
3,4-difluoro-N-[4-({[6-methyl-2-(methylamino)quinazolin-4-yl]amino}methyl)phenyl]benzamide;
4-chloro-N-[4-({[6-methoxy-2-(methylamino)quinazolin-4-yl]amino}methyl)phenyl]benzamide
2,4-difluoro-N-[4-({[6-methyl-2-(methylamino)quinazolin-4-yl]amino}methyl)phenyl]benzamide;
4-chloro-N-[4-({[6,8-dimethyl-2-(methylamino)quinazolin-4-yl]amino}methyl)phenyl]-2-fluorobenzamide;
N-[4-({[2-(dimethylamino)-6-methylquinazolin-4-yl]amino}methyl)phenyl]-3,4-difluorobenzamide;
3,4-dichloro-N-[4-({[6-methyl-2-(methylamino)quinazolin-4-yl]amino}methyl)phenyl]benzamide;
N-[4-({[2-(dimethylamino)-6-methylquinazolin-4-yl]amino}methyl)phenyl]-4-fluorobenzamide;
3,5-difluoro-N-[4-({[6-methyl-2-(methylamino)quinazolin-4-yl]amino}methyl)phenyl]benzamide;
4-fluoro-N-(4-{[(6-methyl-2-piperidin-1-ylquinazolin-4-yl)amino]methyl}phenyl)benzamide;
N-[4-((2-(diethylamino)-6-methylquinazolin-4-ylamino)methyl)phenyl)-4-fluorobenzamide;
N-(4-((2-(1-azacyclopentyl)-6-methylquinazolin-4-ylamino)methyl)phenyl)-4-fluorobenzamide;
4-fluoro-N-(4((6-methyl-2-piperazin-1-yl)quinazolin-4-ylamino)methyl)phenyl)benzamide;
4-fluoro-N-((6-methyl-2-(4-methylpiperazin-1-yl)quinazolin-4-ylamino)methyl-phenyl)benzamide;
2-fluoro-N-[4-({[6-methyl-2-(methylamino)quinazolin-4-yl]amino}methyl)phenyl]benzamide;
4-fluoro-N-[4-({[6-methyl-2-(4-methylpiperazin-1-yl)quinazolin-4-yl]amino}methyl)phenyl]benzamide;
N-[4-({[6,8-dimethyl-2-(4-methylpiperazin-1-yl)quinazolin-4-yl]amino}methyl)phenyl]-4-fluorobenzamide;
N-{4-[({6,8-dimethyl-2-[(3S)-3-methylpiperazin-1-yl]quinazolin-4-yl}amino)methyl]phenyl}-4-fluorobenzamide;
4-fluoro-N-{4-[({6-methyl-2-[(3S)-3-methylpiperazin-1-yl]quinazolin-4-yl}amino)methyl]phenyl}benzamide;
N-[4-({[2-(dimethylamino)-6,8-dimethylquinazolin-4-yl]amino}methyl)phenyl]-4-fluorobenzamide;
4-chloro-N-[4-({[2-(dimethylamino)-6-methylquinazolin-4-yl]amino}methyl)phenyl]benzamide
Ethyl 4-[4-({4-[(4-fluorobenzoyl)amino]benzyl}amino)-6-methylquinazolin-2-yl]piperazine-1-carboxylate;
4-fluoro-N-[4-({[6-methyl-2-(4-pyridin-2-ylpiperazin-1-yl)quinazolin-4-yl]amino}methyl)phenyl]benzamide;
N-(4-{[(2-azepan-1-yl-6-methylquinazolin-4-yl)amino]methyl}phenyl)-4-fluorobenzamide;
N-[4-({[2-(4-ethylpiperazin-1-yl)-6-methylquinazolin-4-yl]amino}methyl)phenyl]-4-fluorobenzamide; and
4-fluoro-N-{4-[({6-methyl-2-[methyl(pyridin-2-ylmethyl)amino]quinazolin-4-yl}amino)methyl]phenyl}benzamide.

16. The compound of claim 1, wherein the compound of formula I is selected from the group consisting of:
N-{4-[({2-(dimethylamino)-6-[6-(dimethylamino)pyridin-3-yl]quinazolin-4-yl}amino)methyl]phenyl}-4-fluorobenzamide;
N-[4-({[2-(dimethylamino)-6-fluoroquinazolin-4-yl]amino}methyl)phenyl]-4-fluorobenzamide;
N-[4-({[2-(dimethylamino)-7-isopropylquinazolin-4-yl]amino}methyl)phenyl]-4-fluorobenzamide;
6-chloro-N-[4-({[2-(dimethylamino)-7-isopropylquinazolin-4-yl]amino}methyl)phenyl]nicotinamide;
1-benzyl-N-[4-({[2-(dimethylamino)-7-isopropylquinazolin-4-yl]amino}methyl)phenyl]piperidine-4-carboxamide;
6-chloro-N-[4-({[2-(dimethylamino)-7-fluoro-8-methylquinazolin-4-yl]amino}methyl)phenyl]nicotinamide;
6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide;
N-[4-({[6-bromo-8-methyl-2-(methylamino)quinazolin-4-yl]amino}methyl)phenyl]-4-chlorobenzamide;
4-chloro-N-[4-({[6-(2-furyl)-8-methyl-2-(methylamino)quinazolin-4-yl]amino}methyl)phenyl]benzamide;
N-[4-({[2-(dimethylamino)-6-iodoquinazolin-4-yl]amino}methyl)phenyl]-4-fluorobenzamide;
N-{4-[({2-(dimethylamino)-6-[3-(dimethylamino)prop-1-yn-1-yl]quinazolin-4-yl}amino)methyl]phenyl}-4-fluorobenzamide;
Methyl 2-(dimethylamino)-4-({4-[(4-fluorobenzoyl)amino]benzyl}amino)quinazoline-6-carboxylate;
N-[4-({[2-(dimethylamino)-6-(hydroxymethyl)quinazolin-4-yl]amino}methyl)phenyl]-4-fluorobenzamide;
6-chloro-N-[4-({[2-(dimethylamino)-6-iodoquinazolin-4-yl]amino}methyl)phenyl]nicotinamide;
6-chloro-N-[4-({[2-(dimethylamino)-6-vinylquinazolin-4-yl]amino}methyl)phenyl]nicotinamide;
1-benzyl-N-[4-({[2-(dimethylamino)-6-iodoquinazolin-4-yl]amino}methyl)phenyl]piperidine-4-carboxamide;
1-benzyl-N-[4-({[2-(dimethylamino)-6-vinylquinazolin-4-yl]amino}methyl)phenyl]piperidine-4-carboxamide;
1-benzyl-N-[4-({[2-(dimethylamino)-8-methylquinazolin-4-yl]amino}methyl)phenyl]piperidine-4-carboxamide;
1-benzyl-N-(4-{[(6-methyl-2-pyrrolidin-1-ylquinazolin-4-yl)amino]methyl}phenyl)piperidine-4-carboxamide;
1-benzyl-N-{4-[({2-[(3R,5S)-3,5-dimethylpiperazin-1-yl]-6-methylquinazolin-4-yl}amino)methyl]phenyl}piperidine-4-carboxamide;
1-benzyl-N-[4-({[6-methyl-2-(4-pyridin-2-ylpiperazin-1-yl)quinazolin-4-yl]amino}methyl)phenyl]piperidine-4-carboxamide;
1-benzyl-N-[4-({[2-(2,5-dihydro-1H-pyrrol-1-yl)-6-methylquinazolin-4-yl]amino}methyl)phenyl]piperidine-4-carboxamide;
1-benzyl-N-{4-[({2-[(2-furylmethyl)amino]-6-methylquinazolin-4-yl}amino)methyl]phenyl}piperidine-4-carboxamide;
1-benzyl-N-[4-({[6-methyl-2-(4-pyrimidin-2-ylpiperazin-1-yl)quinazolin-4-yl]amino}methyl)phenyl]piperidine-4-carboxamide;
1-benzyl-N-[4-({[6-methyl-2-(4-pyrrolidin-1-ylpiperidin-1-yl)quinazolin-4-yl]amino}methyl)phenyl]piperidine-4-carboxamide;
N-(4-{[(2-azetidin-1-yl-6-methylquinazolin-4-yl)amino]methyl}phenyl)-1-benzylpiperidine-4-carboxamide;
1-benzyl-N-{4-[({6-methyl-2-[(3R)-3-methylpiperazin-1-yl]quinazolin-4-yl}amino)methyl]phenyl}piperidine-4-carboxamide;
N-[4-({[2-(dimethylamino)-7-iodoquinazolin-4-yl]amino}methyl)phenyl]-4-fluorobenzamide;
N-[4-({[2-(dimethylamino)-7-vinylquinazolin-4-yl]amino}methyl)phenyl]-4-fluorobenzamide;
N-[4-({[2-(dimethylamino)-7-ethylquinazolin-4-yl]amino}methyl)phenyl]-4-fluorobenzamide;
N-[4-({[7-cyano-2-(dimethylamino)quinazolin-4-yl]amino}methyl)phenyl]-4-fluorobenzamide;
N-[4-({[7-(aminomethyl)-2-(dimethylamino)quinazolin-4-yl]amino}methyl)phenyl]-4-fluorobenzamide;
N-{4-[({2-(dimethylamino)-7-[(dimethylamino)methyl]quinazolin-4-yl}amino)methyl]phenyl}-4-fluorobenzamide;
N-[4-({[2-(dimethylamino)-7-formylquinazolin-4-yl]amino}methyl)phenyl]-4-fluorobenzamide
N-[4-({[2-(dimethylamino)-7-(hydroxymethyl)quinazolin-4-yl]amino}methyl)phenyl]-4-fluorobenzamide;
N-[4-({[7-acetyl-2-(dimethylamino)quinazolin-4-yl]amino}methyl)phenyl]-4-fluorobenzamide
N-[4-({[2-(dimethylamino)-7-(1-hydroxyethyl)quinazolin-4-yl]amino}methyl)phenyl]-4-fluorobenzamide;
N-{4-[({2-(dimethylamino)-7-[(1E)-prop-1-en-1-yl]quinazolin-4-yl}amino)methyl]phenyl}-4-fluorobenzamide;
N-{4-[({2-(dimethylamino)-7-[(1Z)-prop-1-en-1-yl]quinazolin-4-yl}amino)methyl]phenyl}-4-fluorobenzamide;
N-[4-({[2-(dimethylamino)-7-(2-formylphenyl)quinazolin-4-yl]amino}methyl)phenyl]-4-fluorobenzamide;
N-[4-({[2-(dimethylamino)-7-(4-formylphenyl)quinazolin-4-yl]amino}methyl)phenyl]-4-fluorobenzamide;
N-[4-({[7-(2-chloropyridin-3-yl)-2-(dimethylamino)quinazolin-4-yl]amino}methyl)phenyl]-4-fluorobenzamide;
N-[4-({[7-(1-benzofuran-2-yl)-2-(dimethylamino)quinazolin-4-yl]amino}methyl)phenyl]-4-fluorobenzamide;
N-{4-[({2-(dimethylamino)-7-[(1E)-3,3-dimethylbut-1-en-1-yl]quinazolin-4-yl}amino)methyl]phenyl}-4-fluorobenzamide;
N-{4-[({2-(dimethylamino)-7-[(1E)-hex-1-en-1-yl]quinazolin-4-yl}amino)methyl]phenyl}-4-fluorobenzamide;
N-[4-({[7-cyclopropyl-2-(dimethylamino)quinazolin-4-yl]amino}methyl)phenyl]-4-fluorobenzamide;
6-chloro-N-[4-((1S)-1-{[2-(dimethylamino)-7-iodoquinazolin-4-yl]amino}ethyl)phenyl]nicotinamide;
6-chloro-N-[4-((1S)-1-{[2-(dimethylamino)-7-vinylquinazolin-4-yl]amino}ethyl)phenyl]nicotinamide;
6-chloro-N-{4-[(1S)-1-({2-(dimethylamino)-7-[(1E)-prop-1-en-1-yl]quinazolin-4-yl}amino)ethyl]phenyl}nicotinamide;
N-[4-({[2-(dimethylamino)-7-ethynylquinazolin-4-yl]amino}methyl)phenyl]-4-fluorobenzamide;
6-chloro-N-(4-{[(2-chloro-7-iodoquinazolin-4-yl)amino]methyl}phenyl)nicotinamide;
6-chloro-N-[4-({[2-(dimethylamino)-7-iodoquinazolin-4-yl]amino}methyl)phenyl]nicotinamide;
6-chloro-N-[4-({[2-(dimethylamino)-7-vinylquinazolin-4-yl]amino}methyl)phenyl]nicotinamide;
6-chloro-N-{4-[({2-(dimethylamino)-7-[(1E)-prop-1-en-1-yl]quinazolin-4-yl}amino)methyl]phenyl}nicotinamide;
N-[4-({[2-(dimethylamino)quinazolin-4-yl]amino}methyl)phenyl]-4-fluorobenzamide;
N-(4-{[(2-azetidin-1-ylquinazolin-4-yl)amino]methyl}phenyl)-4-fluorobenzamide;
N-[4-({[2-(cyclobutylamino)quinazolin-4-yl]amino}methyl)phenyl]-4-fluorobenzamide;
4-fluoro-N-[4-({[2-(4-methylpiperazin-1-yl)quinazolin-4-yl]amino}methyl)phenyl]benzamide;
4-fluoro-N-(4-{[(2-morpholin-4-ylquinazolin-4-yl)amino]methyl}phenyl)benzamide; and
N-[4-({[2-(ethylamino)quinazolin-4-yl]amino}methyl)phenyl]-4-fluorobenzamide.

17. The compound of claim 1, wherein the compound of formula I is selected from the group consisting of:
4-fluoro-N-(4-{[(2-pyrrolidin-1-ylquinazolin-4-yl)amino]methyl}phenyl)benzamide;
N-[4-({[2-(cyclopentylamino)quinazolin-4-yl]amino}methyl)phenyl]-4-fluorobenzamide;
N-[4-({[2-(cyclopropylamino)quinazolin-4-yl]amino}methyl)phenyl]-4-fluorobenzamide;
N-[4-({[2-(diethylamino)quinazolin-4-yl]amino}methyl)phenyl]-4-fluorobenzamide;
4-fluoro-N-(4-{[(2-piperidin-1-ylquinazolin-4-yl)amino]methyl}phenyl)benzamide;
4-fluoro-N-{4-[({2-[(2-furylmethyl)amino]quinazolin-4-yl}amino)methyl]phenyl}benzamide;
N-[4-({[2-(cyclohexylamino)quinazolin-4-yl]amino}methyl)phenyl]-4-fluorobenzamide;
tert-butyl N-[4-({4-[(4-fluorobenzoyl)amino]benzyl}amino)quinazolin-2-yl]glycinate;
N-[4-({4-[(4-fluorobenzoyl)amino]benzyl}amino)quinazolin-2-yl]glycine;
6-chloro-N-[4-({[2-(dimethylamino)quinazolin-4-yl]amino}methyl)phenyl]nicotinamide;
1-benzyl-N-[4-({[2-(dimethylamino)-7-methylquinazolin-4-yl]amino}methyl)phenyl]piperidine-4-carboxamide;
1-benzyl-N-[4-({[7-methyl-2-(methylamino)quinazolin-4-yl]amino}methyl)phenyl]piperidine-4-carboxamide;
N-(4-{[(2-azepan-1-yl-7-methylquinazolin-4-yl)amino]methyl}phenyl)-1-benzylpiperidine-4-carboxamide;
1-benzyl-N-[4-({[2-(ethylamino)-7-methylquinazolin-4-yl]amino}methyl)phenyl]piperidine-4-carboxamide;
1-benzyl-N-(4-{[(7-methyl-2-pyrrolidin-1-ylquinazolin-4-yl)amino]methyl}phenyl)piperidine-4-carboxamide;
N-(4-{[(2-azetidin-1-yl-7-methylquinazolin-4-yl)amino]methyl}phenyl)-1-benzylpiperidine-4-carboxamide;
1-benzyl-N-[4-({[7-methyl-2-(4-pyrrolidin-1-ylpiperidin-1-yl)quinazolin-4-yl]amino}methyl)phenyl]piperidine-4-carboxamide;
1-benzyl-N-[4-({[7-methyl-2-(4-pyrimidin-2-ylpiperazin-1-yl)quinazolin-4-yl]amino}methyl)phenyl]piperidine-4-carboxamide;
N-(4-{[(2-azetidin-1-yl-7-methylquinazolin-4-yl)amino]methyl}phenyl)-1-benzylpiperidine-4-carboxamide;
1-benzyl-N-{4-[({2-[3-(2-hydroxyethyl)piperazin-1-yl]-7-methylquinazolin-4-yl}amino)methyl]phenyl}piperidine-4-carboxamide;
1-benzyl-N-{4-[({2-[(2-hydroxyethyl)(methyl)amino]-7-methylquinazolin-4-yl}amino)methyl]phenyl}piperidine-4-carboxamide;
1-benzyl-N-{4-[({2-[[3-(dimethylamino)propyl](methyl)amino]-7-methylquinazolin-4-yl}amino)methyl]phenyl}piperidine-4-carboxamide;
1-benzyl-N-[4-({[7-methyl-2-(4-methylpiperazin-1-yl)quinazolin-4-yl]amino}methyl)phenyl]piperidine-4-carboxamide;
1-benzyl-N-{4-[({2-[benzyl(methyl)amino]-7-methylquinazolin-4-yl}amino)methyl]phenyl}piperidine-4-carboxamide;
1-benzyl-N-{4-[({7-methyl-2-[(3R)-3-methylpiperazin-1-yl]quinazolin-4-yl}amino)methyl]phenyl}piperidine-4-carboxamide;
1-benzyl-N-{4-[({2-[[2-(dimethylamino)ethyl](methyl)amino]-7-methylquinazolin-4-yl}amino)methyl]phenyl}piperidine-4-carboxamide;
1-benzyl-N-{4-[({7-methyl-2-[methyl(2-pyridin-2-ylethyl)amino]quinazolin-4-yl}amino)methyl]phenyl}piperidine-4-carboxamide;
1-benzyl-N-[4-({[2-(dimethylamino)quinazolin-4-yl]amino}methyl)phenyl]piperidine-4-carboxamide;
1-benzyl-N-[4-({[2-(methylamino)quinazolin-4-yl]amino}methyl)phenyl]piperidine-4-carboxamide;
1-benzyl-N-[4-({[2-(dimethylamino)-6-methylquinazolin-4-yl]amino}methyl)phenyl]piperidine-4-carboxamide;
1-benzyl-N-[4-({[6-methyl-2-(methylamino)quinazolin-4-yl]amino}methyl)phenyl]piperidine-4-carboxamide;
1-benzyl-N-[4-({[2-(dimethylamino)-7-vinylquinazolin-4-yl]amino}methyl)phenyl]piperidine-4-carboxamide;
1-benzyl-N-{4-[({2-(dimethylamino)-7-[(1E)-prop-1-en-1-yl]quinazolin-4-yl}amino)methyl]phenyl}piperidine-4-carboxamide;
1-benzyl-N-{4-[({2-(dimethylamino)-7-[(1E)-3,3-dimethylbut-1-en-1-yl]quinazolin-4-yl}amino)methyl]phenyl}piperidine-4-carboxamide;
1-benzyl-N-{4-[({2-(dimethylamino)-7-[(1Z)-prop-1-en-1-yl]quinazolin-4-yl}amino)methyl]phenyl}piperidine-4-carboxamide;
N-[4-({[2-(dimethylamino)-6-(trifluoromethyl)quinazolin-4-yl]amino}methyl)phenyl]-1-(4-fluorobenzyl)piperidine-4-carboxamide;
N-[4-({[2-azetidin-1-yl-6-(trifluoromethyl)quinazolin-4-yl]amino}methyl)phenyl]-1-(4-fluorobenzyl)piperidine-4-carboxamide;
1-(4-fluorobenzyl)-N-[4-({[2-pyrrolidin-1-yl-6-(trifluoromethyl)quinazolin-4-yl]amino}methyl)phenyl]piperidine-4-carboxamide;
1-(4-fluorobenzyl)-N-[4-({[2-(4-pyrimidin-2-ylpiperazin-1-yl)-6-(trifluoromethyl)quinazolin-4-yl]amino}methyl)phenyl]piperidine-4-carboxamide;
N-[4-({[2-(diethylamino)-6-(trifluoromethyl)quinazolin-4-yl]amino}methyl)phenyl]-1-(4-fluorobenzyl)piperidine-4-carboxamide;
N-[4-({[2-(cyclobutylamino)-6-(trifluoromethyl)quinazolin-4-yl]amino}methyl)phenyl]-1-(4-fluorobenzyl)piperidine-4-carboxamide;
1-(4-fluorobenzyl)-N-[4-({[2-(methylamino)-6-(trifluoromethyl)quinazolin-4-yl]amino}methyl)phenyl]piperidine-4-carboxamide;
4-chloro-N-[4-({[2-(dimethylamino)-6-(trifluoromethyl)quinazolin-4-yl]amino}methyl)phenyl]benzamide;
N-[4-({[2-azetidin-1-yl-6-(trifluoromethyl)quinazolin-4-yl]amino}methyl)phenyl]-4-chlorobenzamide;
4-chloro-N-[4-({[2-pyrrolidin-1-yl-6-(trifluoromethyl)quinazolin-4-yl]amino}methyl)phenyl]benzamide;
4-chloro-N-[4-({[2-(4-pyrimidin-2-ylpiperazin-1-yl)-6-(trifluoromethyl)quinazolin-4-yl]amino}methyl)phenyl]benzamide;
4-chloro-N-[4-({[2-(diethylamino)-6-(trifluoromethyl)quinazolin-4-yl]amino}methyl)phenyl]benzamide;
4-chloro-N-[4-({[2-(cyclobutylamino)-6-(trifluoromethyl)quinazolin-4-yl]amino}methyl)phenyl]benzamide;
4-chloro-N-[4-({[2-(methylamino)-6-(trifluoromethyl)quinazolin-4-yl]amino}methyl)phenyl]benzamide;
4-chloro-N-[4-({[2-[(2-furylmethyl)amino]-6-(trifluoromethyl)quinazolin-4-yl]amino}methyl)phenyl]benzamide;
N-(4-((2-dimethylamino)-6-(5-(dimethylamino)pyridine-2-yl)quinazolin-4-ylamino)methyl)phenyl)-4-fluorobenzamide;
4-((2-dimethylamino)-6-(3-(dimethylamino)phenyl)quinazolin-4-ylamino)methyl)N-4-fluorophenyl)benzamide;
Z-(4-((2-dimethylamino)-6-(styrylquinazolin-4-ylamino)-N-(4-fluorophenyl)benzamide;
4-((2-dimethylamino)-6-(3-vinylphenyl)quinazolin-4-ylamino)methyl)-N-(4-fluorophenyl)benzamide; and
E-(4-((2-dimethylamino)-6-(4-styrylphenyl)quinazolin-4-ylamino)methyl-N-(4-fluorophenyl)benzamide.

18. The compound of claim 1, wherein the compound of formula I is selected from the group consisting of:
E-4-((2-dimethylamino)-6-(prop-1-enyl)quinazolin-4-ylamino)methyl-N-(4-fluorophenyl)benzamide;
E-(4-((2-dimethylamino)-6-(hex-1-enyl)quinazolin-4-ylamino)methyl)-N-(4-fluorophenyl)benzamide;
(E)-N-{4-[({2-(dimethylamino)-6-(3,3-dimethylbut-1-enyl)quinazolin-4-ylamino)methyl)-N-(4-fluorophenyl)benzamide;
N-(4-chlorophenyl)-N′-[4-({[2-(dimethylamino)-7-methylquinazolin-4-yl]amino}methyl)phenyl]urea;
N-(4-chlorophenyl)-N′-[4-({[2-(dimethylamino)-6-methylquinazolin-4-yl]amino}methyl)phenyl]urea);
(N-(4-bromophenyl)-N′-[4-({[2-(dimethylamino)-7-methylquinazolin-4-yl]amino}methyl)phenyl]urea);
N-[4-({[2-(dimethylamino)-7-methylquinazolin-4-yl]amino}methyl)phenyl]-N′-(4-fluorophenyl)urea);
N-[4-({[2-(dimethylamino)-6-methylquinazolin-4-yl]amino}methyl)phenyl]-N′-(4-fluorophenyl)urea);
N-[4-({[2-(dimethylamino)quinazolin-4-yl]amino}methyl)phenyl]-N′-(4-fluorophenyl)urea;
6-chloro-N-[4-({[8-methyl-2-(methylamino)quinazolin-4-yl]amino}methyl)phenyl]nicotinamide and 6-(methylamino)-N-[4-({[8-methyl-2-(methylamino)quinazolin-4-yl]amino}methyl)phenyl]nicotinamide;
6-chloro-N-[4-({[2-(methylamino)-6-nitroquinazolin-4-yl]amino}methyl)phenyl]nicotinamide;
6-chloro-N-[4-({[2-(methylamino)-8-nitroquinazolin-4-yl]amino}methyl)phenyl]nicotinamide;
4-fluoro-N-[4-({[2-(methylamino)-6-nitroquinazolin-4-yl]amino}methyl)phenyl]benzamide;
N-[4-({[2-(dimethylamino)-6-nitroquinazolin-4-yl]amino}methyl)phenyl]-4-fluorobenzamide;
N-[4-({[6-nitro-2-(methylamino)quinazolin-4-yl]amino}methyl)phenyl]nicotinamide;
3,4-difluoro-N-[4-({[5-methyl-2-(methylamino)quinazolin-4-yl]amino}methyl)phenyl]benzamide;
4-fluoro-N-[4-({[8-methyl-2-(methylamino)quinazolin-4-yl]amino}methyl)phenyl]benzamide;
4-chloro-N-[4-({[8-methyl-2-(methylamino)quinazolin-4-yl]amino}methyl)phenyl]benzamide;
4-fluoro-N-[4-({[5-methyl-2-(methylamino)quinazolin-4-yl]amino}methyl)phenyl]benzamide;
4-chloro-N-[4-({[5-methyl-2-(methylamino)quinazolin-4-yl]amino}methyl)phenyl]benzamide;
6-chloro-N-[4-({[5-methyl-2-(methylamino)quinazolin-4-yl]amino}methyl)phenyl]nicotinamide;
4-chloro-N-[4-({[7-methyl-2-(methylamino)quinazolin-4-yl]amino}methyl)phenyl]benzamide;
4-chloro-N-[4-({[2-(dimethylamino)-7-methyl-quinazolin-4-yl]amino}methyl)phenyl]benzamide;
4-chloro-N-{4-[({7-methyl-2-[(2-pyridin-2-ylethyl)amino]quinazolin-4-yl}amino)methyl]phenyl}benzamide;
N-(4-{[(2-azepan-1-yl-7-methylquinazolin-4-yl)amino]methyl}phenyl)-4-chlorobenzamide;
N-[4-({[2-(dimethylamino)-7-methylquinazolin-4-yl]amino}methyl)phenyl]-4-fluorobenzamide;
4-fluoro-N-[4-({[7-methyl-2-(4-pyridin-2-ylpiperazin-1-yl)quinazolin-4-yl]amino}methyl)phenyl]benzamide;
N-{4-[({2-[[3-(dimethylamino)propyl](methyl)amino]-7-methylquinazolin-4-yl}amino)methyl]phenyl}-4-fluorobenzamide;
N-(4-{[(2-azetidin-1-yl-7-methylquinazolin-4-yl)amino]methyl}phenyl)-4-fluorobenzamide;
N-{4-[({2-[benzyl(methyl)amino]-7-methylquinazolin-4-yl}amino)methyl]phenyl}-4-fluorobenzamide;
4-fluoro-N-[4-({[7-methyl-2-(4-methylpiperazin-1-yl)quinazolin-4-yl]amino}methyl)phenyl]benzamide;
4-fluoro-N-{4-[({2-[(2S)-2-(methoxymethyl)pyrrolidin-1-yl]-7-methylquinazolin-4-yl}amino)methyl]phenyl}benzamide;
4-fluoro-N-(4-{[(7-methyl-2-piperidin-1-ylquinazolin-4-yl)amino]methyl}phenyl)benzamide;
4-fluoro-N-(4-{[(7-methyl-2-morpholin-4-ylquinazolin-4-yl)amino]methyl}phenyl)benzamide;
4-fluoro-N-(4-{[(7-methyl-2-piperazin-1-ylquinazolin-4-yl)amino]methyl}phenyl)benzamide;
4-fluoro-N-(4-{[(7-methyl-2-pyrrolidin-1-ylquinazolin-4-yl)amino]methyl}phenyl)benzamide;
N-{4-[({2-[ethyl(methyl)amino]-7-methylquinazolin-4-yl}amino)methyl]phenyl}-4-fluorobenzamide;
N-[4-({[2-(diethylamino)-7-methylquinazolin-4-yl]amino}methyl)phenyl]-4-fluorobenzamide;
4-fluoro-N-[4-({[7-methyl-2-(4-phenylpiperazin-1-yl)quinazolin-4-yl]amino}methyl)phenyl]benzamide;
4-fluoro-N-{4-[({7-methyl-2-[4-(2-oxo-2-pyrrolidin-1-ylethyl)piperazin-1-yl]quinazolin-4-yl}amino)methyl]phenyl}benzamide;
4-fluoro-N-{4-[({2-[(2-hydroxyethyl)(methyl)amino]-7-methylquinazolin-4-yl}amino)methyl]phenyl}benzamide;
N-{4-[({2-[4-(1,3-benzodioxol-5-ylmethyl)piperazin-1-yl]-7-methylquinazolin-4-yl}amino)methyl]phenyl}-4-fluorobenzamide;
4-fluoro-N-[4-({[7-methyl-2-(4-pyrimidin-2-ylpiperazin-1-yl)quinazolin-4-yl]amino}methyl)phenyl]benzamide;
4-fluoro-N-[4-({[2-(4-formylpiperazin-1-yl)-7-methylquinazolin-4-yl]amino}methyl)phenyl]benzamide;
Ethyl 4-[4-({4-[(4-fluorobenzoyl)amino]benzyl}amino)-7-methylquinazolin-2-yl]piperazine-1-carboxylate;
4-fluoro-N-(4-{[(2-{4-[2-(isopropylamino)-2-oxoethyl]piperazin-1-yl}-7-methylquinazolin-4-yl)amino]methyl}phenyl)benzamide;
4-fluoro-N-{4-[({2-[(2-methoxyethyl)(methyl)amino]-7-methylquinazolin-4-yl}amino)methyl]phenyl)}benzamide;
4-fluoro-N-{4-[({2-[(2-furylmethyl)(methyl)amino]-7-methylquinazolin-4-yl}amino)methyl]phenyl}benzamide;
4-fluoro-N-{4-[({7-methyl-2-[methyl(2-pyridin-2-ylethyl)amino]quinazolin-4-yl}amino)methyl]phenyl}benzamide;
N-[4-({[2-(4-acetyl-1,4-diazepan-1-yl)-7-methylquinazolin-4-yl]amino}methyl)phenyl]-4-fluorobenzamide;
4-fluoro-N-{4-[({2-[2-(2-hydroxyethyl)piperidin-1-yl]-7-methylquinazolin-4-yl}amino)methyl]phenyl}benzamide;
4-fluoro-N-{4-[({7-methyl-2-[(3R)-3-methylpiperazin-1-yl]quinazolin-4-yl}amino)methyl]phenyl}benzamide;
4-fluoro-N-[4-({[7-methyl-2-(4-pyrrolidin-1-ylpiperidin-1-yl)quinazolin-4-yl]amino}methyl)phenyl]benzamide;
N-[4-({[2-(4-ethylpiperazin-1-yl)-7-methylquinazolin-4-yl]amino}methyl)phenyl]-4-fluorobenzamide;
4-fluoro-N-{4-[({7-methyl-2-[(2S)-2-(pyrrolidin-1-ylmethyl)pyrrolidin-1-yl]quinazolin-4-yl}amino)methyl]phenyl}benzamide;
4-fluoro-N-(4-{[(7-methyl-2-{[3-(4-methylpiperazin-1-yl)propyl]amino}quinazolin-4-yl)amino]methyl}phenyl)benzamide;
4-fluoro-N-[4-({[7-methyl-2-(methylamino)quinazolin-4-yl]amino}methyl)phenyl]benzamide;
4-fluoro-N-[4-({[7-methyl-2-propylamino)quinazolin-4-yl]amino}methyl)phenyl]benzamide;
4-fluoro-N-{4-[({7-methyl-2-[(2-pyridin-2-ylethyl)amino]quinazolin-4-yl}amino)methyl]phenyl}benzamide;
N-{4-[({2-[(1-benzylpiperidin-4-yl)amino]-7-methylquinazolin-4-yl}amino)methyl]phenyl}-4-fluorobenzamide; and
4-fluoro-N-{4-[({7-methyl-2-[(3S)-3-methylpiperazin-1-yl]quinazolin-4-yl}amino)methyl]phenyl}benzamide.

19. The compound of claim 1, wherein the compound of formula I is selected from the group consisting of:
N-(4-{[(2-azepan-1-yl-7-methylquinazolin-4-yl)amino]methyl}phenyl)-4-fluorobenzamide;
N-[4-({[2-(3,3-dimethylpiperazin-1-yl)-7-methylquinazolin-4-yl]amino}methyl)phenyl]-4-fluorobenzamide;
4-fluoro-N-{4-[({7-methyl-2-[(2-pyrrolidin-1-ylethyl)amino]quinazolin-4-yl}amino)methyl]phenyl}benzamide;
4-bromo-N-[4-({[7-methyl-2-(methylamino)quinazolin-4-yl]amino}methyl)phenyl]benzamide;
4-bromo-N-[4-({[2-(dimethylamino)-7-methylquinazolin-4-yl]amino}methyl)phenyl]benzamide;
6-methyl-N-[4-({[7-methyl-2-(methylamino)quinazolin-4-yl]amino}methyl)phenyl]nicotinamide;
N-[4-({[2-(dimethylamino)-7-methylquinazolin-4-yl]amino}methyl)phenyl]-6-methylnicotinamide;
6-chloro-N-[4-({[2-(dimethylamino)-7-methylquinazolin-4-yl]amino}methyl)phenyl]nicotinamide;
N-[4-({[2-(dimethylamino)-7-methylquinazolin-4-yl]amino}methyl)phenyl]-1-methylpiperidine-4-carboxamide;
N-[4-({[2-(dimethylamino)-7-methylquinazolin-4-yl]amino}methyl)phenyl]-1-isobutylpiperidine-4-carboxamide;
1-cyclohexyl-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(2-furylmethyl)piperidine-4-carboxamide;
N-[4-({[2-(dimethylamino)-7-methylquinazolin-4-yl]amino}methyl)phenyl]-1-(4-methylbenzyl)piperidine-4-carboxamide;
N-[4-({[2-(dimethylamino)-7-methylquinazolin-4-yl]amino}methyl)phenyl]-1-(1H-imidazol-2-ylmethyl)piperidine-4-carboxamide;
1-butyl-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(4-methoxybenzyl)piperidine-4-carboxamide;
N-[4-({[2-(dimethylamino)-7-methylquinazolin-4-yl]amino}methyl)phenyl]-1-(4-fluorobenzyl)piperidine-4-carboxamide;
N-[4-({[2-(dimethylamino)-7-methylquinazolin-4-yl]amino}methyl)phenyl]-1-(2-fluorobenzyl)piperidine-4-carboxamide;
1-(4-chlorobenzyl)-N-[4-({[2-(dimethylamino)-7-methylquinazolin-4-yl]amino}methyl)phenyl]piperidine-4-carboxamide;
1-(2,4-difluorobenzyl)-N-[4-({[2-(dimethylamino)-7-methylquinazolin-4-yl]amino}methyl)phenyl]piperidine-4-carboxamide;
1-(3,4-difluorobenzyl)-N-[4-({[2-(dimethylamino)-7-methylquinazolin-4-yl]amino}methyl)phenyl]piperidine-4-carboxam-ide;
N-[4-({[2-(dimethylamino)-7-methylquinazolin-4-yl]amino}methyl)phenyl]-1-[4-(trifluoromethyl)benzyl]piperidine-4-carboxamide;
N-[4-({[2-(dimethylamino)-7-methylquinazolin-4-yl]amino}methyl)phenyl]-1-(pyridin-4-ylmethyl)piperidine-4-carboxamide;
1-(2-chloro-4-fluorobenzyl)-N-[4-({[2-(dimethylamino)-7-methylquinazolin-4-yl]amino}methyl)phenyl]piperidine-4-carboxamide;
1-[(6-chloropyridin-3-yl)methyl]-N-[4-({[2-(dimethylamino)-7-methylquinazolin-4-yl]amino}methyl)phenyl]piperidine-4-carboxamide;
N-[4-({[2-(dimethylamino)-7-methylquinazolin-4-yl]amino}methyl)phenyl]-1-(2,4,6-trifluorobenzyl)piperidine-4-carboxamide;
N-[4-({[2-(dimethylamino)-7-methylquinazolin-4-yl]amino}methyl)phenyl]-1-(3-fluorobenzyl)piperidine-4-carboxamide;
1-(2,5-difluorobenzyl)-N-[4-({[2-(dimethylamino)-7-methylquinazolin-4-yl]amino}methyl)phenyl]piperidine-4-carboxamide;
1-(4-chloro-3-fluorobenzyl)-N-[4-({[2-(dimethylamino)-7-methylquinazolin-4-yl]amino}methyl)phenyl]piperidine-4-carboxamide;
6-chloro-N-[4-({[7-methyl-2-(methylamino)quinazolin-4-yl]amino}methyl)phenyl]nicotinamide;
4-bromo-N-[4-({[8-methyl-2-(methylamino)quinazolin-4-yl]amino}methyl)phenyl]benzamide;
4-cyano-N-[4-({[8-methyl-2-(methylamino)quinazolin-4-yl]amino}methyl)phenyl]benzamide;
N-[4-((1S)-1-{[2-(dimethylamino)-7-methylquinazolin-4-yl]amino}ethyl)phenyl]piperidine-4-carboxamide;
1-(3,4-difluorobenzyl)-N-[4-((1S)-1-{[2-(dimethylamino)-7-methylquinazolin-4-yl]amino}ethyl)phenyl]piperidine-4-carboxamide;
(S)-6-chloro-N-(4-(1-(2-(dimethylamino)-7-methylquinazolin-4-ylamino)ethyl)phenyl)nicotinamide;
(R)-6-chloro-N-(4-(1-(2-(dimethylamino)-7-methylquinazolin-4-ylamino)ethyl)phenyl)nicotinamide;
(S)—N-(4-(1-(2-(dimethylamino)-7-methylquinazolin-4-ylamino)ethyl)phenyl)-4-fluorobenzamide;
(R)—N-(4-(1-(2-(dimethylamino)-7-methylquinazolin-4-ylamino)ethyl)phenyl)-4-fluorobenzamide;
(S)-6-chloro-N-(4-(1-(2-(dimethylamino)-8-methylquinazolin-4-ylamino)ethyl)phenyl)nicotinamide;
(S)-6-chloro-N-(4-(1-(2-(dimethylamino)-6-methylquinazolin-4-ylamino)ethyl)phenyl)nicotinamide;
(S)-4-fluoro-N-(4-(1-(8-methyl-2-(methylamino)quinazolin-4-ylamino)ethyl)phenyl)benzamide; and
(R)-4-fluoro-N-(4-(1-(8-methyl-2-(methylamino)quinazolin-4-ylamino)ethyl)phenyl)benzamide.

20. A pharmaceutical composition comprising at least one compound according to claim 1 and a pharmaceutically-acceptable carrier or diluent.

21. A pharmaceutical composition of claim 20, further comprising at least one other anti-cancer agent or cytotoxic agent.

22. The pharmaceutical composition of claim 21, wherein said other anti-cancer or cytotoxic agent is selected from the group consisting of 5-FU, leucovorin, irinotecan, bevacizumab, cetuximab, intraarterial floxuridine, oxaliplatin, gefitinib, and fluorouracil.

23. A method of inhibiting beta-catenin/Tcf-4 pathway comprising administering to a mammalian species in need thereof an effective amount of at least one compound according to claim 1.

24. A method for treating a condition or disorder comprising administering to a mammalian species in need thereof a therapeutically effective amount of at least one compound according to claim 1, wherein said condition or disorder is selected from the group consisting of proliferate diseases and cancers.

25. The method of claim 24, wherein said condition or disorder is colorectal cancer.

26. The method of claim 25, further comprising administering to a mammalian species in need thereof a therapeutically effective amount of at least one other anti-cancer or cytotoxic agent in combination with said at least one compound.

27. The method of claim 26, wherein said other anti-cancer or cytotoxic agent is selected from the group consisting of 5-FU, leucovorin, irinotecan, bevacizumab, cetuximab, intraarterial floxuridine, oxaliplatin, gefitinib, and fluorouracil.

28. A method of inhibiting the transcription of a gene selected from the group consisting of c-myc, cyclin D1, BMP4, KLF4, DHRS9/DHRL, MDR-1, Axin2, GPR49, ROR1, TIMP2, ID2, MSX1, and CSF2, comprising administering to a mammalian species in need thereof an effective amount of at least one compound according to claim 1.

29. A method for making a compound of formula I, 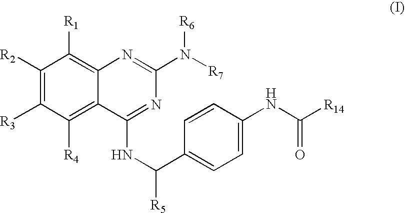
or an enantiomer, diastereomer, tautomer, or pharmaceutically acceptable salt or solvate thereof, comprising:
(a) reacting a compound of formula II, 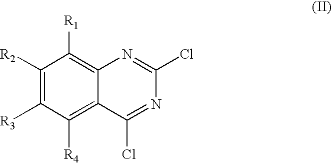
with an amine of formula III, 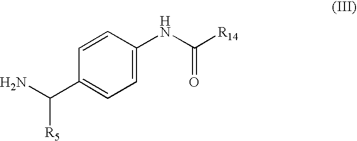
to provide a compound of formula IV; and 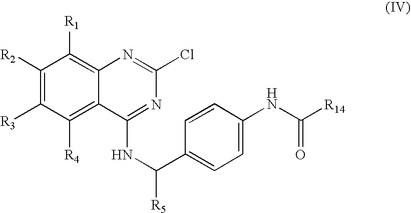
(b) further reacting the compound of formula IV with an amine of formula HNR₆R₇ to give the compound of formula I;
wherein the symbols of each of the above formulae have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
wherein: R₂ and R₃ together with the two contiguous carbon atoms to which R₂ and R₃ are bonded may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
each occurance of Rₐ is independently hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
each occurance of R_{b}, R_{c}, and R_{d} is independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
each occurance of Rₑ is independently alkyl or substituted alkyl alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

30. A method for making a compound of formula I, 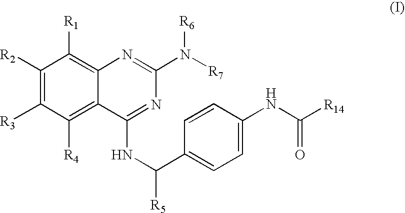
or an enantiomer, diastereomer, tautomer, or pharmaceutically acceptable salt or solvate thereof, comprising:
(a) reacting a compound of formula II, 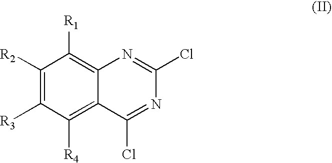
with an amine of formula V, 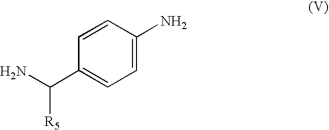
to provide a compound of formula VI; 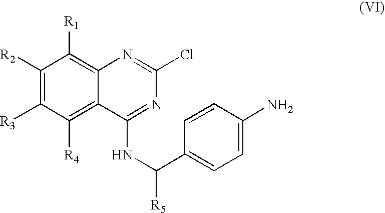
(b) reacting the compound of formula VI with an acid chloride of formula R₁₄(C═O)Cl, or an acid of formula of R₁₄C(═O)OH, to give a compound of formula IV; and 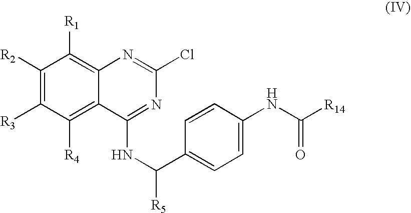
(c) further reacting the compound of formula IV with an amine of formula HNR₆R₇ to the compound of formula I;
wherein the symbols of each of the above formulae have the following meanings and are, for each occurrence, independently selected:
R₁, R₂, R₃, and R₄ are each independently hydrogen, halogen, cyano, nitro, CF₃, OCF₃, alkyl or substituted alkyl, alkenyl or substituted alkenyl, alkynyl or substituted alkynyl, cycloalkyl or substituted cycloalkyl, cycloalkenyl or substituted cycloalkenyl, heterocyclyl or substituted heterocyclyl, aryl or substituted aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ,
wherein: R₂ and R₃ together with the two contiguous carbon atoms to which R₂ and R₃ are bonded may optionally form a 5-7 membered optionally substituted carbocyclic ring or 5-7 membered optionally substituted heterocyclic ring;
R₅ is hydrogen, or alkyl or substituted alkyl;
R₆ and R₇ are each independently hydrogen, alkyl or substituted alkyl, cycloalkyl or substituted cycloalkyl, cycloalkenyl or substituted cycloalkenyl, heterocycle or substituted heterocycle, or aryl or substituted aryl, or said R₆ and R₇ together with the N to which they are bonded optionally form a heterocycle or substituted heterocycle;
R₁₄ is alkyl or substituted alkyl, NR_{b}R_{c}, cycloalkyl or substituted cycloalkyl, heterocycle or substituted heterocycle, or aryl or substituted aryl;
each occurance of independently Rₐ is hydrogen, alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl;
each occurance of R_{b}, R_{c} and R_{d} is independently independently hydrogen, alkyl or substituted alkyl, cycloalkyl or substituted cycloalkyl, heterocycle or substituted heterocycle, or aryl or substituted aryl, or said R_{b} and R_{c} together with the N to which they are bonded optionally form a heterocycle or substituted heterocycle; and
each occurance of Rₑ is independently alkyl or substituted alkyl, alkenyl or substituted alkenyl, alkynyl or substituted alkynyl, cycloalkyl or substituted cycloalkyl, cycloalkenyl or substituted cycloalkenyl, heterocycle or substituted heterocycle, or aryl or substituted aryl.

31. A compound of formula I prepared according to the method of claim 29.

32. A compound of formula I prepared according to the method of claim 30.
